(19) United States
(12) Patent Application Publication
(10) Pub. No.: US 2021/0046113 A1
(43) Pub. Date: Feb. 18, 2021

(54) DUAL-ACTIVATING COSTIMULATORY MOLECULE RECEPTOR AND USE THEREOF

(71) Applicants: Shanghai Cell Therapy Research Institute, Shanghai (CN); Shanghai Cell Therapy Group Co., Ltd., Shanghai (CN)

(72) Inventors: Qijun QIAN, Shanghai (CN); Huajun JIN, Shanghai (CN); Huimin XU, Shanghai (CN); Xiangzhen LIU, Shanghai (CN); Linfang LI, Shanghai (CN); Chao WANG, Shanghai (CN)

(73) Assignees: Shanghai Cell Therapy Research Institute, Shanghai (CN); Shanghai Cell Therapy Group Co., Ltd., Shanghai (CN)

(21) Appl. No.: 16/958,624

(22) PCT Filed: Dec. 26, 2018
(86) PCT No.: PCT/CN2018/123974

§ 371 Date: Oct. 14, 2020

(30) Foreign Application Priority Data
Dec. 28, 2017 (CN) 201711462081.7

(51) Int. Cl. A61K 35/17; C07K 14/705; A61P 35/00

(52) U.S. Cl. CPC A61K 35/17; C07K 14/70517; A61P 35/00; C07K 14/70521; C07K 14/70578

(57) ABSTRACT

Provided are a dual-activating costimulatory molecule receptor and the use thereof. The dual-activating costimulatory molecule receptor comprises the following elements from the N-terminus to the C-terminus: a selectable signal peptide, a costimulatory signal molecule-activating single-chain antibody, an extracellular hinge area, a transmembrane region and an intracellular costimulatory signal molecule. The dual-activating costimulatory molecule receptor can produce a strong clustering effect when co-modifying T-cells with first-generation CAR-T comprising a first signal, can kill tumor cells, and at the same time, does not trigger a strong T-cell immunity or cause potentially serious toxic side effects.

### CROSS REFERENCE TO RELATED APPLICATION

This application is a national stage application under 35 U.S.C. § 371 of International Application No. PCT/CN2018/123974, filed internationally on Dec. 26, 2018, which claims priority to Chinese Patent Application No. 201711462081.7 filed Dec. 28, 2017, the content of each of which is incorporated by reference in its entirety.

### INCORPORATION BY REFERENCE OF SEQUENCE LISTING

The present application is being filed along with a Sequence Listing in electronic format. The Sequence Listing is provided as a file entitled 699532000300SeqList.txt, created Oct. 14, 2020, which is 191,876 bytes in size. The information in the electronic format of the Sequence Listing is incorporated by reference in its entirety.

### TECHNICAL FIELD

The invention belongs to the field of cell biology and immunology, and relates to a dual-activating costimulatory molecule receptor and the use of treating malignant tumors by T cells modified with the receptor.

### BACKGROUND

Adoptive cell therapy (ACT) is to reinject the treated autologous or allogeneic immune cells (mainly autologous cells) to a tumor patient to directly kill tumor cells, or kill tumor cells by stimulating the body's immune response to achieve the therapy purpose. At present, adoptive cell therapy for tumors is developing rapidly, and good results has been achieved in the clinical treatment of various malignant tumors (Nature. 2016; Jun. 16; 534(7607):396-401); (Cell. 2016 Oct. 6; 167(2):405-418.e13). Tumor immune cell therapy is considered to be one of the most promising treatments for malignant tumors.

T cell activation requires the stimulation of two signals which are related to T cell activation. The TCR-CD3 complex on the surface of T cells binds to the antigen peptide-MHC molecule to provide the first signal for T cell activation and to determine the killing specificity of T cells; the costimulatory molecules (such as CD28) on the surface of T cells binds to the corresponding ligands (such as B7) to provide the second signal for T cell activation, and to promote T cell activation, proliferation and survival. However, the lack of or decreased expression of the first signal stimulus source (such as MHC molecules) and the second signal ligand (such as B7) of tumor cells cannot effectively provide signals related to T cell activation, and thus cannot activate T cell immune response. Extensive activation of T cell costimulatory molecules may bring strong toxic side effects.

The chimeric antigen receptor CAR (CAR) uses the extracellular single chain variable fragment (scFv) that specifically recognizes tumor antigens to activate the transmission of the intracellular signal CD3ζ or FccRIγ ITAM (immunoreceptor tyrosine-based activation motifs). However, the first-generation CAR receptor lacks the co-stimulatory signal of T cells, which causes T cells to exert only an instant effect, with a short duration in the body and little secretion of cytokines.

The second-generation and third-generation CARs combine the two signals required for T cell activation, and link the second signal CD28 or/and 4-1BB intracellular signal region directly to the CD3 molecule, thus avoiding the barrier that prevents T cells from being activated due to the lack of the second signal, such as B7, in tumor cells. After the first signal and the second signal are combined, the activation, proliferation and killing effect of T cells is greatly improved, which greatly increases the therapy effect. Based on the current understanding on the mechanism of T cell activation, CD28 and 4-1BB molecules can provide the second activation signal and further strengthen the TCR/CD3 signal.

However, no matter what kind of CAR-T cells, they can only provide stimulus signals to the modified T cells, but lack bystander effect and cannot activate surrounding T cells. Activation of surrounding T cells would result in a stronger clustering effect and cause a series of cascade reactions that activate T cell function.

### SUMMARY

Based on in-depth research and creative work, the inventor has designed a Dual Costimulatory Activated Receptor (DCR), which transmits the second signal related to T cell activation by an extracellular agonistic antibody for a costimulatory signal molecule. The modified T cells can not only activate their own costimulatory signals through the extracellular CD137 agonistic antibody, but also activate the intracellular costimulatory signals of the surrounding unmodified (un-activated) T cells upon contacting with the unmodified T cells, thereby promoting T cell activation, proliferation and survival. In particular, when DCR modifies T cells together with the first-generation CAR-T containing the first signal, a strong clustering effect that kills tumor cells can be produced. In addition, the effect of this dual activation is only limited to the T cells in contact with each other, and will not induce strong T cell immunity or cause potentially serious toxic side effects like a CD137 agonistic antibody.

The followings are provided in the present disclosure:

One aspect of the present disclosure relates to an isolated polypeptide, comprising, from N-terminus to C-terminus, the following elements:

an optional signal peptide, a polypeptide that activates a costimulatory signal molecule (such as an agonistic single-chain antibody for the costimulatory signal molecule or a ligand of the costimulatory signal molecule), an extracellular hinge region, a transmembrane region, and a intracellular costimulatory signal molecule.

In one or more embodiments of the present disclosure, the polypeptide is characterized by any 1, 2, 3, 4 or 5 of the following items (1) to (5):

(1) the signal peptide is a membrane protein signal peptide; preferably, the signal peptide is one or more peptides selected from the group consisting of a CD8 signal peptide, a CD28 signal peptide, and a CD4 signal peptide; preferably, the signal peptide is a CD8 signal peptide; preferably, the amino acid sequence of the CD8 signal peptide is as shown in SEQ ID NO: 1;

(2) the agonistic single-chain antibody for the costimulatory signal molecule is any one or more of members selected from the group consisting of a CD137 agonistic single-chain antibody, a CD28 agonistic single-chain antibody and a CD40 agonistic single-chain antibody; the ligand of the costimulatory signal molecule is any one or more of members selected from the group consisting of a ligand of CD137, a ligand of CD28 and a ligand of CD40;

preferably, the amino acid sequence of the CD137 agonistic single-chain antibody is as shown in SEQ ID NO: 2;

preferably, the amino acid sequence of the CD28 agonistic single-chain antibody is as shown in SEQ ID NO: 31;

preferably, the amino acid sequence of the CD40 agonistic single-chain antibody is as shown in SEQ ID NO: 55;

preferably, the ligand of CD137 is 4-1BBL;

preferably, the ligand of CD28 is CD80/CD86;

preferably, the ligand of CD40 is CD40L;

(3) the extracellular hinge region is any one or more of members selected from the group consisting of a IgG4Fc CH2CH3 hinge region, a CD28 hinge region and a CD8 hinge region;

preferably, the extracellular hinge region is a CD8 hinge region;

preferably, the amino acid sequence of the CD8 hinge region is as shown in SEQ ID NO: 3;

preferably, the extracellular hinge region is an IgG4Fc CH2CH3 hinge region;

preferably, the amino acid sequence of the IgG4Fc CH2CH3 hinge region is as shown in SEQ ID NO: 56;

(4) the transmembrane region is any one or more of members selected from the group consisting of a CD28 transmembrane region, a CD8 transmembrane region, a CD3ζ transmembrane region, a CD134 transmembrane region, a CD137 transmembrane region, an ICOS transmembrane region and a DAP10 transmembrane region; preferably, the transmembrane region is a CD28 transmembrane region; preferably, the amino acid sequence of the CD28 transmembrane region is as shown in SEQ ID NO: 4;

(5) the intracellular costimulatory signal molecule is any one or more of members selected from the group consisting of a CD28 intracellular domain, a CD134/OX40 intracellular domain, a CD137/4-1BB intracellular domain, a LCK intracellular domain, an ICOS intracellular domain and a DAP10 intracellular domains; preferably, the intracellular costimulatory signal molecule is a CD28 intracellular domain and/or a CD137 intracellular domain; preferably, the amino acid sequence of the CD28 intracellular domain is as shown in SEQ ID NO: 5; preferably, the amino acid sequence of the CD137 intracellular domain is as shown in SEQ ID NO: 6.

In one or more embodiments of the present disclosure, the polypeptide comprises, from N-terminus to C-terminus, the following elements:

an optional CD8 signal peptide, a CD137 agonistic single-chain antibody, a CD8 extracellular hinge region, a CD28 transmembrane region, a CD28 intracellular domain and/or a CD137 intracellular domain;

an optional CD8 signal peptide, a CD28 agonistic single-chain antibody, a CD8 extracellular hinge region, a CD28 transmembrane region, a CD28 intracellular domain and/or a CD137 intracellular domain; or

an optional CD8 signal peptide, a CD40 agonistic single-chain antibody, an IgG4Fc CH2CH3 hinge region, a CD28 transmembrane region, a CD28 intracellular domain and/or a CD137 intracellular domain;

In one or more embodiments of the present disclosure, the polypeptide is as shown in FIG. 1A-1 to FIG. 1D-1.

In one or more embodiments of the present disclosure, the polypeptide is as shown in FIG. 1A-2 to FIG. 1D-2.

In one or more embodiments of the present disclosure, the polypeptide is as shown in FIG. 1A-3 to FIG. 1D-3.

In one or more embodiments of the present disclosure, the amino acid sequence of the polypeptide is as shown in any one of SEQ ID NOs: 7 to 14;

any one of SEQ ID NOs: 32 to 39; or

any one of SEQ ID NOs: 57 to 64.

Another aspect of the present disclosure relates to an isolated polynucleotide encoding the isolated polypeptide according to any embodiment of the present disclosure; preferably, the sequence of the isolated polynucleotide is as shown in any of SEQ ID NO: 15 or 22;

any one of SEQ ID NOs: 40 to 47; or

any one of SEQ ID NOs: 65 to 72.

Another aspect of the present disclosure relates to a nucleic acid construct comprising the polynucleotide of the present disclosure.

Another aspect of the present disclosure relates to a recombinant vector, comprising the polynucleotide of the present disclosure or the nucleic acid construct of the present disclosure; preferably, the recombinant vector is a recombinant cloning vector, a recombinant eukaryotic expression plasmid or a recombinant viral vector; preferably, the recombinant expression vector is a recombinant transposon vector; preferably, the transposon vector contains a transposition element selected from the group consisting of piggybac, sleeping beauty, frogprince, Tn5, and Ty; preferably, the recombinant expression vector is a recombinant vector obtained by recombining the polynucleotide of the present disclosure and a PS328b vector.

Another aspect of the present disclosure relates to a recombinant vector combination, comprising a first recombinant vector and a second recombinant vector, wherein:

The first recombinant vector is the recombinant vector of the present disclosure,

The second recombinant vector contains a coding sequence of a first-generation chimeric antigen receptor; preferably, the first-generation chimeric antigen receptor is one that targets mesothelin, Muc1 or EGFR; preferably, the amino acid sequence of the first-generation chimeric antigen receptor is as shown in SEQ ID NO: 23, SEQ ID NO: 48 or SEQ ID NO: 73; preferably, the nucleic acid sequence of the first-generation chimeric antigen receptor is as shown in SEQ ID NO: 24, SEQ ID NO: 49 or SEQ ID NO: 74;

preferably, the second recombinant vector is a recombinant PNB328 vector.

wherein, the “first” and “second” in the above-mentioned “first recombinant vector” and “second recombinant vector” are only for the purpose of distinguishing, and do not mean the order.

Another aspect of the present disclosure relates to a recombinant host cell, wherein the cell contains the polynucleotide of the present disclosure, the nucleic acid construct of the present disclosure, the recombinant vector of the present disclosure, or the combination of the recombinant vectors of the present disclosure; preferably, the recombinant host cell is a recombinant mammalian cell; preferably, the recombinant host cell is a recombinant T cell; preferably, the recombinant T cell is a recombinant peripheral blood mononuclear cell.

Yet another aspect of the present disclosure relates to a T cell expressing the polypeptide according to any claim of the present disclosure and a first-generation chimeric antigen receptor; preferably, the recombinant T cell is a recombinant peripheral blood mononuclear cell; preferably, the first-generation chimeric antigen receptor is one that targets mesothelin, Muc1 or EGFR; preferably, the amino acid sequence of the first-generation chimeric antigen receptor is as shown in SEQ ID NO: 23, SEQ ID NO: 48 or SEQ ID NO: 73.

Another aspect of the present disclosure relates to a pharmaceutical composition comprising any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure; optionally, further comprising a pharmaceutically acceptable excipient.

Another aspect of the present disclosure relates to use of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure in the preparation of medicament for treating and/or preventing a cancer; preferably, the cancer is one that abnormally expresses mesothelin, Muc1 or EGFR on the surface of its cancer cells; preferably, the cancer is selected from the group consisting of adenocarcinoma, lung cancer, colon cancer, colorectal cancer, breast cancer, ovarian cancer, cervical cancer, gastric cancer, bile duct cancer, gallbladder cancer, esophageal cancer, pancreatic cancer or prostate cancer.

Another aspect of the present disclosure relates to use of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure in the preparation of medicament for inhibiting a cancer cell; preferably, the cancer cell is one that abnormally expresses mesothelin, Muc1 or EGFR on the cell surface; preferably, the cancer cell is selected from the group consisting of: cancer cells of adenocarcinoma, lung cancer, colon cancer, colorectal cancer, breast cancer, ovarian cancer, cervical cancer, gastric cancer, bile duct cancer, gallbladder cancer, esophageal cancer, pancreatic cancer or prostate cancer.

Another aspect of the present disclosure relates to a method of inhibiting a cancer cell in vivo or in vitro, comprising the step of administering to the cancer cell an effective amount of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure; preferably, the cancer cell is one that abnormally expresses mesothelin, Muc1 or EGFR on the cell surface; preferably, the cancer cell is selected from the group consisting of: cancer cells of adenocarcinoma, lung cancer, colon cancer, colorectal cancer, breast cancer, ovarian cancer, cervical cancer, gastric cancer, bile duct cancer, gallbladder cancer, esophageal cancer, pancreatic cancer or prostate cancer.

Another aspect of the present disclosure relates to a method of treating and/or preventing cancer, comprising the step of administering to a subject in need thereof an effective amount of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure; preferably, the cancer is one that abnormally expresses mesothelin, Muc1 or EGFR on the surface of its cancer cells; preferably, the cancer is selected from the group consisting of adenocarcinoma, lung cancer, colon cancer, colorectal cancer, breast cancer, ovarian cancer, cervical cancer, gastric cancer, bile duct cancer, gallbladder cancer, esophageal cancer, pancreatic cancer or prostate cancer.

Another aspect of the present disclosure relates to use of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure in the preparation of a medicament for promoting the secretion of a cytokine, wherein the cytokine is any one or more of members selected from the group consisting of IL-2, IL-4, IL-6, IL-10, TNF-α and IFN-γ.

Another aspect of the present disclosure relates to a method of promoting the secretion of a cytokine by T cells in vivo or in vitro, comprising the step of administering to the T cells an effective amount of any polypeptide of the present disclosure, the polynucleotide of the present disclosure, the nucleic acid construct of the present disclosure, the recombinant vector of the present disclosure, the combination of the recombinant vectors of the present disclosure, the recombinant host cell of the present disclosure or the T cell of the present disclosure, wherein the cytokine is any one or more of members selected from the group consisting of IL-2, IL-4, IL-6, IL-10, TNF-α and IFN-γ.

In the present disclosure, unless otherwise specified, the scientific and technical terms used herein have the meanings commonly understood by those skilled in the art. Moreover, the laboratory procedures of cell culture, molecular genetics, nucleic acid chemistry, and immunology used herein are all routine procedures widely used in the corresponding fields. Meanwhile, in order to better understand the present disclosure, definitions and explanations of related terms are provided below.

In the present disclosure, the term “isolated” or “be isolated” refers to substances obtained from the natural state by artificial means. If a certain “isolated” substance or component appears in nature, its natural environment may have changed, or the substance has been separated from the natural environment, or both. For example, if a certain non-isolated polynucleotide or polypeptide naturally exists in a living animal, the same polynucleotide or polypeptide with high purity isolated from its natural state is called “isolated”. The term “isolated” or “be isolated” does not exclude the inclusion of artificial or synthetic substances, nor does it exclude the presence of other impure materials that do not affect the activity of the substance.

In the present disclosure, the term “vector” refers to a nucleic acid delivery vehicle into which polynucleotides can be inserted. When the vector can express the protein encoded by the inserted polynucleotide, the vector is called an expression vector. The vector can be introduced into a host cell by transformation, transduction or transfection, so that the genetic material elements carried by the vector can be expressed in the host cell. Vectors are well known to those skilled in the art, including but not limited to: plasmids; phagemids; cosmids; artificial chromosomes, such as yeast artificial chromosomes (YAC), bacterial artificial chromosomes (BAC) or P1 derived artificial chromosomes (PAC); phages such as lambda phage or M13 phage and animal viruses, etc. Animal viruses that can be used as vectors include, but are not limited to, retroviruses (including lentiviruses), adenoviruses, adeno-associated viruses, herpes viruses (such as herpes simplex virus), poxviruses, baculoviruses, papillomaviruses, and papovaviruses, (e.g. SV40). A vector can contain a variety of elements that control expression, including but not limited to promoter sequences, transcription initiation sequences, enhancer sequences, selection elements and reporter genes. In addition, the vector may also contain an origin of replication.

In the present disclosure, the term “host cell” refers to a cell that can be used to introduce a vector, which includes, but is not limited to, prokaryotic cells such as Escherichia coli or B. subtilis, fungal cells such as yeast cells or Aspergillus, insect cells such as S2 Drosophila melanogaster cells or Sf9, or animal cells such as fibroblasts, CHO cells, COS cells, NSO cells, HeLa cells, BHK cells, HEK 293 cells or human cells.

In the present disclosure, the term “chimeric antigen receptor” (CAR) is an artificially modified receptor which can anchor the specific molecules (such as antibodies) recognizing tumor antigens to immune cells (such as T cells), so that the immune cells can recognize tumor antigens or virus antigens and kill tumor cells or virus-infected cells.

In the present disclosure, the term “CD137”, the NCBI genebank Access ID of 3604, is expressed in T cells and can promote the proliferation and activation of T cells. In the second-generation chimeric antigen receptor-modified T cell therapy, it is often used as an intracellular costimulatory signal to enhance the activation and proliferation of T cells.

In the present disclosure, the term “CD28”, the NCBI genebank Access ID of 940, is expressed in T cells and can promote the proliferation and activation of T cells. In the second-generation chimeric antigen receptor-modified T cell therapy, it is often used as an intracellular costimulatory signal to enhance the activation and proliferation of T cells.

In the present disclosure, the term “CD40”, the NCBI genebank Access ID of 958, is expressed in T cells and can promote the proliferation and activation of T cells. In the second-generation chimeric antigen receptor-modified T cell therapy, it is often used as an intracellular costimulatory signal to enhance the activation and proliferation of T cells.

In the present disclosure, the term “single-chain antibody” or “single-chain antibody variable fragment (scFv)” refers to an antibody fragment formed by linking the amino acid sequences of V_{L} region and V_{H} region of an antibody via a Linker, which has the ability to bind to an antigen. Wherein the VL and VH domains pair to form a monovalent molecule by a connector that can produce a single polypeptide chain (see, for example, Bird et al., Science 242:423-426 (1988) and Huston et al., Proc. Natl. Acad. Sci. USA 85:5879-5883 (1988)) Such scFv molecules may have the general structure: NH₂-V_{L}-linker-V_{H}—COOH or NH₂-V_{H}-linker-V_{L}-COOH. A suitable linker in the art consists of repeated GGGGS amino acid sequence(s) or variants thereof. For example, a linker having an amino acid sequence of (GGGGS)₄ can be used, but variants thereof can also be used (Holliger et al. Proc. Natl. Acad. Sci. USA 90: 6444-6448). Other linkers that can be used in the present disclosure are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol.

In the present disclosure, the term “signal(s) related to T cell activation” refers to the two signals required for T cell activation, that is, the TCR-CD3 complex on the surface of T cells binding to the antigen peptide-MHC molecule to provide the first signal for T cell activation and determine the killing specificity of T cells; the costimulatory molecules (such as CD28) on the surface of T cells binding to the corresponding ligands (such as B7) to provide the second signal for T cell activation, promoting T cell activation, proliferation and survival.

In the present disclosure, the immunoreceptor tyrosine activation motif is a CD3ζ and/or FccRIγ tyrosine activation motif; preferably, the immunoreceptor tyrosine activation motif is a CD3ζ tyrosine activation motif, amino acid sequence of which is as shown in SEQ ID NO: 25.

The term “co-stimulating molecule” or “costimulatory signal molecule” in the present disclosure refers to some adhesion molecules on the surface of immune cells, such as CD28, CD134/OX40, CD137/4-1BB, CD40, etc., which activate the second signal of the immune cells, enhance the proliferation and the cytokines secretion of the immune cells, and improve the survival of activated immune cells by binding to their ligands.

In the present disclosure, the term “PB” transposon is short for Piggybac. Transposon is a mobile genetic factor. A stretch of DNA sequence can be copied or cut separately from the original position, inserted into another site after circularization, which regulates the downstream genes. This process is called transposition. Due to the function of the transposon on the vector, meso G1 CAR or 137 DCR is integrated into the T cell genome.

Antibodies are divided into agonistic and inhibitory antibodies. In the present disclosure, the term “extracellular agonistic antibody” refers to a antibody anchored on the surface of the cell membrane and bound to a acting site of a cell surface molecule (i.e., the position where ligand and receptor bind to each other) to promote cell biological functions. CD137 extracellular agonistic antibody is considered to be a unique surface molecule of T cells, as CD137 molecule exists on the surface of most T cells. CD137 extracellular agonistic antibody can effectively recognize and activate the CD137 molecular signal and generate the second signal. CD137 can replace the second signal effect of APC.

In the present disclosure, the term “bystander effect” means that, for tumor cells or virus-infected cells, a single CAR-T cell can only activate its own second signal, but cannot further activate surrounding T cells, so that the surrounding T cells cannot provide a series of activated T cell functions.

In the present disclosure, the term “clustering effect” means that a single modified T cell can continuously recruit and activate surrounding un-activated T cells, and activate downstream signaling pathways of the surrounding T cells, leading to multiple T cells activation, proliferation and other functions.

In the present disclosure, the term “mesothelin” is also known as MSLN, meso, mesothelin, with the NCBI genebank Access ID of 10232. Initially, the synthesized mesothelin is a 69 kDa cell surface protein. During the maturation, the synthesized mesothelin is lysed to two parts by furin, wherein, the 40-kDa fragment at the C-terminal is anchored on the membrane, and the 32-kDa fragment at the N-terminus is released in a dissolved form, called pegakaryocyte potentiation factor (MPF). The so-called mesothelin refers to the fragment anchored on the membrane. Mesothelin is overexpressed in various malignant tumors such as pancreatic cancer, mesothelioma, ovarian cancer and lung adenocarcinoma, and it is a promising target for cell therapy. The full-length mesothelin protein can be divided into three segments, Region I (296-390), II (391-486) and III (487-598).

In the present disclosure, the term “Muc1” is also known as mucin, with the NCBI genebank Access ID of 4582. Muc1 is a type I transmembrane glycoprotein with a high molecular weight (>200 kD), mostly linked to Ser/Thr on the polypeptide backbone by 0-glycosidic bonds. Under normal circumstances, it is mainly expressed in a variety of tissues and organs near the lumen surface or glandular lumen surface of epithelial cells, with apical expression and polar distribution. When tumors occur, Muc1 protein can be abnormally expressed on the surface of tumor cells, and its expression level can reach more than 100 times of normal. Moreover, its polar distribution on the cell surface is lost, and it can be evenly distributed on the entire cell surface. In addition, due to incomplete glycosylation, the structure of Muc1 protein has also changed, with new sugar chains and peptide epitopes appeared.

In the present disclosure, the term “EGFR” is also known as epidermal growth factor receptor, ErbB-1 or HER1, with the NCBI genebank Access ID of 1956. EGFR is widely distributed on the cell surface of mammalian epithelial cells, fibroblasts, glial cells, keratinocytes, etc. EGFR signaling pathway plays an important role in physiological processes such as cell growth, proliferation and differentiation. EGFR is related to the inhibition of tumor cell proliferation, angiogenesis, tumor invasion, metastasis and apoptosis. The overexpression of EGFR plays an important role in the evolution of malignant tumors, such as glial cell carcinoma, kidney cancer, lung cancer, prostate cancer, pancreatic cancer, breast cancer and so on.

In the present disclosure, the term “a pharmaceutically acceptable carrier/excipient” refers to a carrier and/or an excipient that are pharmacologically and/or physiologically compatible with a subject and active ingredient(s), which is well known in the art (see, for example, Remington's Pharmaceutical Sciences, Gennaro A R Ed., 19th edition, Pennsylvania: Mack Publishing Company, 1995), including but not limited to, pH adjusting agent, surfactant, adjuvant, ion strength enhancer. For example, the pH adjusting agent includes, but is not limited to, phosphate buffer; the surfactant includes, but is not limited to, cationic, anionic or non-ionic surfactant, such as Tween-80; the ion strength enhancer includes, but is not limited to, sodium chloride.

In the present disclosure, the term “effective amount” refers to an amount sufficient to obtain or at least partially obtain the desired effect. For example, an effective amount for preventing a disease (such as a tumor) refers to an amount sufficient to prevent, prohibit, or delay the occurrence of a disease (such as a tumor); an effective amount for treating a disease refers to an amount sufficient to cure or at least partially prevent a disease and its complications in a patient having the disease. It is completely within the abilities of those skilled in the art to determine such an effective amount. For example, the effective amount for therapy will depend on the severity of the disease to be treated, the overall state of the patient's own immune system, the patient's general conditions such as age, weight and sex, the administration route of the drug, and other treatments administered simultaneously and so on.

In the present disclosure, the subject may be a mammal, such as a human.

The Beneficial Effects of the Disclosure

When DCR modifies T cells together with the first-generation CAR-T containing the first signal, a strong clustering effect can be produced, which kills tumor cells. Meanwhile, the effect of this dual activation is only limited to the T cells in contact with each other, and will not induce strong T cell immunity or cause potentially serious toxic side effects like that from injecting a CD137 agonistic antibody. The T cells modified with CD137 dual costimulatory activated receptor combined with chimeric antigen receptor against mesothelin can specifically kill tumor cell lines with high mesothelin expression and is superior to the first-generation and second-generation CAR-T against mesothelin, with little or no killing effect on non-expressing tumor cell lines, having high efficiency and high specificity. While maintaining the efficacy of the first and second-generation CARs, the T cells activated by the CD137 dual costimulatory molecule activated receptor can activate their own second signal; the stronger the tumor-specific antigen, the stronger the first signal CD3ζ activation, and the stronger the second signal related to T cell activation transmitted by the CD137 extracellular agonistic antibody; gathering around the tumor, and continuously recruiting and activating the surrounding un-activated T cells, and activating the downstream signal pathways of T cells, causing T cell cascade activation, proliferation and survival.

When DCR modifies T cells together with the first-generation CAR-T containing the first signal, a strong clustering effect can be produced which kills tumor cells. Meanwhile, the effect of this dual activation is only limited to the T cells in contact with each other, and will not induce strong T cell immunity or cause potentially serious toxic side effects like that from injecting a CD28 agonistic antibody. The T cells modified with CD28 dual costimulatory activated receptor combined with chimeric antigen receptor against Muc1 can specifically kill tumor cell lines with high Muc1 expression and is superior to the first-generation and second-generation CAR-T against Muc1, with little or no killing effect on non-expressing tumor cell lines, having high efficiency and high specificity. While maintaining the efficacy of the first and second-generation CARs, the T cells activated by the CD28 dual costimulatory activated receptor can activate their own second signal; the stronger the tumor-specific antigen, the stronger the first signal CD3 activation, and the stronger the second signal related to T cell activation transmitted by the CD28 extracellular agonistic antibody; gathering around the tumor, and continuously recruiting and activating the surrounding un-activated T cells, and activating the downstream signal pathways of T cells, causing T cell cascade activation, proliferation and survival.

When DCR modifies T cells together with the first-generation CAR-T containing the first signal, a strong clustering effect can be produced, which kills tumor cells. Meanwhile, the effect of this dual activation is only limited to the T cells in contact with each other, and will not induce strong T cell immunity or cause potentially serious toxic side effects like that from injecting a CD40 agonistic antibody. The T cells modified with CD40 dual costimulatory activated receptor combined with chimeric antigen receptor against EGFR can specifically kill tumor cell lines with high EGFR expression and is superior to the first-generation and second-generation CAR-T against EGFR, with little or no killing effect on non-expressing tumor cell lines, having high efficiency and high specificity. While maintaining the efficacy of the first and second-generation CARs, the T cells activated by the CD40 dual costimulatory molecule activated receptor can activate their own second signal; the stronger the tumor-specific antigen, the stronger the first signal CD3ζ activation, and the stronger the second signal related to T cell activation transmitted by the CD40 extracellular agonistic antibody; gathering around the tumor, and continuously recruiting and activating the surrounding un-activated T cells, and activating the downstream signal pathways of T cells, causing T cell cascade activation, proliferation and survival.

### BRIEF DESCRIPTION OF DRAWINGS

FIG. 1A-1: Schematic diagram of the structure of CD137 dual-activating costimulatory molecule receptor 137DCR1.

FIG. 1B-1: Schematic diagram of the structure of CD137 dual-activating costimulatory molecule receptor 137DCR2.

FIG. 1C-1: Schematic diagram of the structure of CD137 dual-activating costimulatory molecule receptor 137DCR3.

FIG. 1D-1: Schematic diagram of the structure of CD137 dual-activating costimulatory molecule receptor 137DCR4.

FIG. 1E-1: Schematic diagram of the structure of meso G1 CAR.

FIG. 1F-1: Schematic diagram of the structure of meso G2 CAR.

FIG. 1A-2: Schematic diagram of the structure of CD28 dual-activating costimulatory molecule receptor 28DCR1.

FIG. 1B-2: Schematic diagram of the structure of CD28 dual-activating costimulatory molecule receptor 28DCR2.

FIG. 1C-2: Schematic diagram of the structure of CD28 dual-activating costimulatory molecule receptor 28DCR3.

FIG. 1D-2: Schematic diagram of the structure of CD28 dual-activating costimulatory molecule receptor 28DCR4.

FIG. 1E-2: Schematic diagram of the structure of Muc1 G1 CAR.

FIG. 1F-2: Schematic diagram of the structure of Muc1 G2 CAR.

FIG. 1A-3: Schematic diagram of the structure of CD40 dual-activating costimulatory molecule receptor 40DCR1.

FIG. 1B-3: Schematic diagram of the structure of CD40 dual-activating costimulatory molecule receptor 40DCR2.

FIG. 1C-3: Schematic diagram of the structure of CD40 dual-activating costimulatory molecule receptor 40DCR3.

FIG. 1D-3: Schematic diagram of the structure of CD40 dual-activating costimulatory molecule receptor 40DCR4.

FIG. 1E-3: Schematic diagram of the structure of EGFR G1 CAR.

FIG. 1F-3: Schematic diagram of the structure of EGFR G2 CAR.

FIG. 2A-1: CD3ζ expression profile in the dual-activating chimeric antigen receptor mesothelin CAR-T cells. The internal control is GADPH.

FIG. 2B-1: copy numbers of 137DCR1, 137DCR2, 137DCR3 in the dual-activating chimeric antigen receptor mesothelin CAR-T cells.

FIG. 2A-2: CD3ζ expression profile in the dual-activating chimeric antigen receptor Muc1 CAR-T cells. The internal control is GADPH.

FIG. 2B-2: Copy numbers of 28DCR1, 28DCR2, 28DCR3 in the dual-activating chimeric antigen receptor Muc1 CAR-T cells.

FIG. 2A-3: CD3ζ expression profile in the dual-activating chimeric antigen receptor EGFR CAR-T cells. The internal control is GADPH.

FIG. 2B-3: Copy numbers of 40DCR1, 40DCR2, 40DCR3 in the dual-activating chimeric antigen receptor EGFR CAR-T cells.

FIG. 3A-1: Function of electroporation of 137DCR, Mock T proliferation phenotype. The abscissa represents the fluorescence intensity of Hochest positive cells, and the ordinate represents the fluorescence intensity of Ki-67 positive cells. Ki-67 is channel 6, and Hochest is channel 9. The result of co-staining Ki67 and Hochest; after distinguishing between diploid and tetraploid, Ki67 is used to separate G0 phase resting cells and proliferating cells. The first quadrant is the cell undergoing DNA synthesis and division, that is, the cell in the S/G2/M phase; the second quadrant is the preparation phase for division, that is, the G1 phase.

FIG. 3B-1: Function of electroporation of 137DCR1, proliferation phenotype of recombinant cell 137DCR1. The abscissa represents the fluorescence intensity of Hochest positive cells, and the ordinate represents the fluorescence intensity of Ki-67 positive cells. Ki-67 is channel 6, and Hochest is channel 9. The result of co-staining Ki67 and Hochest; after distinguishing between diploid and tetraploid, Ki67 is used to separate G0 phase resting cells and proliferating cells. The first quadrant is the cell undergoing DNA synthesis and division, that is, the cell in the S/G2/M phase; the second quadrant is the preparation phase for division, that is, the G1 phase.

FIG. 3C-1: Function of electroporation of 137DCR2, proliferation phenotype of recombinant cell 137DCR2. The abscissa represents the fluorescence intensity of Hochest positive cells, and the ordinate represents the fluorescence intensity of Ki-67 positive cells. Ki-67 is channel 6, and Hochest is channel 9. The result of co-staining Ki67 and Hochest; after distinguishing between diploid and tetraploid, Ki67 is used to separate G0 phase resting cells and proliferating cells. The first quadrant is the cell undergoing DNA synthesis and division, that is, the cell in the S/G2/M phase; the second quadrant is the preparation phase for division, that is, the G1 phase.

FIG. 3D-1: Function of electroporation of 137DCR3, proliferation phenotype of recombinant cell 137DCR3. The abscissa represents the fluorescence intensity of Hochest positive cells, and the ordinate represents the fluorescence intensity of Ki-67 positive cells. Ki-67 is channel 6, and Hochest is channel 9. The result of co-staining Ki67 and Hochest; after distinguishing between diploid and tetraploid, Ki67 is used to separate G0 phase resting cells and proliferating cells. The first quadrant is the cell undergoing DNA synthesis and division, that is, the cell in the S/G2/M phase; the second quadrant is the preparation phase for division, that is, the G1 phase.

FIG. 3-2: Function of electroporation of 28DCR, proliferation curve of recombinant cell 28DCR1/2/3 cells. The abscissa represents time (h), and the ordinate represents the number of cells.

FIG. 3-3: Function of electroporation of 40DCR, proliferation curve of recombinant cell 40DCR1/2/3 cells. The abscissa represents time (h), and the ordinate represents the number of cells.

FIG. 4-1: Dual-activating chimeric antigen receptor mesothelin CAR-T cells, cell proliferation curve.

FIG. 4-2: Dual-activating chimeric antigen receptor Muc1 CAR-T cells, cell proliferation curve.

FIG. 4-3: Dual-activating chimeric antigen receptor EGFR CAR-T cells, cell proliferation curve.

FIG. 5A-1: Function of electroporation of 137DCR, CD137 phenotype of Mock T. Wherein the abscissa is the fluorescence intensity of a single CD137-positive cell, and the ordinate is the number of cells with different fluorescence intensities.

FIG. 5B-1: Function of electroporation of 137DCR1, CD137 phenotype of recombinant cell 137DCR1. Wherein the abscissa is the fluorescence intensity of a single CD137-positive cell, and the ordinate is the number of cells with different fluorescence intensities.

FIG. 5C-1: Function of electroporation of 137DCR2, CD137 phenotype of recombinant cell 137DCR2. Wherein the abscissa is the fluorescence intensity of a single CD137-positive cell, and the ordinate is the number of cells with different fluorescence intensities.

FIG. 5D-1: Function of electroporation of 137DCR3, CD137 phenotype of recombinant cell 137DCR3. Wherein the abscissa is the fluorescence intensity of a single CD137-positive cell, and the ordinate is the number of cells with different fluorescence intensities.

FIG. 5A-2: Function of electroporation of 28DCR, CD28 phenotype of Mock T. Wherein the abscissa is the fluorescence intensity of a single CD28-positive cell, and the ordinate is the number of cells with different fluorescence intensities.

FIG. 5B-2: Function of electroporation of 28DCR1, CD28 phenotype of recombinant cell 28DCR1. Wherein the abscissa is the fluorescence intensity of a single CD28-positive cell, and the ordinate is the number of cells with different fluorescence intensities.

FIG. 5C-2: Function of electroporation of 28DCR2, CD28 phenotype of recombinant cell 28DCR2. Wherein the abscissa is the fluorescence intensity of a single CD28-positive cell, and the ordinate is the number of cells with different fluorescence intensities.

FIG. 5D-2: Function of electroporation of 28DCR3, CD28 phenotype of recombinant cell 28DCR3. Wherein the abscissa is the fluorescence intensity of a single CD28-positive cell, and the ordinate is the number of cells with different fluorescence intensities.

FIG. 5A-3: Function of electroporation of 40DCR, CD40 phenotype of Mock T. Wherein the abscissa is the fluorescence intensity of a single CD40-positive cell, and the ordinate is the number of cells with different fluorescence intensities.

FIG. 5B-3: Function of electroporation of 40DCR1, CD40 phenotype of recombinant cell 40DCR1. Wherein the abscissa is the fluorescence intensity of a single CD40-positive cell, and the ordinate is the number of cells with different fluorescence intensities.

FIG. 5C-3: Function of electroporation of 40DCR2, CD40 phenotype of recombinant cell 40DCR2. Wherein the abscissa is the fluorescence intensity of a single CD40-positive cell, and the ordinate is the number of cells with different fluorescence intensities.

FIG. 5D-3: Function of electroporation of 40DCR3, CD40 phenotype of recombinant cell 40DCR3. Wherein the abscissa is the fluorescence intensity of a single CD40-positive cell, and the ordinate is the number of cells with different fluorescence intensities.

FIG. 6A-1: Function of electroporation of 137DCR2 combined with meso G1 CAR, CD137 phenotype of Mock T. Wherein the abscissa is the fluorescence intensity of a single CD137-positive cell, and the ordinate is the number of cells with different fluorescence intensities.

FIG. 6B-1: Function of electroporation of 137DCR2 combined with meso G1 CAR, CD137 phenotype of recombinant cell meso G1 CAR. Wherein the abscissa is the fluorescence intensity of a single CD137-positive cell, and the ordinate is the number of cells with different fluorescence intensities.

FIG. 6C-1: Function of electroporation of 137DCR2 combined with meso G1 CAR, CD137 phenotype of recombinant cell meso G2 CAR. Wherein the abscissa is the fluorescence intensity of a single CD137-positive cell, and the ordinate is the number of cells with different fluorescence intensities.

FIG. 6D-1: Function of electroporation of 137DCR combined with meso G1 CAR, CD137 phenotype of recombinant cell meso G1 CAR-137DCR1. Wherein the abscissa is the fluorescence intensity of a single CD137-positive cell, and the ordinate is the number of cells with different fluorescence intensities.

FIG. 6A-2: Function of electroporation of 28DCR2 combined with Muc1 G1 CAR, CD28 phenotype of Mock T. Wherein the abscissa is the fluorescence intensity of a single CD28-positive cell, and the ordinate is the number of cells with different fluorescence intensities.

FIG. 6B-2: Function of electroporation of 28DCR2 combined with Muc1 G1 CAR, CD28 phenotype of recombinant cell Muc1 G1 CAR. Wherein the abscissa is the fluorescence intensity of a single CD28-positive cell, and the ordinate is the number of cells with different fluorescence intensities.

FIG. 6C-2: Function of electroporation of 28DCR2 combined with Muc1 G1 CAR, CD28 phenotype of recombinant cell Muc1 G2 CAR. Wherein the abscissa is the fluorescence intensity of a single CD28-positive cell, and the ordinate is the number of cells with different fluorescence intensities.

FIG. 6D-2: Function of electroporation of 28DCR2 combined with Muc1 G1 CAR, CD28 phenotype of recombinant cell Muc1 G1 CAR-28DCR1. Wherein the abscissa is the fluorescence intensity of a single CD28-positive cell, and the ordinate is the number of cells with different fluorescence intensities.

FIG. 6A-3: Function of electroporation of 40DCR2 combined with EGFR G1 CAR, CD40 phenotype of Mock T. Wherein the abscissa is the fluorescence intensity of a single CD40-positive cell, and the ordinate is the number of cells with different fluorescence intensities.

FIG. 6B-3: Function of electroporation of 40DCR2 combined with EGFR G1 CAR, CD40 phenotype of recombinant cell EGFR G1 CAR. Wherein the abscissa is the fluorescence intensity of a single CD40-positive cell, and the ordinate is the number of cells with different fluorescence intensities.

FIG. 6C-3: Function of electroporation of 40DCR2 combined with EGFR G1 CAR, CD40 phenotype of recombinant cell EGFR G2 CAR. Wherein the abscissa is the fluorescence intensity of a single CD40-positive cell, and the ordinate is the number of cells with different fluorescence intensities.

FIG. 6D-3: Function of electroporation of 40DCR2 combined with EGFR G1 CAR, CD40 phenotype of recombinant cell EGFR G1 CAR-40DCR1. Wherein the abscissa is the fluorescence intensity of a single CD40-positive cell, and the ordinate is the number of cells with different fluorescence intensities.

FIG. 7A-1: Function of electroporation of 137DCR2 combined with meso G1 CAR, CD45RO phenotype of Mock T. Wherein the abscissa is the fluorescence intensity of a single CD45RO-positive cell, and the ordinate is the number of cells with different fluorescence intensities.

FIG. 7B-1: Function of electroporation of 137DCR2 combined with meso G1 CAR, CD45RO phenotype of recombinant cell meso G1 CAR. Wherein the abscissa is the fluorescence intensity of a single CD45RO-positive cell, and the ordinate is the number of cells with different fluorescence intensities.

FIG. 7C-1: Function of electroporation of 137DCR2 combined with meso G1 CAR, CD45RO phenotype of recombinant cell meso G2 CAR. Wherein the abscissa is the fluorescence intensity of a single CD45RO-positive cell, and the ordinate is the number of cells with different fluorescence intensities.

FIG. 7D-1: Function of electroporation of 137DCR combined with meso G1 CAR, CD45RO phenotype of recombinant cell meso G1 CAR-137DCR1. Wherein the abscissa is the fluorescence intensity of a single CD45RO-positive cell, and the ordinate is the number of cells with different fluorescence intensities.

FIG. 7A-2: Function of electroporation of 28DCR2 combined with Muc1 G1 CAR, CD45RO phenotype of Mock T. Wherein the abscissa is the fluorescence intensity of a single CD45RO-positive cell, and the ordinate is the number of cells with different fluorescence intensities.

FIG. 7B-2: Function of electroporation of 28DCR2 combined with Muc1 G1 CAR, CD45RO phenotype of recombinant cell Muc1 G1 CAR. Wherein the abscissa is the fluorescence intensity of a single CD45RO-positive cell, and the ordinate is the number of cells with different fluorescence intensities.

FIG. 7C-2: Function of electroporation of 28DCR2 combined with Muc1 G1 CAR, CD45RO phenotype of recombinant cell Muc1 G2 CAR. Wherein the abscissa is the fluorescence intensity of a single CD45RO-positive cell, and the ordinate is the number of cells with different fluorescence intensities.

FIG. 7D-2: Function of electroporation of 28DCR combined with Muc1 G1 CAR, CD45RO phenotype of recombinant cell Muc1 G1 CAR-28DCR1. Wherein the abscissa is the fluorescence intensity of a single CD45RO-positive cell, and the ordinate is the number of cells with different fluorescence intensities.

FIG. 7A-3: Function of electroporation of 40DCR2 combined with EGFR G1 CAR, CD45RO phenotype of Mock T. Wherein the abscissa is the fluorescence intensity of a single CD45RO-positive cell, and the ordinate is the number of cells with different fluorescence intensities.

FIG. 7B-3: Function of electroporation of 40DCR2 combined with EGFR G1 CAR, CD45RO phenotype of recombinant cell EGFR G1 CAR. Wherein the abscissa is the fluorescence intensity of a single CD45RO-positive cell, and the ordinate is the number of cells with different fluorescence intensities.

FIG. 7C-3: Function of electroporation of 40DCR2 combined with EGFR G1 CAR, CD45RO phenotype of recombinant cell EGFR G2 CAR. Wherein the abscissa is the fluorescence intensity of a single CD45RO-positive cell, and the ordinate is the number of cells with different fluorescence intensities.

FIG. 7D-3: Function of electroporation of 40DCR2 combined with EGFR G1 CAR, CD45RO phenotype of recombinant cell EGFR G1 CAR-40DCR1. Wherein the abscissa is the fluorescence intensity of a single CD45RO-positive cell, and the ordinate is the number of cells with different fluorescence intensities.

FIG. 8A-1: Function of electroporation of 137DCR2 combined with meso G1 CAR, memory T phenotype of Mock T. Wherein the abscissa is the fluorescence intensity of a single CD62L-positive cell, and the ordinate is the fluorescence intensity of a single CCR7-positive cell.

FIG. 8B-1: Function of electroporation of 137DCR2 combined with meso G1 CAR, memory T phenotype of recombinant cell meso G1 CAR. Wherein the abscissa is the fluorescence intensity of a single CD62L-positive cell, and the ordinate is the fluorescence intensity of a single CCR7-positive cell.

FIG. 8C-1: Function of electroporation of 137DCR2 combined with meso G1 CAR, memory T phenotype of recombinant cell meso G2 CAR. Wherein the abscissa is the fluorescence intensity of a single CD62L-positive cell, and the ordinate is the fluorescence intensity of a single CCR7-positive cell.

FIG. 8D-1: Function of electroporation of 137DCR combined with meso G1 CAR, memory T phenotype of recombinant cell meso G1 CAR-137DCR1. Wherein the abscissa is the fluorescence intensity of a single CD62L-positive cell, and the ordinate is the fluorescence intensity of a single CCR7-positive cell.

FIG. 8A-2: Function of electroporation of 28DCR2 combined with Muc1 G1 CAR, memory T phenotype of Mock T. Wherein the abscissa is the fluorescence intensity of a single CD62L-positive cell, and the ordinate is the fluorescence intensity of a single CCR7-positive cell.

FIG. 8B-2: Function of electroporation of 28DCR2 combined with Muc1 G1 CAR, memory T phenotype of recombinant cell Muc1 G1 CAR. Wherein the abscissa is the fluorescence intensity of a single CD62L-positive cell, and the ordinate is the fluorescence intensity of a single CCR7-positive cell.

FIG. 8C-2: Function of electroporation of 28DCR2 combined with Muc1 G1 CAR, memory T phenotype of recombinant cell Muc1 G2 CAR. Wherein the abscissa is the fluorescence intensity of a single CD62L-positive cell, and the ordinate is the fluorescence intensity of a single CCR7-positive cell.

FIG. 8D-2: Function of electroporation of 28DCR combined with Muc1 G1 CAR, memory T phenotype of recombinant cell Muc1 G1 CAR-28DCR1. Wherein the abscissa is the fluorescence intensity of a single CD62L-positive cell, and the ordinate is the fluorescence intensity of a single CCR7-positive cell.

FIG. 8A-3: Function of electroporation of 40DCR2 combined with EGFR G1 CAR, memory T phenotype of Mock T. Wherein the abscissa is the fluorescence intensity of a single CD62L-positive cell, and the ordinate is the fluorescence intensity of a single CCR7-positive cell.

FIG. 8B-3: Function of electroporation of 40DCR2 combined with EGFR G1 CAR, memory T phenotype of recombinant cell EGFR G1 CAR. Wherein the abscissa is the fluorescence intensity of a single CD62L-positive cell, and the ordinate is the fluorescence intensity of a single CCR7-positive cell.

FIG. 8C-3: Function of electroporation of 40DCR2 combined with EGFR G1 CAR, memory T phenotype of recombinant cell EGFR G2 CAR. Wherein the abscissa is the fluorescence intensity of a single CD62L-positive cell, and the ordinate is the fluorescence intensity of a single CCR7-positive cell.

FIG. 8D-3: Function of electroporation of 40DCR combined with EGFR G1 CAR, memory T phenotype of recombinant cell EGFR G1 CAR-40DCR1. Wherein the abscissa is the fluorescence intensity of a single CD62L-positive cell, and the ordinate is the fluorescence intensity of a single CCR7-positive cell.

FIG. 9A-1: Dual-activating chimeric antigen receptor mesothelin CAR-T cells kill Hela tumor cell lines in vitro with an effector target ratio of 8:1.

FIG. 9B-1: Dual-activating chimeric antigen receptor mesothelin CAR-T cells kill Hela tumor cell lines in vitro with an effector target ratio of 4:1.

FIG. 9C-1: Dual-activating chimeric antigen receptor mesothelin CAR-T cells kill SK-OV-3 tumor cell lines in vitro with an effector target ratio of 8:1.

FIG. 9D-1: Dual-activating chimeric antigen receptor mesothelin CAR-T cells kill SK-OV-3 tumor cell lines in vitro with an effector target ratio of 4:1.

FIG. 9A-2: Dual-activating chimeric antigen receptor Muc1 CAR-T cells kill MCF7 tumor cell lines in vitro with an effector target ratio of 8:1.

FIG. 9B-2: Dual-activating chimeric antigen receptor Muc1 CAR-T cells kill MCF7 tumor cell lines in vitro with an effector target ratio of 4:1.

FIG. 9C-2: Dual-activating chimeric antigen receptor Muc1 CAR-T cells kill A549 tumor cell lines in vitro with an effector target ratio of 8:1.

FIG. 9D-2: Dual-activating chimeric antigen receptor Muc1 CAR-T cells kill A549 tumor cell lines in vitro with an effector target ratio of 4:1.

FIG. 9A-3: Dual-activating chimeric antigen receptor EGFR CAR-T cells kill H23 tumor cell lines in vitro with an effector target ratio of 8:1.

FIG. 9B-3: Dual-activating chimeric antigen receptor EGFR CAR-T cells kill H23 tumor cell lines in vitro with an effector target ratio of 4:1.

FIG. 9C-3: Dual-activating chimeric antigen receptor EGFR CAR-T cells kill ASPC-1 tumor cell lines in vitro with an effector target ratio of 8:1.

FIG. 9D-3: Dual-activating chimeric antigen receptor EGFR CAR-T cells kill ASPC-1 tumor cell lines in vitro with an effector target ratio of 4:1.

FIG. 10-1: Changes in cytokines IL-2, IL-4, IL-6, IL-10, TNF-α and IFN-γ of dual-activating chimeric antigen receptor mesothelin CAR-T cells under the stimulation with mesothelin antigen.

FIG. 10-2: Changes in cytokines IL-2, IL-4, IL-6, IL-10, TNF-α and IFN-γ of dual-activating chimeric antigen receptor Muc1 CAR-T cells under the stimulation with Muc1 antigen.

FIG. 10-3: Changes in cytokines IL-2, IL-4, IL-6, IL-10, TNF-α and IFN-γ of dual-activating chimeric antigen receptor EGFR CAR-T cells under the stimulation with EGFR antigen.

FIG. 11-1: The therapeutic effect of dual-activating chimeric antigen receptor mesothelin CAR-T cells on an ovarian cancer xenograft mouse model.

FIG. 11-2: The therapeutic effect of dual-activating chimeric antigen receptor Muc1 CAR-T cells on an ovarian cancer xenograft mouse model.

FIG. 11-3: The therapeutic effect of dual-activating chimeric antigen receptor EGFR CAR-T cells on an ovarian cancer xenograft mouse model.

Some sequences involved in the present invention are as follows:

1. Amino acid sequence of CDS signal peptide
(SEQ ID NO: 1)
MGNSCYNIVATLLLVLNFERTRS
2. Amino acid sequence of CD 137 extracellular agonistic
single-chain antibody
(SEQ ID NO: 2)
QVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGT
LVTVSSGGGGSGGGGSGGGGSEIVLTQSPATLSLSPGERATLSCRASQSVSSYLAWYQQK
PGQAPRLLIYDASNRATGIPARFSGSGSGTDFTLTISSLEPEDFAVYYCQQRSNWPPALTFG
GGTKVEIKE
3. Amino acid sequence of CD8α hinge region
(SEQ ID NO: 3)
SKYGPPCPPCPIEVMYPPPYLDNEKSNGTIIHVKGKHLCPSPLFPGPSK
4. Amino acid sequence of CD28 transmembrane region
(SEQ ID NO: 4)
PFWVLVVVGGVLACYSLLVTVAFIIFWV
5. Amino acid sequence of CD28 intracellular costimulatory
signal domain
(SEQ ID NO: 5)
RSKRSRLLHSDYMNMTPRRPGPTRKHYQPYAPPRDFAAYRS
6. Amino acid sequence of CD137 intracellular costimulatory
signal domain
(SEQ ID NO: 6)
KRGRKKLLYIFKQPFMRPVQTTQEEDGCSCRFPEEEEGGCEL
7. Amino acid sequence of 137DCR1 (including signal peptide)
(SEQ ID NO: 7)
MGNSCYNIVATLLLVLNFERTRSQVQLQQWGAGLLKPSETLSLTCAVYGGSFSGYY
WSWIRQSPEKGLEWIGEINHGGYVTYNPSLESRVTISVDTSKNQFSLKLSSVTAADTAVY
YCARDYGPGNYDWYFDLWGRGTLVTVSSGGGGSGGGGSGGGGSEIVLTQSPATLSLSPG
ERATLSCRASQSVSSYLAWYQQKPGQAPRLLIYDASNRATGIPARFSGSGSGTDFTLTISSL
EPEDFAVYYCQQRSNWPPALTFGGGTKVEIRESKYGPPCPPCPIEVMYPPPYLDNEKSNGT
IIHVKGKHLCPSPLFPGPSKPFWVLVVVGGVLACYSLLVTVAFIIFWVRSKRSRLLHSDYM
NMTPRRPGPTRKHYQPYAPPRDFAAYRSS
Amino acid sequence of 137DCR2 (including signal peptide)
(SEQ ID NO: 8)
MGNSCYNIVATLLLVLNFERTRSQVQLQQWGAGLLKPSETLSLTCAVYGGSFSGYY
WSWIRQSPEKGLEWIGEINHGGYVTYNPSLESRVTISVDTSKNQFSLKLSSVTAADTAVY
YCARDYGPGNYDWYFDLWGRGTLVTVSSGGGGSGGGGSGGGGSEIVLTQSPATLSLSPG
ERATLSCRASQSVSSYLAWYQQKPGQAPRLLIYDASNRATGIPARFSGSGSGTDFTLTISSL
EPEDFAVYYCQQRSNWPPALTFGGGTKVEIKESKYGPPCPPCPIEVMYPPPYLDNEKSNGT
IIHVKGKHLCPSPLFPGPSKPFWVLVVVGGVLACYSLLVTVAFIIFWVKRGRKKLLYIFKQ
PFMRPVQTTQEEDGCSCRFPEEEEGGCEL
9. Amino acid sequence of 137DCR3 (including signal peptide)
(SEQ ID NO: 9)
MGNSCYNIVATLLLVLNFERTRSQVQLQQWGAGLLKPSETLSLTCAVYGGSFSGYY
WSWIRQSPEKGLEWIGEINHGGYVTYNPSLESRVTISVDTSKNQFSLKLSSVTAADTAVY
YCARDYGPGNYDWYFDLWGRGTLVTVSSGGGGSGGGGSGGGGSEIVLTQSPATLSLSPG
ERATLSCRASQSVSSYLAWYQQKPGQAPRLLIYDASNRATGIPARFSGSGSGTDFTLTISSL
EPEDFAVYYCQQRSNWPPALTFGGGTKVEIKESKYGPPCPPCPIEVMYPPPYLDNEKSNGT
IIHVKGKHLCPSPLFPGPSKPFWVLVVVGGVLACYSLLVTVAFIIFWVRSKRSRLLHSDYM
NMTPRRPGPTRKHYQPYAPPRDFAAYRSKRGRKKLLYIFKQPFMRPVQTTQEEDGCSCRF
PEEEEGGCEL
10. Amino acid sequence of 137DCR4 (including signal peptide)
(SEQ ID NO: 10)
MGNSCYNIVATLLLVLNFERTRSQVQLQQWGAGLLKPSETLSLTCAVYGGSFSGYY
WSWIRQSPEKGLEWIGEINHGGYVTYNPSLESRVTISVDTSKNQFSLKLSSVTAADTAVY
YCARDYGPGNYDWYFDLWGRGTLVTVSSGGGGSGGGGSGGGGSEIVLTQSPATLSLSPG
ERATLSCRASQSVSSYLAWYQQKPGQAPRLLIYDASNRATGIPARFSGSGSGTDFTLTISSL
EPEDFAVYYCQQRSNWPPALTFGGGTKVEIKESKYGPPCPPCPIEVMYPPPYLDNEKSNGT
IIHVKGKHLCPSPLFPGPSKPFWVLVVVGGVLACYSLLVTVAFIIFWVKRGRKKLLYIFKQ
PFMRPVQTTQEEDGCSCRFPEEEEGGCELRSKRSRLLHSDYMNMTPRRPGPTRKHYQPYA
PPRDFAAYRS
11. Amino acid sequence of 137DCR1 (without signal peptide)
(SEQ ID NO: 11)
QVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGT
LVTVSSGGGGSGGGGSGGGGSEIVLTQSPATLSLSPGERATLSCRASQSVSSYLAWYQQK
PGQAPRLLIYDASNRATGIPARFSGSGSGTDFTLTISSLEPEDFAVYYCQQRSNWPPALTFG
GGTKVEIKESKYGPPCPPCPIEVMYPPPYLDNEKSNGTIIHVKGKHLCPSPLFPGPSKPFWV
LVVVGGVLACYSLLVTVAFIIFWVRSKRSRLLHSDYMNMTPRRPGPTRKHYQPYAPPRDF
AAYRS
12. Amino acid sequence of 137DCR2 (without signal peptide)
(SEQ ID NO: 12)
QVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGT
LVTVSSGGGGSGGGGSGGGGSEIVLTQSPATLSLSPGERATLSCRASQSVSSYLAWYQQK
PGQAPRLLIYDASNRATGIPARFSGSGSGTDFTLTISSLEPEDFAVYYCQQRSNWPPALTFG
GGTKVEIKESKYGPPCPPCPIEVMYPPPYLDNEKSNGTUHVKGKHLCPSPLFPGPSKPFWV
LVVVGGVLACYSLLVTVAFIIFWVKRGRKKLLYIFKQPFMRPVQTTQEEDGCSCRFPEEEE
GGCEL
13. Amino acid sequence of 137DCR3 (without signal peptide)
(SEQ ID NO: 13)
QVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGT
LVTVSSGGGGSGGGGSGGGGSEIVLTQSPATLSLSPGERATLSCRASQSVSSYLAWYQQK
PGQAPRLLIYDASNRATGIPARFSGSGSGTDFTLTISSLEPEDFAVYYCQQRSNWPPALTFG
GGTKVEIKESKYGPPCPPCPIEVMYPPPYLDNEKSNGTIIHVKGKHLCPSPLFPGPSKPFWV
LVVVGGVLACYSLLVTVAFIIFWVRSKRSRLLHSDYMNMTPRRPGPTRKHYQPYAPPRDF
AAYRSKRGRKKLLYIFKQPFMRPVQTTQEEDGCSCRFPEEEEGGCEL
14. Amino acid sequence of 137DCR4 (without signal peptide)
(SEQ ID NO: 14)
QVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGT
LVTVSSGGGGSGGGGSGGGGSEIVLTQSPATLSLSPGERATLSCRASQSVSSYLAWYQQK
PGOAPRLLIYDASNRATGIPARFSGSGSGTDFTLTISSLEPEDFAVYYCQQRSNWPPALTFG
GGTKVEIKESKYGPPCPPCPIEVMYPPPYLDNEKSNGTIIHVKGKHLCPSPLFPGPSKPFWV
LVVVGGVLACYSLLVTVAFIIFWVKRGRKKLLYIFKQPFMRPVQTTQEEDGCSCRFPEEEE
GGCELRSKRSRLLHSDYMNMTPRRPGPTRKHYQPYAPPRDFAAYRS
15. Nucleic acid sequence of 137DCR1 (including signal peptide)
(SEQ ID NO: 15)
ATGGGAAACAGCTGTTACAACATAGTAGCCACTCTGTTGCTGGTCCTCAACTTTG
AGAGGACAAGATCACAGGTGCAACTACAGCAGTGGGGCGCAGGACTGTTGAAGCCTT
CGGAGACCCTGTCCCTCACCTGCGCTGTCTATGGTGGGTCCTTCAGTGGTTACTACTG
GAGCTGGATACGCCAGTCCCCAGAGAAGGGGCTGGAGTGGATTGGGGAAATCAATCA
TGGTGGATACGTCACCTACAATCCGTCCCTCGAGAGTCGAGTCACCATATCAGTAGAC
ACGTCCAAGAACCAGTTCTCCCTGAAGCTGAGCTCTGTGACCGCCGCGGACACGGCT
GTATATTACTGTGCGAGGGACTATGGTCCGGGGAATTATGACTGGTACTTCGATCTCT
GGGGCCGTGGCACCCTGGTCACTGTCTCCTCAGGTGGAGGCGGTTCAGGCGGAGGTG
GCAGCGGCGGTGGCGGGTCGGAAATTGTGTTGACACAGTCTCCAGCCACCCTGTCTTT
GTCTCCAGGGGAAAGAGCCACCCTCTCCTGCAGGGCCAGTCAGAGTGTTAGCAGCTA
CTTAGCCTGGTACCAACAGAAACCTGGCCAGGCTCCCAGGCTCCTCATCTATGATGCA
TCCAACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGTCAGC
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCA
GCCTATCGCTCCTGATAA
16. Nucleic acid sequence of 137DCR2 (including signal peptide)
(SEQ ID NO: 16)
ATGGGAAACAGCTGTTACAACATAGTAGCCACTCTGTTGCTGGTCCTCAACTTTG
AGAGGACAAGATCACAGGTGCAACTACAGCAGTGGGGCGCAGGACTGTTGAAGCCTT
CGGAGACCCTGTCCCTCACCTGCGCTGTCTATGGTGGGTCCTTCAGTGGTTACTACTG
GAGCTGGATACGCCAGTCCCCAGAGAAGGGGCTGGAGTGGATTGGGGAAATCAATCA
TGGTGGATACGTCACCTACAATCCGTCCCTCGAGAGTCGAGTCACCATATCAGTAGAC
ACGTCCAAGAACCAGTTCTCCCTGAAGCTGAGCTCTGTGACCGCCGCGGACACGGCT
GTATATTACTGTGCGAGGGACTATGGTCCGGGGAATTATGACTGGTACTTCGATCTCT
GGGGCCGTGGCACCCTGGTCACTGTCTCCTCAGGTGGAGGCGGTTCAGGCGGAGGTG
GCAGCGGCGGTGGCGGGTCGGAAATTGTGTTGACACAGTCTCCAGCCACCCTGTCTTT
GTCTCCAGGGGAAAGAGCCACCCTCTCCTGCAGGGCCAGTCAGAGTGTTAGCAGCTA
CTTAGCCTGGTACCAACAGAAACCTGGCCAGGCTCCCAGGCTCCTCATCTATGATGCA
TCCAACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGTCAGC
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAAACGGGGCAGAAAGAAGCTCCTGTATATATTCAAACAACCATTTATGAGACCA
GTACAAACTACTCAAGAGGAAGATGGCTGTAGCTGCCGATTTCCAGAAGAAGAAGAA
GGAGGATGTGAACTGTGATAA
17. Nucleic acid sequence of 137DCR3 (including signal peptide)
(SEQ ID NO: 17)
ATGGGAAACAGCTGTTACAACATAGTAGCCACTCTGTTGCTGGTCCTCAACTTTG
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCA
GCCT ATCGCTCCTGATAA
16. Nucleic acid sequence of 137DCR2 (including signal peptide)
(SEQ ID NO: 16)
ATGGGAAACAGCTGTTACAACATAGTAGCCACTCTGTTGCTGGTCCTCAACTTTG
AGAGGACAAGATCACAGGTGCAACTACAGCAGTGGGGCGCAGGACTGTTGAAGCCTT
CGGAGACCCTGTCCCTCACCTGCGCTGTCTATGGTGGGTCCTTCAGTGGTTACTACTG
GAGCTGGATACGCCAGTCCCCAGAGAAGGGGCTGGAGTGGATTGGGGAAATCAATCA
TGGTGGATACGTCACCTACAATCCGTCCCTCGAGAGTCGAGTCACCATATCAGTAGAC
ACGTCCAAGAACCAGTTCTCCCTGAAGCTGAGCTCTGTGACCGCCGCGGACACGGCT
GTATATTACTGTGCGAGGGACTATGGTCCGGGGAATTATGACTGGTACTTCGATCTCT
GGGGCCGTGGCACCCTGGTCACTGTCTCCTCAGGTGGAGGCGGTTCAGGCGGAGGTG
GCAGCGGCGGTGGCGGGTCGGAAATTGTGTTGACACAGTCTCCAGCCACCCTGTCTTT
GTCTCCAGGGGAAAGAGCCACCCTCTCCTGCAGGGCCAGTCAGAGTGTTAGCAGCTA
CTTAGCCTGGTACCAACAGAAACCTGGCCAGGCTCCCAGGCTCCTCATCTATGATGCA
TCCAACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGTCAGC
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAAACGGGGCAGAAAGAAGCTCCTGTATATATTCAAACAACCATTTATGAGACCA
GTACAAACTACTCAAGAGGAAGATGGCTGTAGCTGCCGATTTCCAGAAGAAGAAGAA
GGAGGATGTGAACTGTGATAA
17. Nucleic acid sequence of 137DCR3 (including signal peptide)
(SEQ ID NO: 17)
ATGGGAAACAGCTGTTACAACATAGTAGCCACTCTGTTGCTGGTCCTCAACTTTG
AGAGGACAAGATCACAGGTGCAACTACAGCAGTGGGGCGCAGGACTGTTGAAGCCTT
CGGAGACCCTGTCCCTCACCTGCGCTGTCTATGGTGGGTCCTTCAGTGGTTACTACTG
GAGCTGGATACGCCAGTCCCCAGAGAAGGGGCTGGAGTGGATTGGGGAAATCAATCA
TGGTGGATACGTCACCTACAATCCGTCCCTCGAGAGTCGAGTCACCATATCAGTAGAC
ACGTCCAAGAACCAGTTCTCCCTGAAGCTGAGCTCTGTGACCGCCGCGGACACGGCT
GTATATTACTGTGCGAGGGACTATGGTCCGGGGAATTATGACTGGTACTTCGATCTCT
GGGGCCGTGGCACCCTGGTCACTGTCTCCTCAGGTGGAGGCGGTTCAGGCGGAGGTG
GCAGCGGCGGTGGCGGGTCGGAAATTGTGTTGACACAGTCTCCAGCCACCCTGTCTTT
GTCTCCAGGGGAAAGAGCCACCCTCTCCTGCAGGGCCAGTCAGAGTGTTAGCAGCTA
CTTAGCCTGGTACCAACAGAAACCTGGCCAGGCTCCCAGGCTCCTCATCTATGATGCA
TCCAACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGTCAGC
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCA
GCCTATCGCTCCAAACGGGGCAGAAAGAAGCTCCTGTATATATTCAAACAACCATTT
ATGAGACCAGTACAAACTACTCAAGAGGAAGATGGCTGTAGCTGCCGATTTCCAGAA
GAAGAAGAAGGAGGATGTGAACTGTGATAA
18. Nucleic acid sequence of 137DCR4 (including signal peptide)
(SEQ ID NO: 18)
ATGGGAAACAGCTGTTACAACATAGTAGCCACTCTGTTGCTGGTCCTCAACTTTG
AGAGGACAAGATCACAGGTGCAACTACAGCAGTGGGGCGCAGGACTGTTGAAGCCTT
CGGAGACCCTGTCCCTCACCTGCGCTGTCTATGGTGGGTCCTTCAGTGGTTACTACTG
GAGCTGGATACGCCAGTCCCCAGAGAAGGGGCTGGAGTGGATTGGGGAAATCAATCA
TGGTGGATACGTCACCTACAATCCGTCCCTCGAGAGTCGAGTCACCATATCAGTAGAC
ACGTCCAAGAACCAGTTCTCCCTGAAGCTGAGCTCTGTGACCGCCGCGGACACGGCT
GTATATTACTGTGCGAGGGACTATGGTCCGGGGAATTATGACTGGTACTTCGATCTCT
GGGGCCGTGGCACCCTGGTCACTGTCTCCTCAGGTGGAGGCGGTTCAGGCGGAGGTG
GCAGCGGCGGTGGCGGGTCGGAAATTGTGTTGACACAGTCTCCAGCCACCCTGTCTTT
GTCTCCAGGGGAAAGAGCCACCCTCTCCTGCAGGGCCAGTCAGAGTGTTAGCAGCTA
CTTAGCCTGGTACCAACAGAAACCTGGCCAGGCTCCCAGGCTCCTCATCTATGATGCA
TCCAACAGGGCCACTGGCATCCCAGCCAGGTTCAGTGGCAGTGGGTCTGGGACAGAC
TTCACTCTCACCATCAGCAGCCTAGAGCCTGAAGATTTTGCAGTTTATTACTGTCAGC
AGCGTAGCAACTGGCCTCCGGCGCTCACTTTCGGCGGAGGGACCAAGGTGGAGATCA
AAGAGTCCAAATATGGTCCCCCATGCCCACCATGCCCAATTGAAGTTATGTATCCTCC
TCCTTACCTAGACAATGAGAAGAGCAATGGAACCATTATCCATGTGAAAGGGAAACA
CCTTTGTCCAAGTCCCCTATTTCCCGGACCTTCTAAGCCCTTTTGGGTGCTGGTGGTGG
TTGGTGGAGTCCTGGCTTGCTATAGCTTGCTAGTAACAGTGGCCTTTATTATTTTCTGG
GTGAAACGGGGCAGAAAGAAGCTCCTGTATATATTCAAACAACCATTTATGAGACCA
GTACAAACTACTCAAGAGGAAGATGGCTGTAGCTGCCGATTTCCAGAAGAAGAAGAA
GGAGGATGTGAACTGAGGAGTAAGAGGAGCAGGCTCCTGCACAGTGACTACATGAA
CATGACTCCCCGCCGCCCCGGGCCCACCCGCAAGCATTACCAGCCCTATGCCCCACCA
CGCGACTTCGCAGCCTATCGCTCCTGATAA
19. Nucleic acid sequence of 137DCR1 (without signal peptide)
(SEQ ID NO: 19)
CAGGTGCAACTACAGCAGTGGGGCGCAGGACTGTTGAAGCCTTCGGAGACCCTG
TCCCTCACCTGCGCTGTCTATGGTGGGTCCTTCAGTGGTTACTACTGGAGCTGGATAC
GCCAGTCCCCAGAGAAGGGGCTGGAGTGGATTGGGGAAATCAATCATGGTGGATACG
TCACCTACAATCCGTCCCTCGAGAGTCGAGTCACCATATCAGTAGACACGTCCAAGA
ACCAGTTCTCCCTGAAGCTGAGCTCTGTGACCGCCGCGGACACGGCTGTATATTACTG
TGCGAGGGACTATGGTCCGGGGAATTATGACTGGTACTTCGATCTCTGGGGCCGTGGC
ACCCTGGTCACTGTCTCCTCAGGTGGAGGCGGTTCAGGCGGAGGTGGCAGCGGCGGT
GGCGGGTCGGAAATTGTGTTGACACAGTCTCCAGCCACCCTGTCTTTGTCTCCAGGGG
AAAGAGCCACCCTCTCCTGCAGGGCCAGTCAGAGTGTTAGCAGCTACTTAGCCTGGT
ACCAACAGAAACCTGGCCAGGCTCCCAGGCTCCTCATCTATGATGCATCCAACAGGG
CCACTGGCATCCCAGCCAGGTTCAGTGGCAGTGGGTCTGGGACAGACTTCACTCTCAC
CATCAGCAGCCTAGAGCCTGAAGATTTTGCAGTTTATTACTGTCAGCAGCGTAGCAAC
TGGCCTCCGGCGCTCACTTTCGGCGGAGGGACCAAGGTGGAGATCAAAGAGTCCAAA
TATGGTCCCCCATGCCCACCATGCCCAATTGAAGTTATGTATCCTCCTCCTTACCTAGA
CAATGAGAAGAGCAATGGAACCATTATCCATGTGAAAGGGAAACACCTTTGTCCAAG
TCCCCTATTTCCCGGACCTTCTAAGCCCTTTTGGGTGCTGGTGGTGGTTGGTGGAGTCC
TGGCTTGCTATAGCTTGCTAGTAACAGTGGCCTTTATTATTTTCTGGGTGAGGAGTAA
GAGGAGCAGGCTCCTGCACAGTGACTACATGAACATGACTCCCCGCCGCCCCGGGCC
CACCCGCAAGCATTACCAGCCCTATGCCCCACCACGCGACTTCGCAGCCTATCGCTCC
TGATAA
20. Nucleic acid sequence of 137DCR2 (without signal peptide)
(SEQ ID NO: 20)
CAGGTGCAACTACAGCAGTGGGGCGCAGGACTGTTGAAGCCTTCGGAGACCCTG
TCCCTCACCTGCGCTGTCTATGGTGGGTCCTTCAGTGGTTACTACTGGAGCTGGATAC
GCCAGTCCCCAGAGAAGGGGCTGGAGTGGATTGGGGAAATCAATCATGGTGGATACG
TCACCTACAATCCGTCCCTCGAGAGTCGAGTCACCATATCAGTAGACACGTCCAAGA
ACCAGTTCTCCCTGAAGCTGAGCTCTGTGACCGCCGCGGACACGGCTGTATATTACTG
TGCGAGGGACTATGGTCCGGGGAATTATGACTGGTACTTCGATCTCTGGGGCCGTGGC
ACCCTGGTCACTGTCTCCTCAGGTGGAGGCGGTTCAGGCGGAGGTGGCAGCGGCGGT
GGCGGGTCGGAAATTGTGTTGACACAGTCTCCAGCCACCCTGTCTTTGTCTCCAGGGG
AAAGAGCCACCCTCTCCTGCAGGGCCAGTCAGAGTGTTAGCAGCTACTTAGCCTGGT
ACCAACAGAAACCTGGCCAGGCTCCCAGGCTCCTCATCTATGATGCATCCAACAGGG
CCACTGGCATCCCAGCCAGGTTCAGTGGCAGTGGGTCTGGGACAGACTTCACTCTCAC
CATCAGCAGCCTAGAGCCTGAAGATTTTGCAGTTTATTACTGTCAGCAGCGTAGCAAC
TGGCCTCCGGCGCTCACTTTCGGCGGAGGGACCAAGGTGGAGATCAAAGAGTCCAAA
TATGGTCCCCCATGCCCACCATGCCCAATTGAAGTTATGTATCCTCCTCCTTACCTAGA
CAATGAGAAGAGCAATGGAACCATTATCCATGTGAAAGGGAAACACCTTTGTCCAAG
TCCCCTATTTCCCGGACCTTCTAAGCCCTTTTGGGTGCTGGTGGTGGTTGGTGGAGTCC
TGGCTTGCTATAGCTTGCTAGTAAC AGTGGCCTTTATTATTTTCTGGGTGAAACGGGG
CAGAAAGAAGCTCCTGTATATATTCAAACAACCATTTATGAGACCAGTACAAACTAC
TCAAGAGGAAGATGGCTGTAGCTGCCGATTTCCAGAAGAAGAAGAAGGAGGATGTG
AACTGTGATAA
21. Nucleic acid sequence of 137DCR3 (without signal peptide)
(SEQ ID NO: 21)
CAGGTGCAACTACAGCAGTGGGGCGCAGGACTGTTGAAGCCTTCGGAGACCCTG
TCCCTCACCTGCGCTGTCTATGGTGGGTCCTTCAGTGGTTACTACTGGAGCTGGATAC
GCCAGTCCCCAGAGAAGGGGCTGGAGTGGATTGGGGAAATCAATCATGGTGGATACG
TCACCTACAATCCGTCCCTCGAGAGTCGAGTCACCATATCAGTAGACACGTCCAAGA
ACCAGTTCTCCCTGAAGCTGAGCTCTGTGACCGCCGCGGACACGGCTGTATATTACTG
TGCGAGGGACTATGGTCCGGGGAATTATGACTGGTACTTCGATCTCTGGGGCCGTGGC
ACCCTGGTCACTGTCTCCTCAGGTGGAGGCGGTTCAGGCGGAGGTGGCAGCGGCGGT
GGCGGGTCGGAAATTGTGTTGACACAGTCTCCAGCCACCCTGTCTTTGTCTCCAGGGG
AAAGAGCCACCCTCTCCTGCAGGGCCAGTCAGAGTGTTAGCAGCTACTTAGCCTGGT
ACCAACAGAAACCTGGCCAGGCTCCCAGGCTCCTCATCTATGATGCATCCAACAGGG
CCACTGGCATCCCAGCCAGGTTCAGTGGCAGTGGGTCTGGGACAGACTTCACTCTCAC
CATCAGCAGCCTAGAGCCTGAAGATTTTGCAGTTTATTACTGTCAGCAGCGTAGCAAC
TGGCCTCCGGCGCTCACTTTCGGCGGAGGGACCAAGGTGGAGATCAAAGAGTCCAAA
TATGGTCCCCCATGCCCACCATGCCCAATTGAAGTTATGTATCCTCCTCCTTACCTAGA
CAATGAGAAGAGCAATGGAACCATTATCCATGTGAAAGGGAAACACCTTTGTCCAAG
TCCCCTATTTCCCGGACCTTCTAAGCCCTTTTGGGTGCTGGTGGTGGTTGGTGGAGTCC
TGGCTTGCTATAGCTTGCTAGTAAC AGTGGCCTTTATTATTTTCTGGGTGAGGAGTAA
GAGGAGCAGGCTCCTGCACAGTGACTACATGAACATGACTCCCCGCCGCCCCGGGCC
CACCCGCAAGCATTACCAGCCCTATGCCCCACCACGCGACTTCGCAGCCTATCGCTCC
AAACGGGGCAGAAAGAAGCTCCTGTATATATTCAAACAACCATTTATGAGACCAGTA
CAAACTACTCAAGAGGAAGATGGCTGTAGCTGCCGATTTCCAGAAGAAGAAGAAGG
AGGATGTGAACTGTGATAA
22. Nucleic acid sequence of 137DCR4 (without signal peptide)
(SEQ ID NO: 22)
CAGGTGCAACTACAGCAGTGGGGCGCAGGACTGTTGAAGCCTTCGGAGACCCTG
TCCCTCACCTGCGCTGTCTATGGTGGGTCCTTCAGTGGTTACTACTGGAGCTGGATAC
GCCAGTCCCCAGAGAAGGGGCTGGAGTGGATTGGGGAAATCAATCATGGTGGATACG
TCACCTACAATCCGTCCCTCGAGAGTCGAGTCACCATATCAGTAGACACGTCCAAGA
ACCAGTTCTCCCTGAAGCTGAGCTCTGTGACCGCCGCGGACACGGCTGTATATTACTG
TGCGAGGGACTATGGTCCGGGGAATTATGACTGGTACTTCGATCTCTGGGGCCGTGGC
ACCCTGGTCACTGTCTCCTCAGGTGGAGGCGGTTCAGGCGGAGGTGGCAGCGGCGGT
GGCGGGTCGGAAATTGTGTTGACACAGTCTCCAGCCACCCTGTCTTTGTCTCCAGGGG
AAAGAGCCACCCTCTCCTGCAGGGCCAGTCAGAGTGTTAGCAGCTACTTAGCCTGGT
ACCAACAGAAACCTGGCCAGGCTCCCAGGCTCCTCATCTATGATGCATCCAACAGGG
CCACTGGCATCCCAGCCAGGTTCAGTGGCAGTGGGTCTGGGACAGACTTCACTCTCAC
CATCAGCAGCCTAGAGCCTGAAGATTTTGCAGTTTATTACTGTCAGCAGCGTAGCAAC
TGGCCTCCGGCGCTCACTTTCGGCGGAGGGACCAAGGTGGAGATCAAAGAGTCCAAA
TATGGTCCCCCATGCCCACCATGCCCAATTGAAGTTATGTATCCTCCTCCTTACCTAGA
CAATGAGAAGAGCAATGGAACCATTATCCATGTGAAAGGGAAACACCTTTGTCCAAG
TCCCCTATTTCCCGGACCTTCTAAGCCCTTTTGGGTGCTGGTGGTGGTTGGTGGAGTCC
TGGCTTGCTATAGCTTGCTAGTAACAGTGGCCTTTATTATTTTCTGGGTGAAACGGGG
CAGAAAGAAGCTCCTGTATATATTCAAACAACCATTTATGAGACCAGTACAAACTAC
TCAAGAGGAAGATGGCTGTAGCTGCCGATTTCCAGAAGAAGAAGAAGGAGGATGTG
AACTGAGGAGTAAGAGGAGCAGGCTCCTGCACAGTGACTACATGAACATGACTCCCC
GCCGCCCCGGGCCCACCCGCAAGCATTACCAGCCCTATGCCCCACCACGCGACTTCGC
AGCCTATCGCTCCTGATAA
23. Amino acid sequence of meso G1 CAR
(SEQ ID NO: 23)
MALPVTALLLPLALLLHAARPSQVQLQQSGPELEKPGASVKISCKASGYSFTGYTMN
WVKQSHGKSLEWIGLITPYNGASSYNQKFRGKATLTVDKSSSTAYMDLLSLTSEDSAVYF
CARGGYDGRGFDYWGQGTTVTVSSGGGGSGGGGSGGGGSDIELTQSPAIMSASPGEKVT
MTCSASSSVSYMHWYQQKSGTSPKRWIYDTSKLASGVPGRFSGSGSGNSYSLTISSVEAE
DDATYYCQQWSKHPLTYGAGTKLEIKFVPVFLPAKPTTTPAPRPPTPAPTIASQPLSLRPEA
CRPAAGGAVHTRGLDFACDIYIWAPLAGTCGVLLLSLVITLYCNHRVKFSRSADAPAYQQ
GQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
24. Nucleic acid sequence of meso G1 CAR
(SEQ ID NO: 24)
ATGGCCTTACCAGTGACCGCCTTGCTCCTGCCGCTGGCCTTGCTGCTCCACGCCG
CCAGGCCGAGCCAGGTACAACTGCAGCAGTCTGGGCCTGAGCTGGAGAAGCCTGGCG
CTTCAGTGAAGATATCCTGCAAGGCTTCTGGTTACTCATTCACTGGCTACACCATGAA
CTGGGTGAAGCAGAGCCATGGAAAGAGCCTTGAGTGGATTGGACTTATTACTCCTTA
CAATGGTGCTTCTAGCTACAACCAGAAGTTCAGGGGCAAGGCCACATTAACTGTAGA
CAAGTCATCCAGCACAGCCTACATGGACCTCCTCAGTCTGACATCTGAAGACTCTGCA
GTCTATTTCTGTGCAAGGGGGGGTTACGACGGGAGGGGTTTTGACTACTGGGGCCAA
GGGACCACGGTCACCGTCTCCTCAGGTGGAGGCGGTTCAGGCGGAGGTGGCAGCGGC
GGTGGCGGGTCGGACATCGAGCTCACTCAGTCTCCAGCAATCATGTCTGCATCTCCAG
GGGAGAAGGTCACCATGACCTGCAGTGCCAGCTCAAGTGTAAGTTACATGCACTGGT
ACCAGCAGAAGTCAGGCACCTCCCCCAAAAGATGGATTTATGACACATCCAAACTGG
CTTCTGGAGTCCCAGGTCGCTTCAGTGGCAGTGGGTCTGGAAACTCTTACTCTCTCAC
AATCAGCAGCGTGGAGGCTGAAGATGATGCAACTTATTACTGCCAGCAGTGGAGTAA
GCACCCTCTCACGTACGGTGCTGGGACAAAGTTGGAAATCAAATTCGTGCCGGTCTTC
CTGCCAGCGAAGCCCACCACGACGCCAGCGCCGCGACCACCAACACCGGCGCCCACC
ATCGCGTCGCAGCCCCTGTCCCTGCGCCCAGAGGCGTGCCGGCCAGCGGCGGGGGGC
GCAGTGCACACGAGGGGGCTGGACTTCGCCTGTGATATCTACATCTGGGCGCCCCTG
GCCGGGACTTGTGGGGTCCTTCTCCTGTCACTGGTTATCACCCTTTACTGCAACCACA
GAGTGAAGTTCAGCAGGAGCGCAGACGCCCCCGCGTACCAGCAGGGCCAGAACCAG
CTCTATAACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGGAT
GAAAGGCGAGCGCCGGAGGGGCAAGGGGCACGATGGCCTTTACCAGGGTCTCAGTA
CAGCCACCAAGGACACCTACGACGCCCTTCACATGCAGGCCCTGCCCCCTCGCTGA
TAA
25. Tyrosine activation motif of CD3ξ
(SEQ ID NO: 25)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
26. Nucleotide sequence of meso G2
CAR (SEQ ID NO: 26)
ATGGCCTTACCAGTGACCGCCTTGCTCCTGCCGCTGGCCTTGCTGCTCCACGCCG
CCAGGCCGAGCGAGGTGCAGCTGGTGGAGTCCGGGGGAGGCCTGGTCCAGCCTGGGG
GATCCCTGAGACTCTCCTGCGCAGCCTCTGGATTCGACCTCGGTTTCTACTTTTACGCC
TGTTGGGTCCGCCAGGCTCCAGGGAAGGGCCTGGAGTGGGTCTCATGCATTTATACTG
CTGGTAGTGGTAGCACGTACTACGCGAGCTGGGCGAAAGGCCGATTCACCATCTCCA
GAGACAATTCGAAGAACACGCTGTATCTGCAAATGAACAGTCTGAGAGCCGAGGACA
CGGCCGTGTATTACTGTGCGAGATCTACTGCTAATACTAGAAGTACTTATTATCTTAA
CTTGTGGGGCCAAGGCACCCTGGTCACCGTCTCCTCAGGCGGAGGCGGATCAGGTGG
TGGCGGATCTGGAGGTGGCGGAAGCGACATCCAGATGACCCAGTCTCCATCCTCCCT
GTCTGCATCTGTGGGAGACAGAGTCACCATCACTTGCCAGGCCAGTCAGAGGATTAG
TAGTTACTTATCCTGGTATCAGCAGAAACCAGGGAAAGTTCCCAAGCTCCTGATCTAT
GGTGCATCCACTCTGGCATCTGGGGTCCCCTCGCGGTTCAGTGGCAGTGGATCTGGGA
CAGATTTCACTCTCACCATCAGCAGCCTGCAGCCTGAAGATGTTGCCACTTACTACTG
TCAGAGTTATGCTTATTTTGATAGTAATAATTGGCATGCTTTCGGCGGAGGGACCAAG
GTGGAGATCAAAGAGTCCAAATATGGTCCCCCATGCCCACCATGCCCAGCACCTCCC
GTGGCCGGACCATCAGTCTTCCTGTTCCCCCCAAAACCCAAGGACACTCTCATGATCT
CCCGGACCCCTGAGGTCACGTGCGTGGTGGTGGACGTGAGCCAGGAAGACCCCGAGG
TCCAGTTCAACTGGTACGTGGATGGCGTGGAGGTGCATAATGCCAAGACAAAGCCGC
GGGAGGAGCAGTTCCAGAGCACGTACCGTGTGGTCAGCGTCCTCACCGTCCTGCACC
AGGACTGGCTGAACGGCAAGGAGTACAAGTGCAAGGTCTCCAACAAAGGCCTCCCGT
CCTCCATCGAGAAAACCATCTCCAAAGCCAAAGGGCAGCCCCGAGAGCCACAGGTGT
ACACCCTGCCCCCATCCCAGGAGGAGATGACCAAGAACCAGGTCAGCCTGACCTGCC
TGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTAACCGTGGACAAGAGCAGGTGGCAGGAGGGGAATGTCTTCTCATG
CTCCGTGATGCATGAGGCTCTGCACAACCACTACACACAGAAGAGCCTCTCCCTGTCT
CTGGGTAAACCCTTTTGGGTGCTGGTGGTGGTTGGTGGAGTCCTGGCTTGCTATAGCT
TGCTAGTAACAGTGGCCTTTATTATTTTCTGGGTGAGGAGTAAGAGGAGCAGGCTCCT
GCACAGTGACTACATGAACATGACTCCCCGCCGCCCCGGGCCCACCCGCAAGCATTA
CCAGCCCTATGCCCCACCACGCGACTTCGCAGCCTATCGCTCCAGAGTGAAGTTCAGC
AGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCT
GAGATGGGGGGAAAGCCGAGAAGGAAGAACCCTCAGGAAGGCCTGTACAATGAACT
GCAGAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGGACA
CCTACGACGCCCTTCACATGCAGGCCCTGCCCCCTCGC
27. Nucleic acid sequence of PS328b vector
(SEQ ID NO: 27)
GGCGCGCCTTAACCCTAGAAAGATAATCATATTGTGACGTACGTTAAAGATAATC
ATGCGTAAAATTGACGCATGTCTAGAGGTGTGGAAAGTCCCCAGGCTCCCCAGCAGG
CAGAAGTATGCAAAGCATGCATCTCAATTAGTCAGCAACCAGGTGTGGAAAGTCCCC
AGGCTCCCCAGCAGGCAGAAGTATGCAAAGCATGCATCTCAATTAGTCAGCAACCAA
GGATCTGCGATCGCTCCGGTGCCCGTCAGTGGGCAGAGCGCACATCGCCCACAGTCC
CCGAGAAGTTGGGGGGAGGGGTCGGCAATTGAACGGGTGCCTAGAGAAGGTGGCGC
GGGGTAAACTGGGAAAGTGATGTCGTGTACTGGCTCCGCCTTTTTCCCGAGGGTGGG
GGAGAACCGTATATAAGTGCAGTAGTCGCCGTGAACGTTCTTTTTCGCAACGGGTTTG
CCGCCAGAACACAGCTGAAGCTTCGAGGGGCTCGCATCTCTCCTTCACGCGCCCGCCG
CCCTACCTGAGGCCGCCATCCACGCCGGTTGAGTCGCGTTCTGCCGCCTCCCGCCTGT
GGTGCCTCCTGAACTGCGTCCGCCGTCTAGGTAAGTTTAAAGCTCAGGTCGAGACCGG
GCCTTTGTCCGGCGCTCCCTTGGAGCCTACCTAGACTCAGCCGGCTCTCCACGCTTTG
CCTGACCCTGCTTGCTCAACTCTACGTCTTTGTTTCGTTTTCTGTTCTGCGCCGTTACA
GATCCAAGCTGTGACCGGCGCCTACGAATTCGGATCCTGCACTAGTGCTGTCGACCAG
ACATGATAAGATACATTGATGAGTTTGGACAAACCACAACTAGAATGCAGTGAAAAA
AATGCTTTATTTGTGAAATTTGTGATGCTATTGCTTTATTTGTAACCATTATAAGCTGC
AATAAACAAGTTAACAACAACAATTGCATTCATTTTATGTTTCAGGTTCAGGGGGAGG
TGTGGGAGGTTTTTTAAAGCAAGTAAAACCTCTACAAATGTGGTAGCATGCGTCAATT
TTACGCAGACTATCTTTCTAGGGTTAAATCGATCCCGCTAGCCCCGATATCCCCTTAA
TTAAGAGGGGGAGACCAAAGGGCGAGACGTTAAGGCCTCACGTGACATGTGAGCAA
AAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCTC
TCCTGTTCCGACCCTGCCGCTTACGGGATACCTGTCCGCCTTTCTCCCTTCGGGAAGCG
TGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCAC
TGGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCAC
GTTAAGGGATTTTGGTCATGCCGTCTCAGAAGAACTCGTCAAGAAGGCGATAGAAGG
CGATGCGCTGCGAATCGGGAGCGGCGATACCGTAAAGCACGAGGAAGCGGTCAGCC
CATTCGCCGCCAAGCTCTTCAGCAATATCACGGGTAGCCAACGCTATGTCCTGATAGC
GGTCCGCCACACCCAGCCGGCCACAGTCGATGAATCCAGAAAAGCGGCCATTTTCCA
CCATGATATTCGGCAAGCAGGCATCGCCATGGGTCACGACGAGATCCTCGCCGTCGG
GCATGCTCGCCTTGAGCCTGGCGAACAGTTCGGCTGGCGCGAGCCCCTGATGCTCTTC
GTCCAGATCATCCTGATCGACAAGACCGGCTTCCATCCGAGTACGTGCTCTCTCGATG
CGATGTTTCGCTTGGTGGTCGAATGGGCAGGTAGCCGGATCAAGCGTATGCAGCCGC
CGCATTGCATCAGCCATGATGGATACTTTCTCGGCAGGAGCAAGGTGAGATGACAGG
AGATCCTGCCCCGGCACTTCGCCCAATAGCAGCCAGTCCCTTCCCGCTTCAGTGACAA
CGTCGAGTACAGCTGCGCAAGGAACGCCCGTCGTGGCCAGCCACGATAGCCGCGCTG
CCTCGTCTTGCAGTTCATTCAGGGCACCGGACAGGTCGGTCTTGACAAAAAGAACCG
GGCGCCCCTGCGCTGACAGCCGGAACACGGCGGCATCAGAGCAGCCGATTGTCTGTT
GTGCCCAGTCATAGCCGAATAGCCTCTCCACCCAAGCGGCCGGAGAACCTGCGTGCA
ATCCATCTTGTTCAATCATAATATTATTGAAGCATTTATCAGGGTTCGTCTCGTCCCGG
TCTCCTCCCATGCATG
28. Nucleotide sequence of primer CD137-F
(SEQ ID NO: 28)
CGAGTCACCATATCAGTA
29. Nucleotide sequence of primer CDI37-R
(SEQ ID NO: 29)
CGAAGTACCAGTCATAATTC
30. Nucleotide sequence in probe Taqman
(SEQ ID NO: 30)
CCTCGCACAGTAATATACAGCCGT
31. Amino acid sequence of CD28 extracellular agonistic
single-chain antibody
(SEQ ID NO: 31)
QVQLVQSGAEVKKPGASVKVSCKASGYTFTSYYIHWVRQAPGQGLEWIGCIYPGNV
NTNYNEKFKDRATLTVDTSISTAYMELSRLRSDDTAVYFCTRSHYGLDWNFDVWGQGTT
VTVSSGGGGSGGGGSGGGGSDIQMTQSPSSLSASVGDRVTITCHASQNIYVWLNWYQQK
PGKAPKLLIYKASNLHTGVPSRFSGSGSGTDFTLTISSLQPEDFATYYCQQGQTYPYTFGG
GTKVEIKE
32. Amino acid sequence of 28DCRI (including signal peptide)
(SEQ ID NO: 32)
MGNSCYNIVATLLLVLNFERTRSQVQLVQSGAEVKKPGASVKVSCKASGYTFTSYYI
HWVRQAPGQGLEWIGCIYPGNVNTNYNEKFKDRATLTVDTSISTAYMELSRLRSDDTAV
YFCTRSHYGLDWNFDVWGQGTTVTVSSGGGGSGGGGSGGGGSDIQMTQSPSSLSASVG
DRVTITCHASQNIYVWLNWYQQKPGKAPKLLIYKASNLHTGVPSRFSGSGSGTDFTLTISS
LQPEDFATYYCQQGQTYPYTFGGGTKVEIRESKYGPPCPPCPIEVMYPPPYLDNEKSNGTII
HVKGKHLCPSPLFPGPSKPFWVLVVVGGVLACYSLLVTVAFIIFWVRSKRSRLLHSDYMN
MTPRRPGPTRKHYQPYAPPRDFAAYRS
33. Amino acid sequence of 28DCR2 (including signal peptide)
(SEQ ID NO: 33)
MGNSCYNIVATLLLVLNFERTRSQVQLVQSGAEVKKPGASVKVSCKASGYTFTSYYI
HWVRQAPGQGLEWIGCIYPGNVNTNYNEKFKDRATLTVDTSISTAYMELSRLRSDDTAV
YFCTRSHYGLDWNFDVWGQGTTVTVSSGGGGSGGGGSGGGGSDIQMTQSPSSLSASVG
DRVTITCHASQNIYVWLNWYQQKPGKAPKLLIYKASNLHTGVPSRFSGSGSGTDFTLTISS
LQPEDFATYYCQQGQTYPYTFGGGTKVEIKESKYGPPCPPCPIEVMYPPPYLDNEKSNGTII
HVKGKHLCPSPLFPGPSKPFWVLVVVGGVLACYSLLVTVAFIIFWVKRGRKKLLYIFKQP
FMRPVQTTQEEDGCSCRFPEEEEGGCEL
34. Amino acid sequence of 28DCR3 (including signal peptide)
(SEQ ID NO: 34)
MGNSCYNIVATLLLVLNFERTRSQVQLVQSGAEVKKPGASVKVSCKASGYTFTSYYI
HWVRQAPGQGLEWIGCIYPGNVNTNYNEKFKDRATLTVDTSISTAYMELSRLRSDDTAV
YFCTRSHYGLDWNFDVWGQGTTVTVSSGGGGSGGGGSGGGGSDIQMTQSPSSLSASVG
DRVTITCHASQNIYVWLNWYQQKPGKAPKLLIYKASNLHTGVPSRFSGSGSGTDFTLTISS
LQPEDFATYYCQQGQTYPYTFGGGTKVEIKESKYGPPCPPCPIEVMYPPPYLDNEKSNGTII
HVKGKHLCPSPLFPGPSKPFWVLVVVGGVLACYSLLVTVAFIIFWVRSKRSRLLHSDYMN
MTPRRPGPTRKHYQPYAPPRDFAAYRSKRGRKKLLYIFKQPFMRPVQTTQEEDGCSCRFP
EEEEGGCEL
35. Amino acid sequence of 28DCR4 (including signal peptide)
(SEQ ID NO: 35)
MGNSCYNIVATLLLVLNFERTRSQVQLVQSGAEVKKPGASVKVSCKASGYTFTSYYI
HWVRQAPGQGLEWIGCIYPGNVNTNYNEKFKDRATLTVDTSISTAYMELSRLRSDDTAV
YFCTRSHYGLDWNFDVWGQGTTVTVSSGGGGSGGGGSGGGGSDIQMTQSPSSLSASVG
DRVTITCHASQNIYVWLNWYQQKPGKAPKLLIYKASNLHTGVPSRFSGSGSGTDFTLTISS
LQPEDFATYYCQQGQTYPYTFGGGTKVEIKESKYGPPCPPCPIEVMYPPPYLDNEKSNGTII
HVKGKHLCPSPLFPGPSKPFWVLVVVGGVLACYSLLVTVAFIIFWVKRGRKKLLYIFKQP
FMRPVQTTQEEDGCSCRFPEEEEGGCELRSKRSRLLHSDYMNMTPRRPGPTRKHYQPYAP
PRDFAAYRS
36. Amino acid sequence of 28DCR1 (without signal peptide)
(SEQ ID NO: 36)
QVQLVQSGAEVKKPGASVKVSCKASGYTFTSYYIHWVRQAPGQGLEWIGCIYPGNV
NTNYNEKFKDRATLTVDTSISTAYMELSRLRSDDTAVYFCTRSHYGLDWNFDVWGQGTT
VTVSSGGGGSGGGGSGGGGSDIQMTQSPSSLSASVGDRVTITCHASQNIYVWLNWYQQK
PGKAPKLLIYKASNLHTGVPSRFSGSGSGTDFTLTISSLQPEDFATYYCQQGQTYPYTFGG
GTKVEIKESKYGPPCPPCPIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDF
AAYRS
37. Amino acid sequence of 28DCR2 (without signal peptide)
(SEQ ID NO: 37)
QVQLVQSGAEVKKPGASVKVSCKASGYTFTSYYIHWVRQAPGQGLEWIGCIYPGNV
NTNYNEKFKDRATLTVDTSISTAYMELSRLRSDDTAVYFCTRSHYGLDWNFDVWGQGTT
VTVSSGGGGSGGGGSGGGGSDIQMTQSPSSLSASVGDRVTITCHASQNIYVWLNWYQQK
PGKAPKLLIYKASNLHTGVPSRFSGSGSGTDFTLTISSLQPEDFATYYCQQGQTYPYTFGG
GTKVEIKESKYGPPCPPCPIEVMYPPPYLDNEKSNGTIIHVKGKHLCPSPLFPGPSKPFWVL
VVVGGVLACYSLLVTVAFIIFWVKRGRKKLLYIFKQPFMRPVQTTQEEDGCSCRFPEEEE
GGCEL
38. Amino acid sequence of 28DCR3 (without signal peptide)
(SEQ ID NO: 38)
QVQLVQSGAEVKKPGASVKVSCKASGYTFTSYYIHWVRQAPGQGLEWIGCIYPGNV
NTNYNEKFKDRATLTVDTSISTAYMELSRLRSDDTAVYFCTRSHYGLDWNFDVWGQGTT
VTVSSGGGGSGGGGSGGGGSDIQMTQSPSSLSASVGDRVTITCHASQNIYVWLNWYQQK
PGKAPKLLIYKASNLHTGVPSRFSGSGSGTDFTLTISSLQPEDFATYYCQQGQTYPYTFGG
GTKVEIKESKYGPPCPPCPIEVMYPPPYLDNEKSNGTIIHVKGKHLCPSPLFPGPSKPFWVL
VVVGGVLACYSLLVTVAFIIFWVRSKRSRLLHSDYMNMTPRRPGPTRKHYQPYAPPRDF
AAYRSKRGRKKLLYIFKQPFMRPVQTTQEEDGCSCRFPEEEEGGCEL
39. Amino acid sequence of 28DCR4 (without signal peptide)
(SEQ ID NO: 39)
QVQLVQSGAEVKKPGASVKVSCKASGYTFTSYYIHWVRQAPGQGLEWIGCIYPGNV
NTNYNEKFKDRATLTVDTSISTAYMELSRLRSDDTAVYFCTRSHYGLDWNFDVWGQGTT
VTVSSGGGGSGGGGSGGGGSDIQMTQSPSSLSASVGDRVTITCHASQNIYVWLNWYQQK
PGKAPKLLIYKASNLHTGVPSRFSGSGSGTDFTLTISSLQPEDFATYYCQQGQTYPYTFGG
GTKVEIKESKYGPPCPPCPIEVMYPPPYLDNEKSNGTIIHVKGKHLCPSPLFPGPSKPFWVL
VVVGGVLACYSLLVTVAFIIFWVKRGRKKLLYIFKQPFMRPVQTTQEEDGCSCRFPEEEE
GGCELRSKRSRLLHSDYMNMTPRRPGPTRKHYQPYAPPRDFAAYRS
40. Nucleic acid sequence of 28DCR1 (including signal peptide)
(SEQ ID NO: 40)
ATGGGAAACAGCTGTTACAACATAGTAGCCACTCTGTTGCTGGTCCTCAACTTTG
AGAGGACAAGATCACAGGTGCAGCTGGTGCAGTCTGGGGCTGAGGTGAAGAAGCCT
GGGGCCTCAGTGAAGGTCTCCTGCAAGGCTTCTGGATACACCTTCACCAGCTACTATA
TACACTGGGTGCGACAGGCCCCTGGACAAGGGCTTGAGTGGATTGGATGTATTTATCC
TGGAAATGTCAATACTAACTATAATGAGAAGTTCAAGGACAGGGCCACCCTGACCGT
AGACACGTCCATCAGCACAGCCTACATGGAGCTGAGCAGGCTGAGATCTGACGACAC
GGCCGTGTATTTCTGTACAAGATCACACTACGGCCTCGACTGGAACTTCGATGTCTGG
GGCCAAGGGACCACGGTCACCGTCTCCTCAGGTGGAGGCGGTTCAGGCGGAGGTGGC
AGCGGCGGTGGCGGGTCGGACATCCAGATGACCCAGTCTCCATCCTCCCTGTCTGCAT
CTGTAGGAGACAGAGTCACCATCACTTGCCATGCCAGTCAAAACATTTATGTTTGGTT
AAACTGGTATCAGCAGAAACCAGGGAAAGCCCCTAAGCTCCTGATCTATAAGGCTTC
CAACCTGCACACAGGGGTCCCATCAAGGTTCAGTGGCAGTGGATCTGGGACAGATTT
CACTCTCACCATCAGCAGTCTGCAACCTGAAGATTTTGCAACTTACTACTGTCAACAG
GGTCAAACTTATCCGTACACGTTCGGCGGAGGGACCAAGGTGGAGATCAAAGAGTCC
AAATATGGTCCCCCATGCCCACCATGCCCAATTGAAGTTATGTATCCTCCTCCTTACCT
AGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGA
GTAAGAGGAGCAGGCTCCTGCACAGTGACTACATGAACATGACTCCCCGCCGCCCCG
GGCCCACCCGCAAGCATTACCAGCCCTATGCCCCACCACGCGACTTCGCAGCCTATCG
CTCCTGATAA
41. Nucleic acid sequence of 28DCR2 (including signal peptide)
(SEQ ID NO: 41)
ATGGGAAACAGCTGTTACAACATAGTAGCCACTCTGTTGCTGGTCCTCAACTTTG
AGAGGACAAGATCACAGGTGCAGCTGGTGCAGTCTGGGGCTGAGGTGAAGAAGCCT
GGGGCCTCAGTGAAGGTCTCCTGCAAGGCTTCTGGATACACCTTCACCAGCTACTATA
TACACTGGGTGCGACAGGCCCCTGGACAAGGGCTTGAGTGGATTGGATGTATTTATCC
TGGAAATGTCAATACTAACTATAATGAGAAGTTCAAGGACAGGGCCACCCTGACCGT
AGACACGTCCATCAGCACAGCCTACATGGAGCTGAGCAGGCTGAGATCTGACGACAC
GGCCGTGTATTTCTGTACAAGATCACACTACGGCCTCGACTGGAACTTCGATGTCTGG
GGCCAAGGGACCACGGTCACCGTCTCCTCAGGTGGAGGCGGTTCAGGCGGAGGTGGC
AGCGGCGGTGGCGGGTCGGACATCCAGATGACCCAGTCTCCATCCTCCCTGTCTGCAT
CTGTAGGAGACAGAGTCACCATCACTTGCCATGCCAGTCAAAACATTTATGTTTGGTT
AAACTGGTATCAGCAGAAACCAGGGAAAGCCCCTAAGCTCCTGATCTATAAGGCTTC
CAACCTGCACACAGGGGTCCCATCAAGGTTCAGTGGCAGTGGATCTGGGACAGATTT
CACTCTCACCATCAGCAGTCTGCAACCTGAAGATTTTGCAACTTACTACTGTCAACAG
GGTCAAACTTATCCGTACACGTTCGGCGGAGGGACCAAGGTGGAGATCAAAGAGTCC
AAATATGGTCCCCCATGCCCACCATGCCCAATTGAAGTTATGTATCCTCCTCCTTACCT
AGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAAACG
GGGCAGAAAGAAGCTCCTGTATATATTCAAACAACCATTTATGAGACCAGTACAAAC
TACTCAAGAGGAAGATGGCTGTAGCTGCCGATTTCCAGAAGAAGAAGAAGGAGGAT
GTGAACTGTGATAA
42. Nucleic acid sequence of 28DCR3 (including signal peptide)
(SEQ ID NO: 42)
ATGGGAAACAGCTGTTACAACATAGTAGCCACTCTGTTGCTGGTCCTCAACTTTG
AGAGGACAAGATCACAGGTGCAGCTGGTGCAGTCTGGGGCTGAGGTGAAGAAGCCT
GGGGCCTCAGTGAAGGTCTCCTGCAAGGCTTCTGGATACACCTTCACCAGCTACTATA
TACACTGGGTGCGACAGGCCCCTGGACAAGGGCTTGAGTGGATTGGATGTATTTATCC
TGGAAATGTCAATACTAACTATAATGAGAAGTTCAAGGACAGGGCCACCCTGACCGT
AGACACGTCCATCAGCACAGCCTACATGGAGCTGAGCAGGCTGAGATCTGACGACAC
GGCCGTGTATTTCTGTACAAGATCACACTACGGCCTCGACTGGAACTTCGATGTCTGG
GGCCAAGGGACCACGGTCACCGTCTCCTCAGGTGGAGGCGGTTCAGGCGGAGGTGGC
AGCGGCGGTGGCGGGTCGGACATCCAGATGACCCAGTCTCCATCCTCCCTGTCTGCAT
CTGTAGGAGACAGAGTCACCATCACTTGCCATGCCAGTCAAAACATTTATGTTTGGTT
AAACTGGTATCAGCAGAAACCAGGGAAAGCCCCTAAGCTCCTGATCTATAAGGCTTC
CAACCTGCACACAGGGGTCCCATCAAGGTTCAGTGGCAGTGGATCTGGGACAGATTT
CACTCTCACCATCAGCAGTCTGCAACCTGAAGATTTTGCAACTTACTACTGTCAACAG
GGTCAAACTTATCCGTACACGTTCGGCGGAGGGACCAAGGTGGAGATCAAAGAGTCC
AAATATGGTCCCCCATGCCCACCATGCCCAATTGAAGTTATGTATCCTCCTCCTTACCT
AGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGA
GTAAGAGGAGCAGGCTCCTGCACAGTGACTACATGAACATGACTCCCCGCCGCCCCG
GGCCCACCCGCAAGCATTACCAGCCCTATGCCCCACCACGCGACTTCGCAGCCTATCG
CTCCAAACGGGGCAGAAAGAAGCTCCTGTATATATTCAAACAACCATTTATGAGACC
AGTACAAACTACTCAAGAGGAAGATGGCTGTAGCTGCCGATTTCCAGAAGAAGAAGA
AGGAGGATGTGAACTGTGATAA
43. Nucleic acid sequence of 28DCR4 (including signal peptide)
(SEQ ID NO: 43)
ATGGGAAACAGCTGTTACAACATAGTAGCCACTCTGTTGCTGGTCCTCAACTTTG
AGAGGACAAGATCACAGGTGCAGCTGGTGCAGTCTGGGGCTGAGGTGAAGAAGCCT
GGGGCCTCAGTGAAGGTCTCCTGCAAGGCTTCTGGATACACCTTCACCAGCTACTATA
TACACTGGGTGCGACAGGCCCCTGGACAAGGGCTTGAGTGGATTGGATGTATTTATCC
TGGAAATGTCAATACTAACTATAATGAGAAGTTCAAGGACAGGGCCACCCTGACCGT
AGACACGTCCATCAGCACAGCCTACATGGAGCTGAGCAGGCTGAGATCTGACGACAC
GGCCGTGTATTTCTGTACAAGATCACACTACGGCCTCGACTGGAACTTCGATGTCTGG
GGCCAAGGGACCACGGTCACCGTCTCCTCAGGTGGAGGCGGTTCAGGCGGAGGTGGC
AGCGGCGGTGGCGGGTCGGACATCCAGATGACCCAGTCTCCATCCTCCCTGTCTGCAT
CTGTAGGAGACAGAGTCACCATCACTTGCCATGCCAGTCAAAACATTTATGTTTGGTT
AAACTGGTATCAGCAGAAACCAGGGAAAGCCCCTAAGCTCCTGATCTATAAGGCTTC
CAACCTGCACACAGGGGTCCCATCAAGGTTCAGTGGCAGTGGATCTGGGACAGATTT
CACTCTCACCATCAGCAGTCTGCAACCTGAAGATTTTGCAACTTACTACTGTCAACAG
GGTCAAACTTATCCGTACACGTTCGGCGGAGGGACCAAGGTGGAGATCAAAGAGTCC
AAATATGGTCCCCCATGCCCACCATGCCCAATTGAAGTTATGTATCCTCCTCCTTACCT
AGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAAACG
GGGCAGAAAGAAGCTCCTGTATATATTCAAACAACCATTTATGAGACCAGTACAAAC
TACTCAAGAGGAAGATGGCTGTAGCTGCCGATTTCCAGAAGAAGAAGAAGGAGGAT
GTGAACTGAGGAGTAAGAGGAGCAGGCTCCTGCACAGTGACTACATGAACATGACTC
CCCGCCGCCCCGGGCCCACCCGCAAGCATTACCAGCCCTATGCCCCACCACGCGACTT
CGCAGCCTATCGCTCCTGATAA
44. Nucleic acid sequence of 28DCR1 (without signal peptide)
(SEQ ID NO: 44)
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGCTACTATATACACTGGGTGC
GACAGGCCCCTGGACAAGGGCTTGAGTGGATTGGATGTATTTATCCTGGAAATGTCA
ATACTAACTATAATGAGAAGTTCAAGGACAGGGCCACCCTGACCGTAGACACGTCCA
TCAGCACAGCCTACATGGAGCTGAGCAGGCTGAGATCTGACGACACGGCCGTGTATT
TCTGTACAAGATCACACTACGGCCTCGACTGGAACTTCGATGTCTGGGGCCAAGGGA
CCACGGTCACCGTCTCCTCAGGTGGAGGCGGTTCAGGCGGAGGTGGCAGCGGCGGTG
GCGGGTCGGACATCCAGATGACCCAGTCTCCATCCTCCCTGTCTGCATCTGTAGGAGA
CAGAGTCACCATCACTTGCCATGCCAGTCAAAACATTTATGTTTGGTTAAACTGGTAT
CAGCAGAAACCAGGGAAAGCCCCTAAGCTCCTGATCTATAAGGCTTCCAACCTGCAC
ACAGGGGTCCCATCAAGGTTCAGTGGCAGTGGATCTGGGACAGATTTCACTCTCACC
ATCAGCAGTCTGCAACCTGAAGATTTTGCAACTTACTACTGTCAACAGGGTCAAACTT
ATCCGTACACGTTCGGCGGAGGGACCAAGGTGGAGATCAAAGAGTCCAAATATGGTC
CCCCATGCCCACCATGCCCAATTGAAGTTATGTATCCTCCTCCTTACCTAGACAATGA
GAAGAGCAATGGAACCATTATCCATGTGAAAGGGAAACACCTTTGTCCAAGTCCCCT
ATTTCCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAGGAGTAAGAGGAG
CAGGCTCCTGCACAGTGACTACATGAACATGACTCCCCGCCGCCCCGGGCCCACCCG
CAAGCATTACCAGCCCTATGCCCCACCACGCGACTTCGCAGCCTATCGCTCCTGATAA
45. Nucleic acid sequence of 28DCR2 (without signal peptide)
(SEQ ID NO: 45)
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGCTACTATATACACTGGGTGC
GACAGGCCCCTGGACAAGGGCTTGAGTGGATTGGATGTATTTATCCTGGAAATGTCA
ATACTAACTATAATGAGAAGTTCAAGGACAGGGCCACCCTGACCGTAGACACGTCCA
TCAGCACAGCCTACATGGAGCTGAGCAGGCTGAGATCTGACGACACGGCCGTGTATT
TCTGTACAAGATCACACTACGGCCTCGACTGGAACTTCGATGTCTGGGGCCAAGGGA
CCACGGTCACCGTCTCCTCAGGTGGAGGCGGTTCAGGCGGAGGTGGCAGCGGCGGTG
GCGGGTCGGACATCCAGATGACCCAGTCTCCATCCTCCCTGTCTGCATCTGTAGGAGA
CAGAGTCACCATCACTTGCCATGCCAGTCAAAACATTTATGTTTGGTTAAACTGGTAT
CAGCAGAAACCAGGGAAAGCCCCTAAGCTCCTGATCTATAAGGCTTCCAACCTGCAC
ACAGGGGTCCCATCAAGGTTCAGTGGCAGTGGATCTGGGACAGATTTCACTCTCACC
ATCAGCAGTCTGCAACCTGAAGATTTTGCAACTTACTACTGTCAACAGGGTCAAACTT
ATCCGTACACGTTCGGCGGAGGGACCAAGGTGGAGATCAAAGAGTCCAAATATGGTC
CCCCATGCCCACCATGCCCAATTGAAGTTATGTATCCTCCTCCTTACCTAGACAATGA
GAAGAGCAATGGAACCATTATCCATGTGAAAGGGAAACACCTTTGTCCAAGTCCCCT
ATTTCCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAAACGGGGCAGAAA
GAAGCTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGTG
ATAA
46. Nucleic acid sequence of 28DCR3 (without signal peptide)
(SEQ ID NO: 46)
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGCTACTATATACACTGGGTGC
GACAGGCCCCTGGACAAGGGCTTGAGTGGATTGGATGTATTTATCCTGGAAATGTCA
ATACTAACTATAATGAGAAGTTCAAGGACAGGGCCACCCTGACCGTAGACACGTCCA
TCAGCACAGCCTACATGGAGCTGAGCAGGCTGAGATCTGACGACACGGCCGTGTATT
TCTGTACAAGATCACACTACGGCCTCGACTGGAACTTCGATGTCTGGGGCCAAGGGA
CCACGGTCACCGTCTCCTCAGGTGGAGGCGGTTCAGGCGGAGGTGGCAGCGGCGGTG
GCGGGTCGGACATCCAGATGACCCAGTCTCCATCCTCCCTGTCTGCATCTGTAGGAGA
CAGAGTCACCATCACTTGCCATGCCAGTCAAAACATTTATGTTTGGTTAAACTGGTAT
CAGCAGAAACCAGGGAAAGCCCCTAAGCTCCTGATCTATAAGGCTTCCAACCTGCAC
ACAGGGGTCCCATCAAGGTTCAGTGGCAGTGGATCTGGGACAGATTTCACTCTCACC
ATCAGCAGTCTGCAACCTGAAGATTTTGCAACTTACTACTGTCAACAGGGTCAAACTT
ATCCGTACACGTTCGGCGGAGGGACCAAGGTGGAGATCAAAGAGTCCAAATATGGTC
CCCCATGCCCACCATGCCCAATTGAAGTTATGTATCCTCCTCCTTACCTAGACAATGA
GAAGAGCAATGGAACCATTATCCATGTGAAAGGGAAACACCTTTGTCCAAGTCCCCT
ATTTCCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAGGAGTAAGAGGAG
CAGGCTCCTGCACAGTGACTACATGAACATGACTCCCCGCCGCCCCGGGCCCACCCG
CAAGCATTACCAGCCCTATGCCCCACCACGCGACTTCGCAGCCTATCGCTCCAAACGG
GGCAGAAAGAAGCTCCTGTATATATTCAAACAACCATTTATGAGACCAGTACAAACT
ACTCAAGAGGAAGATGGCTGTAGCTGCCGATTTCCAGAAGAAGAAGAAGGAGGATG
TGAACTGTGATAA
47. Nucleic acid sequence of 28DCR4 (without signal peptide)
(SEQ ID NO: 47)
CAGGTGCAGCTGGTGCAGTCTGGGGCTGAGGTGAAGAAGCCTGGGGCCTCAGTG
AAGGTCTCCTGCAAGGCTTCTGGATACACCTTCACCAGCTACTATATACACTGGGTGC
GACAGGCCCCTGGACAAGGGCTTGAGTGGATTGGATGTATTTATCCTGGAAATGTCA
ATACTAACTATAATGAGAAGTTCAAGGACAGGGCCACCCTGACCGTAGACACGTCCA
TCAGCACAGCCTACATGGAGCTGAGCAGGCTGAGATCTGACGACACGGCCGTGTATT
TCTGTACAAGATCACACTACGGCCTCGACTGGAACTTCGATGTCTGGGGCCAAGGGA
CCACGGTCACCGTCTCCTCAGGTGGAGGCGGTTCAGGCGGAGGTGGCAGCGGCGGTG
GCGGGTCGGACATCCAGATGACCCAGTCTCCATCCTCCCTGTCTGCATCTGTAGGAGA
CAGAGTCACCATCACTTGCCATGCCAGTCAAAACATTTATGTTTGGTTAAACTGGTAT
CAGCAGAAACCAGGGAAAGCCCCTAAGCTCCTGATCTATAAGGCTTCCAACCTGCAC
ACAGGGGTCCCATCAAGGTTCAGTGGCAGTGGATCTGGGACAGATTTCACTCTCACC
ATCAGCAGTCTGCAACCTGAAGATTTTGCAACTTACTACTGTCAACAGGGTCAAACTT
ATCCGTACACGTTCGGCGGAGGGACCAAGGTGGAGATCAAAGAGTCCAAATATGGTC
CCCCATGCCCACCATGCCCAATTGAAGTTATGTATCCTCCTCCTTACCTAGACAATGA
GAAGAGCAATGGAACCATTATCCATGTGAAAGGGAAACACCTTTGTCCAAGTCCCCT
ATTTCCCGGACCTTCTAAGCCCTTTTGGGTGCTGGTGGTGGTTGGTGGAGTCCTGGCTT
GCTATAGCTTGCTAGTAACAGTGGCCTTTATTATTTTCTGGGTGAAACGGGGCAGAAA
GAAGCTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGA
GGAGTAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCAGCCTA
TCGCTCCTGATAA
48. Amino acid sequence of Muc1 G1 CAR
(SEQ ID NO: 48)
MALPVTALLLPLALLLHAARPSEVQLQQSGGGLVQPGGSMKLSCVASGFTFSNYWM
NWVRQSPEKGLEWVAEIRLKSNNYATHYAESVKGRFTISRDDSKSSVYLQMNNLRAEDT
GIYYCTFGNSFAYWGQGTTVTVSSGGSGSGGSGSGGSGSDIVVTQESALTTSPGETVTLTC
RSSTGAVTTSNYANWVQEKPDHLFTGLIGGTNNRAPGVPARFSGSLIGDKAALTITGAQT
EDEAIYFCALWYSNHWVFGGGTKLTVLGSEESKYGPPCPPCPAPPVAGPSVFLFPPKPKDT
LMISRTPEVTCVVVDVSQEDPEVQFNWYVDGVEVHNAKTKPREEQFQSTYRVVSVLTVL
HQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVM
HEALHNHYTQKSLSLSLGKFVPVFLPAKPTTTPAPRPPTPAPTIASQPLSLRPEACRPAAGG
AVHTRGLDFACDIYIWAPLAGTCGVLLLSLVITLYCNHKRGRKKLLYIFKQPFMRPVQTT
QEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTAT
KDTYDALHMQALPPR
49. Nucleic acid sequence of Muc1 G1 CAR
(SEQ ID NO: 49)
CCATGCCCACCATGCCCAGCACCTCCCGTGGCCGGACCATCAGTCTTCCTGTTCC
CCCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCGT
GGAGGTGCATAATGCCAAGACAAAGCCGCGGGAGGAGCAGTTCCAGAGCACGTACC
GTGTGGTCAGCGTCCTCACCGTCCTGCACCAGGACTGGCTGAACGGCAAGGAGTACA
AGTGCAAGGTCTCCAACAAAGGCCTCCCGTCCTCCATCGAGAAAACCATCTCCAAAG
CCAAAGGGCAGCCCCGAGAGCCACAGGTGTACACCCTGCCCCCATCCCAGGAGGAGA
TGACCAAGAACCAGGTCAGCCTGACCTGCCTGGTCAAAGGCTTCTACCCCAGCGACA
TCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTAACCGTGGACAAGA
GCAGGTGGCAGGAGGGGAATGTCTTCTCATGCTCCGTGATGCATGAGGCTCTGCACA
ACCACTACACACAGAAGAGCCTCTCCCTGTCTCTGGGTAAATTCGTGCCGGTCTTCCT
GCCAGCGAAGCCCACCACGACGCCAGCGCCGCGACCACCAACACCGGCGCCCACCAT
CGCGTCGCAGCCCCTGTCCCTGCGCCCAGAGGCGTGCCGGCCAGCGGCGGGGGGCGC
AGTGCACACGAGGGGGCTGGACTTCGCCTGTGATATCTACATCTGGGCGCCCCTGGCC
GGGACTTGTGGGGTCCTTCTCCTGTCACTGGTTATCACCCTTTACTGCAACCACAAAC
GGGGCAGAAAGAAGCTCCTGTATATATTCAAACAACCATTTATGAGACCAGTACAAA
CTACTCAAGAGGAAGATGGCTGTAGCTGCCGATTTCCAGAAGAAGAAGAAGGAGGAT
GTGAACTGAGAGTGAAGTTCAGCAGGAGCGCAGACGCCCCCGCGTACCAGCAGGGCC
AGAACCAGCTCTATAACGAGCTCAATCTAGGACGAAGAGAGGAGTACGATGTTTTGG
ACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGA
GATTGGGATGAAAGGCGAGCGCCGGAGGGGCAAGGGGCACGATGGCCTTTACCAGG
GTCTCAGTACAGCCACCAAGGACACCTACGACGCCCTTCACATGCAGGCCCTGCCCCC
TCGC
50. Nucleotide sequence of Muc1 G2 CAR
(SEQ ID NO: 50)
ATGGCCTTACCAGTGACCGCCTTGCTCCTGCCGCTGGCCTTGCTGCTCCACGCCG
CCAGGCCGAGCGAGGTCCAGCTGCAGCAGTCAGGAGGAGGCTTGGTGCAACCTGGAG
GATCCATGAAACTCTCCTGTGTTGCCTCTGGATTCACTTTCAGTAACTACTGGATGAA
CTGGGTCCGCCAGTCTCCAGAGAAGGGGCTTGAGTGGGTTGCTGAAATTAGATTGAA
ATCTAATAATTATGCAACACATTATGCGGAGTCTGTGAAAGGGAGGTTCACCATCTCA
AGAGATGATTCCAAAAGTAGTGTCTACCTGCAAATGAACAACTTAAGAGCTGAAGAC
ACTGGCATTTATTACTGTACCTTTGGTAACTCCTTTGCTTACTGGGGCCAAGGGACCA
CGGTCACCGTCTCCTCAGGTGGTTCTGGTTCTGGCGGCTCCGGTTCCGGTGGATCCGG
CTCTGATATCGTTGTGACTCAGGAATCTGCACTCACCACATCACCTGGTGAAACAGTC
ACACTCACTTGTCGCTCAAGTACTGGGGCTGTTACAACTAGTAACTATGCCAACTGGG
TCCAAGAAAAACCAGATCATTTATTCACTGGTCTAATAGGTGGTACCAACAACCGAG
CACCAGGTGTTCCTGCCAGATTCTCAGGCTCCCTGATTGGAGACAAGGCTGCCCTCAC
CATCACAGGGGCACAGACTGAGGATGAGGCAATATATTTCTGTGCTCTATGGTACAG
CAACCATTGGGTGTTCGGTGGAGGAACCAAACTGACTGTCCTAGGATCCGAGGAGTC
CAAATATGGTCCCCCATGCCCACCATGCCCAGCACCTCCCGTGGCCGGACCATCAGTC
TTCCTGTTCCCCCCAAAACCCAAGGACACTCTCATGATCTCCCGGACCCCTGAGGTCA
CGTGCGTGGTGGTGGACGTGAGCCAGGAAGACCCCGAGGTCCAGTTCAACTGGTACG
TGGATGGCGTGGAGGTGCATAATGCCAAGACAAAGCCGCGGGAGGAGCAGTTCCAG
AGCACGTACCGTGTGGTCAGCGTCCTCACCGTCCTGCACCAGGACTGGCTGAACGGC
AAGGAGTACAAGTGCAAGGTCTCCAACAAAGGCCTCCCGTCCTCCATCGAGAAAACC
ATCTCCAAAGCCAAAGGGCAGCCCCGAGAGCCACAGGTGTACACCCTGCCCCCATCC
CAGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAA
GACCACGCCTCCCGTGCTGGACTCCGACGGCTCCTTCTTCCTCTACAGCAGGCTAACC
GTGGACAAGAGCAGGTGGCAGGAGGGGAATGTCTTCTCATGCTCCGTGATGCATGAG
GCTCTGCACAACCACTACACACAGAAGAGCCTCTCCCTGTCTCTGGGTAAATTCGTGC
CGGTCTTCCTGCCAGCGAAGCCCACCACGACGCCAGCGCCGCGACCACCAACACCGG
CGCCCACCATCGCGTCGCAGCCCCTGTCCCTGCGCCCAGAGGCGTGCCGGCCAGCGG
CGGGGGGCGCAGTGCACACGAGGGGGCTGGACTTCGCCTGTGATATCTACATCTGGG
CGCCCCTGGCCGGGACTTGTGGGGTCCTTCTCCTGTCACTGGTTATCACCCTTTACTGC
AACCACAAACGGGGCAGAAAGAAGCTCCTGTATATATTCAAACAACCATTTATGAGA
CCAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGAGA
AGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCACATGCA
GGCCCTGCCCCCTCGC
51. Nucleotide sequence of primer CD28-F
(SEQ ID NO: 51)
GCTTCTGGATACACCTTC
52. Nucleotide sequence of primer CD28-R
(SEQ ID NO: 52)
CCTTGAACTTCTCATTATAGTTAG
53. Nucleotide sequence in probe Taqman
(SEQ ID NO: 53)
AATACATCCAATCCACTCAAGCC
54. Amino acid sequence of CD8 signal peptide
(SEQ ID NO: 54)
MALPVTALLLPLALLLHAARPS
55. Amino acid sequence of CD40 extracellular agonistic
single-chain antibody
(SEQ ID NO: 55)
QVQLVQSGAEVKKPGASVKVSCKASGYTFTGYYMHWVRQAPGQGLEWMGWINPD
SGGTNYAQKFQGRVTMTRDTSISTAYMELNRLRSDDTAVYYCARDQPLGYCTNGVCSYF
DYWGQGTLVTVSSGGGGSGGGGSGGGGSDIQMTQSPSSVSASVGDRVTITCRASQGIYS
WLAWYQQKPGKAPNLLIYTASTLQSGVPSRFSGSGSGTDFTLTISSLQPEDFATYYCQQA
NIFPLTFGGGTKVEIK
56.Amino acid sequence of IgG4Fc CH2CH3 hinge region
(SEQ ID NO: 56)
ESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLSLGK
57. Amino acid sequence of 40DCR1 (including signal peptide)
(SEQ ID NO: 57)
MALPVTALLLPLALLLHAARPSQVQLVQSGAEVKKPGASVKVSCKASGYTFTGYYM
HWVRQAPGQGLEWMGWINPDSGGTNYAQKFQGRVTMTRDTSISTAYMELNRLRSDDT
AVYYCARDQPLGYCTNGVCSYFDYWGQGTLVTVSSGGGGSGGGGSGGGGSDIQMTQSP
SSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKESKYGPPCPPCPAPEFLGGPSVFLF
PPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGKPFWVLVVVGGVLACYSLLVTVAFIIFWVRSKRSRL
LHSDYMNMTPRRPGPTRKHYQPYAPPRDFAAYRS
58. Amino acid sequence of 40DCR2 (including signal peptide)
(SEQ ID NO: 58)
MALPVTALLLPLALLLHAARPSQVQLVQSGAEVKKPGASVKVSCKASGYTFTGYYM
HWVRQAPGQGLEWMGWINPDSGGTNYAQKFQGRVTMTRDTSISTAYMELNRLRSDDT
AVYYCARDQPLGYCTNGVCSYFDYWGQGTLVTVSSGGGGSGGGGSGGGGSDIQMTQSP
SSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKESKYGPPCPPCPAPEFLGGPSVFLF
PPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGKPFWVLVVVGGVLACYSLLVTVAFIIFWVKRGRKK
LLYIFKQPFMRPVQTTQEEDGCSCRFPEEEEGGCEL
59. Amino acid sequence of 40DCR3 (including signal peptide)
(SEQ ID NO: 59)
MALPVTALLLPLALLLHAARPSQVQLVQSGAEVKKPGASVKVSCKASGYTFTGYYM
HWVRQAPGQGLEWMGWINPDSGGTNYAQKFQGRVTMTRDTSISTAYMELNRLRSDDT
AVYYCARDQPLGYCTNGVCSYFDYWGQGTLVTVSSGGGGSGGGGSGGGGSDIQMTQSP
SSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKESKYGPPCPPCPAPEFLGGPSVFLF
PPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGKPFWVLVVVGGVLACYSLLVTVAFIIFWVRSKRSRL
LHSDYMNMTPRRPGPTRKHYQPYAPPRDFAAYRSKRGRKKLLYIFKQPFMRPVQTTQEE
DGCSCRFPEEEEGGCEL
60. Amino acid sequence of 40DCR4 (including signal peptide)
(SEQ ID NO: 60)
MALPVTALLLPLALLLHAARPSQVQLVQSGAEVKKPGASVKVSCKASGYTFTGYYM
HWVRQAPGQGLEWMGWINPDSGGTNYAQKFQGRVTMTRDTSISTAYMELNRLRSDDT
AVYYCARDQPLGYCTNGVCSYFDYWGQGTLVTVSSGGGGSGGGGSGGGGSDIQMTQSP
SSVSASVGDRVTITCRASQGIYSWLAWYQQKPGKAPNLLIYTASTLQSGVPSRFSGSGSGT
DFTLTISSLQPEDFATYYCQQANIFPLTFGGGTKVEIKESKYGPPCPPCPAPEFLGGPSVFLF
PPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQ
VSLTCLVKGFYPSDIAVEWESNGQPENNYKTTPPVLDSDGSFFLYSRLTVDKSRWQEGNV
FSCSVMHEALHNHYTQKSLSLSLGKPFWVLVVVGGVLACYSLLVTVAFIIFWVKRGRKK
LLYIFKQPFMRPVQTTQEEDGCSCRFPEEEEGGCELRSKRSRLLHSDYMNMTPRRPGPTR
KHYQPYAPPRDFAAYRS
61. Amino acid sequence of 40DCR1 (without signal peptide)
(SEQ ID NO: 61)
QVQLVQSGAEVKKPGASVKVSCKASGYTFTGYYMHWVRQAPGQGLEWMGWINPD
SGGTNYAQKFQGRVTMTRDTSISTAYMELNRLRSDDTAVYYCARDQPLGYCTNGVCSYF
DYWGQGTLVTVSSGGGGSGGGGSGGGGSDIQMTQSPSSVSASVGDRVTITCRASQGIYS
WLAWYQQKPGKAPNLLIYTASTLQSGVPSRFSGSGSGTDFTLTISSLQPEDFATYYCQQA
NIFPLTFGGGTKVEIKESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLS
LSLGKPFWVLVVVGGVLACYSLLVTVAFIIFWVRSKRSRLLHSDYMNMTPRRPGPTRKH
YQPYAPPRDFAAYRS
62. Amino acid sequence of 40DCR2 (without signal peptide)
(SEQ ID NO: 62)
QVQLVQSGAEVKKPGASVKVSCKASGYTFTGYYMHWVRQAPGQGLEWMGWINPD
SGGTNYAQKFQGRVTMTRDTSISTAYMELNRLRSDDTAVYYCARDQPLGYCTNGVCSYF
DYWGQGTLVTVSSGGGGSGGGGSGGGGSDIQMTQSPSSVSASVGDRVTITCRASQGIYS
WLAWYQQKPGKAPNLLIYTASTLQSGVPSRFSGSGSGTDFTLTISSLQPEDFATYYCQQA
NIFPLTFGGGTKVEIKESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLS
LSLGKPFWVLVVVGGVLACYSLLVTVAFIIFWVKRGRKKLLYIFKQPFMRPVQTTQEEDG
CSCRFPEEEEGGCEL
63. Amino acid sequence of 40DCR3 (without signal peptide)
(SEQ ID NO: 63)
QVQLVQSGAEVKKPGASVKVSCKASGYTFTGYYMHWVRQAPGQGLEWMGWINPD
SGGTNYAQKFQGRVTMTRDTSISTAYMELNRLRSDDTAVYYCARDQPLGYCTNGVCSYF
DYWGQGTLVTVSSGGGGSGGGGSGGGGSDIQMTQSPSSVSASVGDRVTITCRASQGIYS
WLAWYQQKPGKAPNLLIYTASTLQSGVPSRFSGSGSGTDFTLTISSLQPEDFATYYCQQA
NIFPLTFGGGTKVEIKESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLS
LSLGKPFWVLVVVGGVLACYSLLVTVAFIIFWVRSKRSRLLHSDYMNMTPRRPGPTRKH
YQPYAPPRDFAAYRSKRGRKKLLYIFKQPFMRPVQTTQEEDGCSCRFPEEEEGGCEL
64. Amino acid sequence of 40DCR4 (without signal peptide)
(SEQ ID NO: 64)
QVQLVQSGAEVKKPGASVKVSCKASGYTFTGYYMHWVRQAPGQGLEWMGWINPD
SGGTNYAQKFQGRVTMTRDTSISTAYMELNRLRSDDTAVYYCARDQPLGYCTNGVCSYF
DYWGQGTLVTVSSGGGGSGGGGSGGGGSDIQMTQSPSSVSASVGDRVTITCRASQGIYS
WLAWYQQKPGKAPNLLIYTASTLQSGVPSRFSGSGSGTDFTLTISSLQPEDFATYYCQQA
NIFPLTFGGGTKVEIKESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLS
LSLGKPFWVLVVVGGVLACYSLLVTVAFIIFWVKRGRKKLLYIFKQPFMRPVQTTQEEDG
CSCRFPEEEEGGCELRSKRSRLLHSDYMNMTPRRPGPTRKHYQPYAPPRDFAAYRS
65. Nucleic acid sequence of 40DCR1 (including signal peptide)
(SEQ ID NO: 65)
ATGGCCTTACCAGTGACCGCCTTGCTCCTGCCGCTGGCCTTGCTGCTCCACGCCG
CCAGGCCGAGCCAGGTGCAGCTGGTGCAGTCTGGGGCTGAGGTGAAGAAGCCTGGGG
CCTCAGTGAAGGTCTCCTGCAAGGCTTCTGGATACACCTTCACCGGCTACTATATGCA
CTGGGTGCGACAGGCCCCTGGACAAGGGCTTGAGTGGATGGGATGGATCAACCCTGA
CAGTGGTGGCACAAACTATGCACAGAAGTTTCAGGGCAGGGTCACCATGACCAGGGA
CACGTCCATCAGCACAGCCTACATGGAGCTGAACAGGCTGAGATCTGACGACACGGC
CGTGTATTACTGTGCGAGAGATCAGCCCCTAGGATATTGTACTAATGGTGTATGCTCC
TACTTTGACTACTGGGGCCAGGGAACCCTGGTCACCGTCTCCTCAGGTGGAGGCGGTT
CAGGCGGAGGTGGCAGCGGCGGTGGCGGGTCGGACATCCAGATGACCCAGTCTCCAT
CTTCCGTGTCTGCATCTGTAGGAGACAGAGTCACCATCACTTGTCGGGCGAGTCAGGG
TATTTACAGCTGGTTAGCCTGGTATCAGCAGAAACCAGGGAAAGCCCCTAACCTCCTG
ATCTATACTGCATCCACTTTACAAAGTGGGGTCCCATCAAGGTTCAGCGGCAGTGGAT
CTGGGACAGATTTCACTCTCACCATCAGCAGCCTGCAACCTGAAGATTTTGCAACTTA
CTATTGTCAACAGGCTAACATTTTCCCGCTCACTTTCGGCGGAGGGACCAAGGTGGAG
ATCAAAGAGTCCAAATATGGTCCCCCATGCCCACCATGCCCAGCACCTGAGTTCCTGG
GGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGT
CAAAGGCTTCTACCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTAC
AGCAGGCTAACCGTGGACAAGAGCAGGTGGCAGGAGGGGAATGTCTTCTCATGCTCC
GTGATGCATGAGGCTCTGCACAACCACTACACACAGAAGAGCCTCTCCCTGTCTCTGG
GTAAACCCTTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGCTCCTGCAC
AGTGACTACATGAACATGACTCCCCGCCGCCCCGGGCCCACCCGCAAGCATTACCAG
CCCTATGCCCCACCACGCGACTTCGCAGCCTATCGCTCC
66. Nucleic acid sequence of 40DCR2 (including signal peptide)
(SEQ ID NO: 66)
ATGGCCTTACCAGTGACCGCCTTGCTCCTGCCGCTGGCCTTGCTGCTCCACGCCG
CCAGGCCGAGCCAGGTGCAGCTGGTGCAGTCTGGGGCTGAGGTGAAGAAGCCTGGGG
CCTCAGTGAAGGTCTCCTGCAAGGCTTCTGGATACACCTTCACCGGCTACTATATGCA
CTGGGTGCGACAGGCCCCTGGACAAGGGCTTGAGTGGATGGGATGGATCAACCCTGA
CAGTGGTGGCACAAACTATGCACAGAAGTTTCAGGGCAGGGTCACCATGACCAGGGA
CACGTCCATCAGCACAGCCTACATGGAGCTGAACAGGCTGAGATCTGACGACACGGC
CGTGTATTACTGTGCGAGAGATCAGCCCCTAGGATATTGTACTAATGGTGTATGCTCC
TACTTTGACTACTGGGGCCAGGGAACCCTGGTCACCGTCTCCTCAGGTGGAGGCGGTT
CAGGCGGAGGTGGCAGCGGCGGTGGCGGGTCGGACATCCAGATGACCCAGTCTCCAT
CTTCCGTGTCTGCATCTGTAGGAGACAGAGTCACCATCACTTGTCGGGCGAGTCAGGG
TATTTACAGCTGGTTAGCCTGGTATCAGCAGAAACCAGGGAAAGCCCCTAACCTCCTG
ATCTATACTGCATCCACTTTACAAAGTGGGGTCCCATCAAGGTTCAGCGGCAGTGGAT
CTGGGACAGATTTCACTCTCACCATCAGCAGCCTGCAACCTGAAGATTTTGCAACTTA
CTATTGTCAACAGGCTAACATTTTCCCGCTCACTTTCGGCGGAGGGACCAAGGTGGAG
ATCAAAGAGTCCAAATATGGTCCCCCATGCCCACCATGCCCAGCACCTGAGTTCCTGG
GGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGT
CAAAGGCTTCTACCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTAC
AGCAGGCTAACCGTGGACAAGAGCAGGTGGCAGGAGGGGAATGTCTTCTCATGCTCC
GTGATGCATGAGGCTCTGCACAACCACTACACACAGAAGAGCCTCTCCCTGTCTCTGG
GTAAACCCTTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGCTCCTGCAC
AGTGACTACATGAACATGACTCCCCGCCGCCCCGGGCCCACCCGCAAGCATTACCAG
CCCTATGCCCCACCACGCGACTTCGCAGCCTATCGCTCC
67. Nucleic acid sequence of 40DCR3 (including signal peptide)
(SEQ ID NO: 67)
ATGGCCTTACCAGTGACCGCCTTGCTCCTGCCGCTGGCCTTGCTGCTCCACGCCG
CCAGGCCGAGCCAGGTGCAGCTGGTGCAGTCTGGGGCTGAGGTGAAGAAGCCTGGGG
CCTCAGTGAAGGTCTCCTGCAAGGCTTCTGGATACACCTTCACCGGCTACTATATGCA
CTGGGTGCGACAGGCCCCTGGACAAGGGCTTGAGTGGATGGGATGGATCAACCCTGA
CAGTGGTGGCACAAACTATGCACAGAAGTTTCAGGGCAGGGTCACCATGACCAGGGA
CACGTCCATCAGCACAGCCTACATGGAGCTGAACAGGCTGAGATCTGACGACACGGC
CGTGTATTACTGTGCGAGAGATCAGCCCCTAGGATATTGTACTAATGGTGTATGCTCC
TACTTTGACTACTGGGGCCAGGGAACCCTGGTCACCGTCTCCTCAGGTGGAGGCGGTT
CAGGCGGAGGTGGCAGCGGCGGTGGCGGGTCGGACATCCAGATGACCCAGTCTCCAT
CTTCCGTGTCTGCATCTGTAGGAGACAGAGTCACCATCACTTGTCGGGCGAGTCAGGG
TATTTACAGCTGGTTAGCCTGGTATCAGCAGAAACCAGGGAAAGCCCCTAACCTCCTG
ATCTATACTGCATCCACTTTACAAAGTGGGGTCCCATCAAGGTTCAGCGGCAGTGGAT
CTGGGACAGATTTCACTCTCACCATCAGCAGCCTGCAACCTGAAGATTTTGCAACTTA
CTATTGTCAACAGGCTAACATTTTCCCGCTCACTTTCGGCGGAGGGACCAAGGTGGAG
ATCAAAGAGTCCAAATATGGTCCCCCATGCCCACCATGCCCAGCACCTGAGTTCCTGG
GGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGT
CAAAGGCTTCTACCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTAC
AGCAGGCTAACCGTGGACAAGAGCAGGTGGCAGGAGGGGAATGTCTTCTCATGCTCC
GTGATGCATGAGGCTCTGCACAACCACTACACACAGAAGAGCCTCTCCCTGTCTCTGG
GTAAACCCTTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGCTCCTGCAC
AGTGACTACATGAACATGACTCCCCGCCGCCCCGGGCCCACCCGCAAGCATTACCAG
CCCTATGCCCCACCACGCGACTTCGCAGCCTATCGCTCCAAACGGGGCAGAAAGAAG
CTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTG
68. Nucleic acid sequence of 40DCR4 (including signal peptide)
(SEQ ID NO: 68)
ATGGCCTTACCAGTGACCGCCTTGCTCCTGCCGCTGGCCTTGCTGCTCCACGCCG
CCAGGCCGAGCCAGGTGCAGCTGGTGCAGTCTGGGGCTGAGGTGAAGAAGCCTGGGG
CCTCAGTGAAGGTCTCCTGCAAGGCTTCTGGATACACCTTCACCGGCTACTATATGCA
CTGGGTGCGACAGGCCCCTGGACAAGGGCTTGAGTGGATGGGATGGATCAACCCTGA
CAGTGGTGGCACAAACTATGCACAGAAGTTTCAGGGCAGGGTCACCATGACCAGGGA
CACGTCCATCAGCACAGCCTACATGGAGCTGAACAGGCTGAGATCTGACGACACGGC
CGTGTATTACTGTGCGAGAGATCAGCCCCTAGGATATTGTACTAATGGTGTATGCTCC
TACTTTGACTACTGGGGCCAGGGAACCCTGGTCACCGTCTCCTCAGGTGGAGGCGGTT
CAGGCGGAGGTGGCAGCGGCGGTGGCGGGTCGGACATCCAGATGACCCAGTCTCCAT
CTTCCGTGTCTGCATCTGTAGGAGACAGAGTCACCATCACTTGTCGGGCGAGTCAGGG
TATTTACAGCTGGTTAGCCTGGTATCAGCAGAAACCAGGGAAAGCCCCTAACCTCCTG
ATCTATACTGCATCCACTTTACAAAGTGGGGTCCCATCAAGGTTCAGCGGCAGTGGAT
CTGGGACAGATTTCACTCTCACCATCAGCAGCCTGCAACCTGAAGATTTTGCAACTTA
CTATTGTCAACAGGCTAACATTTTCCCGCTCACTTTCGGCGGAGGGACCAAGGTGGAG
ATCAAAGAGTCCAAATATGGTCCCCCATGCCCACCATGCCCAGCACCTGAGTTCCTGG
GGGGACCATCAGTCTTCCTGTTCCCCCCAAAACCCAAGGACACTCTCATGATCTCCCG
GACCCCTGAGGTCACGTGCGTGGTGGTGGACGTGAGCCAGGAAGACCCCGAGGTCCA
GTTCAACTGGTACGTGGATGGCGTGGAGGTGCATAATGCCAAGACAAAGCCGCGGGA
GGAGCAGTTCAACAGCACGTACCGTGTGGTCAGCGTCCTCACCGTCCTGCACCAGGA
CTGGCTGAACGGCAAGGAGTACAAGTGCAAGGTCTCCAACAAAGGCCTCCCGTCCTC
CATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGT
CAAAGGCTTCTACCCCAGCGACATCGCCGTGGAGTGGGAGAGCAATGGGCAGCCGGA
GAACAACTACAAGACCACGCCTCCCGTGCTGGACTCCGACGGCTCCTTCTTCCTCTAC
AGCAGGCTAACCGTGGACAAGAGCAGGTGGCAGGAGGGGAATGTCTTCTCATGCTCC
GTGATGCATGAGGCTCTGCACAACCACTACACACAGAAGAGCCTCTCCCTGTCTCTGG
GTAAACCCTTTTGGGTGCTGGTGGTGGTTGGTGGAGTCCTGGCTTGCTATAGCTTGCT
AGTAACAGTGGCCTTTATTATTTTCTGGGTGAAACGGGGCAGAAAGAAGCTCCTGTAT
ATATTCAAACAACCATTTATGAGACCAGTACAAACTACTCAAGAGGAAGATGGCTGT
AGCTGCCGATTTCCAGAAGAAGAAGAAGGAGGATGTGAACTGAGGAGTAAGAGGAG
CAGGCTCCTGCACAGTGACTACATGAACATGACTCCCCGCCGCCCCGGGCCCACCCG
CAAGCATTACCAGCCCTATGCCCCACCACGCGACTTCGCAGCCTATCGCTCC
69. Nucleic acid sequence of 40DCR1 (without signal peptide)
(SEQ ID NO: 69)
CAGGTGCAGCTGGTGCAGTCTGGGGCTGAGGTGAAGAAGCCTGGGGCCTCAGTG
AAGGTCTCCTGCAAGGCTTCTGGATACACCTTCACCGGCTACTATATGCACTGGGTGC
GACAGGCCCCTGGACAAGGGCTTGAGTGGATGGGATGGATCAACCCTGACAGTGGTG
GCACAAACTATGCACAGAAGTTTCAGGGCAGGGTCACCATGACCAGGGACACGTCCA
TCAGCACAGCCTACATGGAGCTGAACAGGCTGAGATCTGACGACACGGCCGTGTATT
ACTGTGCGAGAGATCAGCCCCTAGGATATTGTACTAATGGTGTATGCTCCTACTTTGA
CTACTGGGGCCAGGGAACCCTGGTCACCGTCTCCTCAGGTGGAGGCGGTTCAGGCGG
AGGTGGCAGCGGCGGTGGCGGGTCGGACATCCAGATGACCCAGTCTCCATCTTCCGT
GTCTGCATCTGTAGGAGACAGAGTCACCATCACTTGTCGGGCGAGTCAGGGTATTTAC
AGCTGGTTAGCCTGGTATCAGCAGAAACCAGGGAAAGCCCCTAACCTCCTGATCTAT
ACTGCATCCACTTTACAAAGTGGGGTCCCATCAAGGTTCAGCGGCAGTGGATCTGGG
ACAGATTTCACTCTCACCATCAGCAGCCTGCAACCTGAAGATTTTGCAACTTACTATT
GTCAACAGGCTAACATTTTCCCGCTCACTTTCGGCGGAGGGACCAAGGTGGAGATCA
AAGAGTCCAAATATGGTCCCCCATGCCCACCATGCCCAGCACCTGAGTTCCTGGGGG
GACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTG
GCTGAACGGCAAGGAGTACAAGTGCAAGGTCTCCAACAAAGGCCTCCCGTCCTCCAT
CGAGAAAACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AACCCTTTTGGGTGCTGGTGGTGGTTGGTGGAGTCCTGGCTTGCTATAGCTTGCTAGT
AACAGTGGCCTTTATTATTTTCTGGGTGAGGAGTAAGAGGAGCAGGCTCCTGCACAGT
GACTACATGAACATGACTCCCCGCCGCCCCGGGCCCACCCGCAAGCATTACCAGCCC
TATGCCCCACCACGCGACTTCGCAGCCTATCGCTCC
70. Nucleic acid sequence of 28DCR2 (without signal peptide)
(SEQ ID NO: 70)
CAGGTGCAGCTGGTGCAGTCTGGGGCTGAGGTGAAGAAGCCTGGGGCCTCAGTG
AAGGTCTCCTGCAAGGCTTCTGGATACACCTTCACCGGCTACTATATGCACTGGGTGC
GACAGGCCCCTGGACAAGGGCTTGAGTGGATGGGATGGATCAACCCTGACAGTGGTG
GCACAAACTATGCACAGAAGTTTCAGGGCAGGGTCACCATGACCAGGGACACGTCCA
TCAGCACAGCCTACATGGAGCTGAACAGGCTGAGATCTGACGACACGGCCGTGTATT
ACTGTGCGAGAGATCAGCCCCTAGGATATTGTACTAATGGTGTATGCTCCTACTTTGA
CTACTGGGGCCAGGGAACCCTGGTCACCGTCTCCTCAGGTGGAGGCGGTTCAGGCGG
AGGTGGCAGCGGCGGTGGCGGGTCGGACATCCAGATGACCCAGTCTCCATCTTCCGT
GTCTGCATCTGTAGGAGACAGAGTCACCATCACTTGTCGGGCGAGTCAGGGTATTTAC
AGCTGGTTAGCCTGGTATCAGCAGAAACCAGGGAAAGCCCCTAACCTCCTGATCTAT
ACTGCATCCACTTTACAAAGTGGGGTCCCATCAAGGTTCAGCGGCAGTGGATCTGGG
ACAGATTTCACTCTCACCATCAGCAGCCTGCAACCTGAAGATTTTGCAACTTACTATT
GTCAACAGGCTAACATTTTCCCGCTCACTTTCGGCGGAGGGACCAAGGTGGAGATCA
AAGAGTCCAAATATGGTCCCCCATGCCCACCATGCCCAGCACCTGAGTTCCTGGGGG
GACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTG
GCTGAACGGCAAGGAGTACAAGTGCAAGGTCTCCAACAAAGGCCTCCCGTCCTCCAT
CGAGAAAACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AACCCTTTTGGGTGCTGGTGGTGGTTGGTGGAGTCCTGGCTTGCTATAGCTTGCTAGT
AACAGTGGCCTTTATTATTTTCTGGGTGAGGAGTAAGAGGAGCAGGCTCCTGCACAGT
GACTACATGAACATGACTCCCCGCCGCCCCGGGCCCACCCGCAAGCATTACCAGCCC
TATGCCCCACCACGCGACTTCGCAGCCTATCGCTCC
71. Nucleic acid sequence of 40DCR3 (without signal peptide)
(SEQ ID NO: 71)
CAGGTGCAGCTGGTGCAGTCTGGGGCTGAGGTGAAGAAGCCTGGGGCCTCAGTG
AAGGTCTCCTGCAAGGCTTCTGGATACACCTTCACCGGCTACTATATGCACTGGGTGC
GACAGGCCCCTGGACAAGGGCTTGAGTGGATGGGATGGATCAACCCTGACAGTGGTG
GCACAAACTATGCACAGAAGTTTCAGGGCAGGGTCACCATGACCAGGGACACGTCCA
TCAGCACAGCCTACATGGAGCTGAACAGGCTGAGATCTGACGACACGGCCGTGTATT
ACTGTGCGAGAGATCAGCCCCTAGGATATTGTACTAATGGTGTATGCTCCTACTTTGA
CTACTGGGGCCAGGGAACCCTGGTCACCGTCTCCTCAGGTGGAGGCGGTTCAGGCGG
AGGTGGCAGCGGCGGTGGCGGGTCGGACATCCAGATGACCCAGTCTCCATCTTCCGT
GTCTGCATCTGTAGGAGACAGAGTCACCATCACTTGTCGGGCGAGTCAGGGTATTTAC
AGCTGGTTAGCCTGGTATCAGCAGAAACCAGGGAAAGCCCCTAACCTCCTGATCTAT
ACTGCATCCACTTTACAAAGTGGGGTCCCATCAAGGTTCAGCGGCAGTGGATCTGGG
ACAGATTTCACTCTCACCATCAGCAGCCTGCAACCTGAAGATTTTGCAACTTACTATT
GTCAACAGGCTAACATTTTCCCGCTCACTTTCGGCGGAGGGACCAAGGTGGAGATCA
AAGAGTCCAAATATGGTCCCCCATGCCCACCATGCCCAGCACCTGAGTTCCTGGGGG
GACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTG
GCTGAACGGCAAGGAGTACAAGTGCAAGGTCTCCAACAAAGGCCTCCCGTCCTCCAT
CGAGAAAACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AACCCTTTTGGGTGCTGGTGGTGGTTGGTGGAGTCCTGGCTTGCTATAGCTTGCTAGT
AACAGTGGCCTTTATTATTTTCTGGGTGAGGAGTAAGAGGAGCAGGCTCCTGCACAGT
GACTACATGAACATGACTCCCCGCCGCCCCGGGCCCACCCGCAAGCATTACCAGCCC
TATGCCCCACCACGCGACTTCGCAGCCTATCGCTCCAAACGGGGCAGAAAGAAGCTC
CTGTATATATTCAAACAACCATTTATGAGACCAGTACAAACTACTCAAGAGGAAGAT
GGCTGTAGCTGCCGATTTCCAGAAGAAGAAGAAGGAGGATGTGAACTG
72. Nucleic acid sequence of 40DCR4 (without signal peptide)
(SEQ ID NO: 72)
CAGGTGCAGCTGGTGCAGTCTGGGGCTGAGGTGAAGAAGCCTGGGGCCTCAGTG
AAGGTCTCCTGCAAGGCTTCTGGATACACCTTCACCGGCTACTATATGCACTGGGTGC
GACAGGCCCCTGGACAAGGGCTTGAGTGGATGGGATGGATCAACCCTGACAGTGGTG
GCACAAACTATGCACAGAAGTTTCAGGGCAGGGTCACCATGACCAGGGACACGTCCA
TCAGCACAGCCTACATGGAGCTGAACAGGCTGAGATCTGACGACACGGCCGTGTATT
ACTGTGCGAGAGATCAGCCCCTAGGATATTGTACTAATGGTGTATGCTCCTACTTTGA
CTACTGGGGCCAGGGAACCCTGGTCACCGTCTCCTCAGGTGGAGGCGGTTCAGGCGG
AGGTGGCAGCGGCGGTGGCGGGTCGGACATCCAGATGACCCAGTCTCCATCTTCCGT
GTCTGCATCTGTAGGAGACAGAGTCACCATCACTTGTCGGGCGAGTCAGGGTATTTAC
AGCTGGTTAGCCTGGTATCAGCAGAAACCAGGGAAAGCCCCTAACCTCCTGATCTAT
ACTGCATCCACTTTACAAAGTGGGGTCCCATCAAGGTTCAGCGGCAGTGGATCTGGG
ACAGATTTCACTCTCACCATCAGCAGCCTGCAACCTGAAGATTTTGCAACTTACTATT
GTCAACAGGCTAACATTTTCCCGCTCACTTTCGGCGGAGGGACCAAGGTGGAGATCA
AAGAGTCCAAATATGGTCCCCCATGCCCACCATGCCCAGCACCTGAGTTCCTGGGGG
GACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTT
CAACTGGTACGTGGATGGCGTGGAGGTGCATAATGCCAAGACAAAGCCGCGGGAGG
AGCAGTTCAACAGCACGTACCGTGTGGTCAGCGTCCTCACCGTCCTGCACCAGGACTG
GCTGAACGGCAAGGAGTACAAGTGCAAGGTCTCCAACAAAGGCCTCCCGTCCTCCAT
CGAGAAAACCATCTCCAAAGCCAAAGGGCAGCCCCGAGAGCCACAGGTGTACACCCT
GCCCCCATCCCAGGAGGAGATGACCAAGAACCAGGTCAGCCTGACCTGCCTGGTCAA
AGGCTTCTACCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTACAGC
AGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTA
AACCCTTTTGGGTGCTGGTGGTGGTTGGTGGAGTCCTGGCTTGCTATAGCTTGCTAGT
AACAGTGGCCTTTATTATTTTCTGGGTGAAACGGGGCAGAAAGAAGCTCCTGTATATA
TTCAAACAACCATTTATGAGACCAGTACAAACTACTCAAGAGGAAGATGGCTGTAGC
TGCCGATTTCCAGAAGAAGAAGAAGGAGGATGTGAACTGAGGAGTAAGAGGAGCAG
GCTCCTGCACAGTGACTACATGAACATGACTCCCCGCCGCCCCGGGCCCACCCGCAA
GCATTACCAGCCCTATGCCCCACCACGCGACTTCGCAGCCTATCGCTCC
73. Amino acid sequence of EGFR G1 CAR
(SEQ ID NO: 73)
ATMALPVTALLLPLALLLHAARPSDILLTQSPVILSVSPGERVSFSCRASQSIGTNIHW
YQQRTNGSPRLLIKYASESISGIPSRFSGSGSGTDFTLSINSVESEDIADYYCQQNNNWPTTF
GAGTKLELKGGGGSGGGGSGGGGSQVQLKQSGPGLVQPSQSLSITCTVSGFSLTNYGVH
WVRQSPGKGLEWLGVIWSGGNTDYNTPFTSRLSINKDNSKSQVFFKMNSLQSNDTAIYY
CARALTYYDYEFAYWGQGTLVTVSSFVPVFLPAKPTTTPAPRPPTPAPTIASQPLSLRPEA
CRPAAGGAVHTRGLDFACDIYIWAPLAGTCGVLLLSLVITLYCNHRSKRSRLLHSDYMN
MTPRRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGHDGL
YQGLSTATKDTYDALHMQALPPR
74. Nucleic acid sequence of EGFR G1 CAR
(SEQ ID NO: 74)
GCCACCATGGCCTTACCAGTGACCGCCTTGCTCCTGCCGCTGGCCTTGCTGCTCC
ACGCCGCCAGGCCGAGCGACATCTTGCTGACTCAGTCTCCAGTCATCCTGTCTGTGAG
TCCAGGAGAAAGAGTCAGTTTCTCCTGCAGGGCCAGTCAGAGTATTGGCACAAACAT
ACACTGGTATCAGCAAAGAACAAATGGTTCTCCAAGGCTTCTCATAAAGTATGCTTCT
GAGTCTATCTCTGGGATCCCTTCCAGGTTTAGTGGCAGTGGATCAGGGACAGATTTTA
CTCTTAGCATCAACAGTGTGGAGTCTGAAGATATTGCAGATTATTACTGTCAACAAAA
TAATAACTGGCCAACCACGTTCGGTGCTGGGACCAAGCTGGAGCTGAAAGGTGGAGG
CGGTTCAGGCGGAGGTGGCAGCGGCGGTGGCGGGTCGCAGGTGCAGCTGAAGCAGTC
AGGACCTGGCCTAGTGCAGCCCTCACAGAGCCTGTCCATCACCTGCACAGTCTCTGGT
TTCTCATTAACTAACTATGGTGTACACTGGGTTCGCCAGTCTCCAGGAAAGGGTCTGG
AGTGGCTGGGAGTGATATGGAGTGGTGGAAACACAGACTATAATACACCTTTCACAT
CCAGACTGAGCATCAACAAGGACAATTCCAAGAGCCAAGTTTTCTTTAAAATGAACA
GTCTGCAATCTAATGACACAGCCATATATTACTGTGCCAGAGCCCTCACCTACTATGA
TTACGAGTTTGCTTACTGGGGCCAAGGGACTCTGGTCACTGTCTCTTCGTTCGTGCCG
GTCTTCCTGCCAGCGAAGCCCACCACGACGCCAGCGCCGCGACCACCAACACCGGCG
CCCACCATCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACATCTGGGCG
CCCCTGGCCGGGACTTGTGGGGTCCTTCTCCTGTCACTGGTTATCACCCTTTACTGCAA
CCACAGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTTCGCA
GCCTATCGCTCCAGAGTGAAGTTCAGCAGGAGCGCAGACGCCCCCGCGTACCAGCAG
GGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTGC
CCCCTCGCTGATAA
75. Nucleotide sequence of EGFR G2 CAR
(SEQ ID NO: 75)
GCCACCATGGCCTTACCAGTGACCGCCTTGCTCCTGCCGCTGGCCTTGCTGCTCC
ACGCCGCCAGGCCGAGCGACATCTTGCTGACTCAGTCTCCAGTCATCCTGTCTGTGAG
TCCAGGAGAAAGAGTCAGTTTCTCCTGCAGGGCCAGTCAGAGTATTGGCACAAACAT
ACACTGGTATCAGCAAAGAACAAATGGTTCTCCAAGGCTTCTCATAAAGTATGCTTCT
GAGTCTATCTCTGGGATCCCTTCCAGGTTTAGTGGCAGTGGATCAGGGACAGATTTTA
CTCTTAGCATCAACAGTGTGGAGTCTGAAGATATTGCAGATTATTACTGTCAACAAAA
TAATAACTGGCCAACCACGTTCGGTGCTGGGACCAAGCTGGAGCTGAAAGGTGGAGG
CGGTTCAGGCGGAGGTGGCAGCGGCGGTGGCGGGTCGCAGGTGCAGCTGAAGCAGTC
AGGACCTGGCCTAGTGCAGCCCTCACAGAGCCTGTCCATCACCTGCACAGTCTCTGGT
TTCTCATTAACTAACTATGGTGTACACTGGGTTCGCCAGTCTCCAGGAAAGGGTCTGG
AGTGGCTGGGAGTGATATGGAGTGGTGGAAACACAGACTATAATACACCTTTCACAT
CCAGACTGAGCATCAACAAGGACAATTCCAAGAGCCAAGTTTTCTTTAAAATGAACA
GTCTGCAATCTAATGACACAGCCATATATTACTGTGCCAGAGCCCTCACCTACTATGA
TTACGAGTTTGCTTACTGGGGCCAAGGGACTCTGGTCACTGTCTCTTCGTTCGTGCCG
GTCTTCCTGCCAGCGAAGCCCACCACGACGCCAGCGCCGCGACCACCAACACCGGCG
CCCACCATCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACATCTGGGCG
CCCCTGGCCGGGACTTGTGGGGTCCTTCTCCTGTCACTGGTTATCACCCTTTACTGCAA
CCACAGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTTCGCA
GCCTATCGCTCCAGAGTGAAGTTCAGCAGGAGCGCAGACGCCCCCGCGTACCAGCAG
GGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTACAG
TGAGATTGGGATGAAAGGCGAGCGCCGGAGGGGCAAGGGGCACGATGGCCTTTACC
AGGGTCTCAGTACAGCCACCAAGGACACCTACGACGCCCTTCACATGCAGGCCCTGC
CCCCTCGCTGATAA
76. Nucleotide sequence of primer CD4O-F
(SEQ ID NO: 76)
ACCTCCTGATCTATACTG
77. Nucleotide sequence of primer CD4O-R
(SEQ ID NO: 77)
GATGGTGAGAGTGAAATC
78. Nucleotide sequence in probe Taqman
(SEQ ID NO: 78)
CACTGCCGCTGAACCTTGATG

### DETAILED DESCRIPTION

The embodiments of the present disclosure illustrated by way of specific examples below.

Those skilled in the art will understand that these examples are merely exemplary and should not be considered as limiting the scope of the present disclosure. The experimental methods without specifying the specific technology or conditions in the following examples generally used the conventional conditions, such as those described in Sambrook. J., Molecular Cloning: A Laboratory Manual (3rd ed.), translated by Huang Peitang, Science Press, or followed the manufacturer's recommendation. The used reagents or instruments without specifying the manufacturer are all conventional products that are commercially available.

### Example 1-(1): Construction of 5 Recombinant Plasmids: pNB328-Meso CAR G1, pNB328-meso G2 CAR, PS328b 137 DCR1, PS328b 137 DCR2 and PS328b 137 DCR3

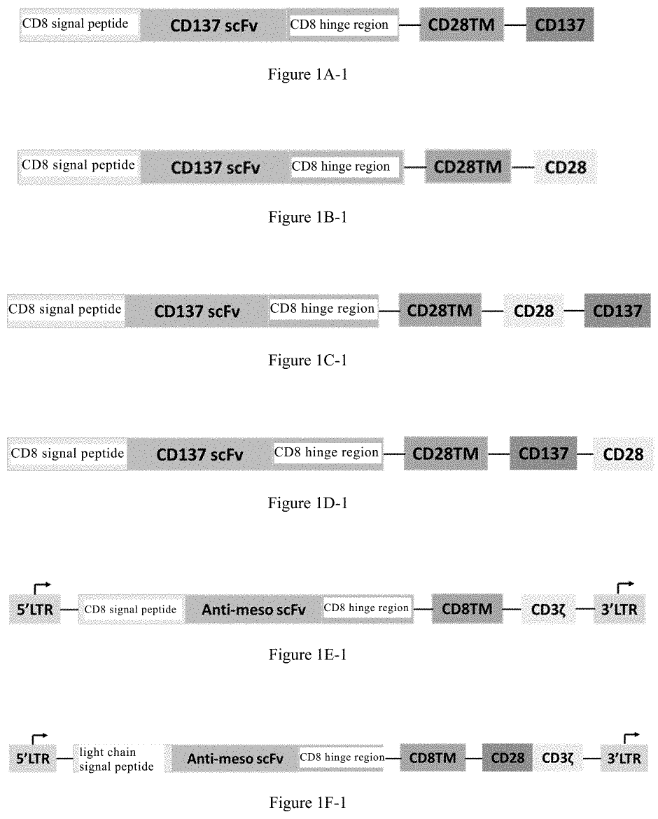

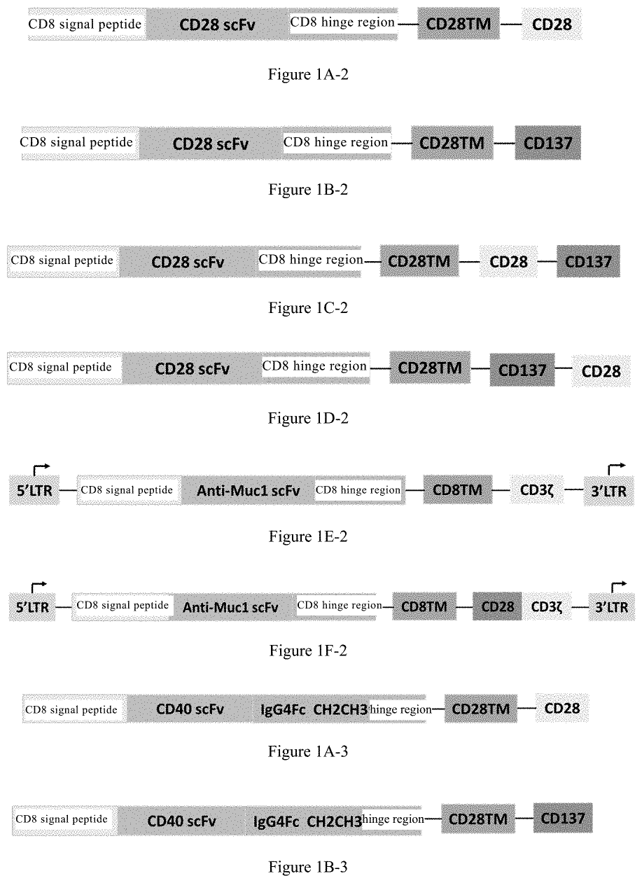

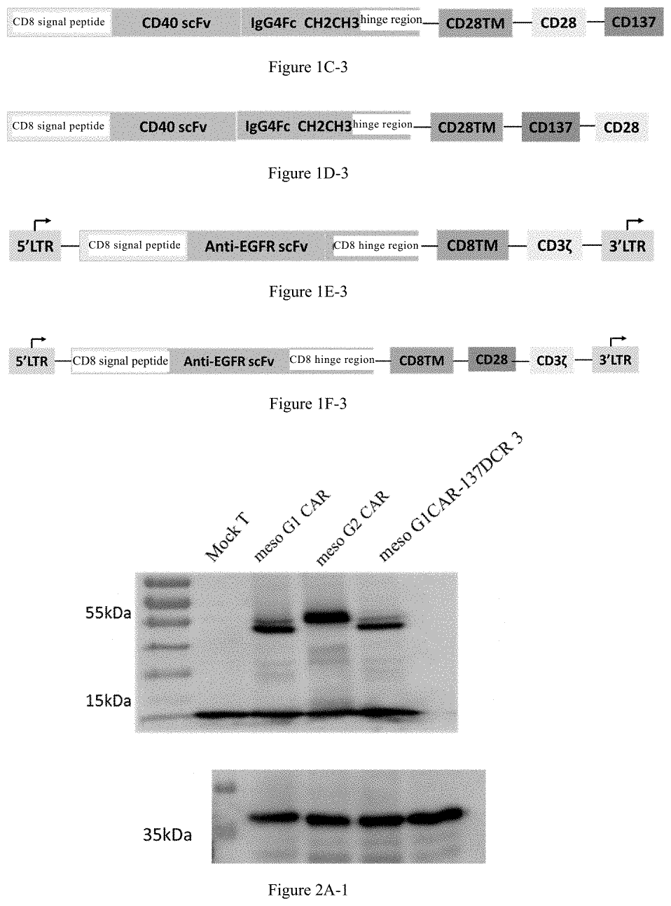

1. 137DCR1 gene (SEQ ID NO: 15), 137DCR2 gene (SEQ ID NO: 16), 137DCR3 gene (SEQ ID NO: 17), meso G1 CAR gene (SEQ ID NO: 24) and meso G2 CAR Gene (SEQ ID NO: 26) were synthesized artificially, and diagrams of their structures are shown in FIG. 1A-1, FIG. 1B-1, FIG. 1C-1, FIG. 1E-1, and FIG. 1F-1, respectively. The synthesized 5 genes were inserted into PNB328 vector and PS328b vector, between EcoRI and SalI restriction sites.

pNB328 vector contains EF1α promoter, PB transposon and other elements, which is constructed according to Example 2 of WO2017054647A1. PS328b is an artificially synthesized sequence, synthesized by Shanghai Generay Biological Engineering Co., Ltd., with sequence shown in SEQ ID NO:27.

The constructed recombinant plasmids were named pNB328-meso G1 CAR plasmid, pNB328-meso G2 CAR plasmid, PS328b 137DCR1 plasmid, PS328b 137DCR2 plasmid and PS328b 137DCR3 plasmid. The constructed recombinant plasmids can carry foreign genes and be integrated into the host cell genome.

### Example 1-(2): Construction of 5 Recombinant Plasmids: pNB328-Muc1 G1 CAR, pNB328-Muc1 G2 CAR, PS328b 28DCR1, PS328b 28DCR2 and PS328b 28DCR3

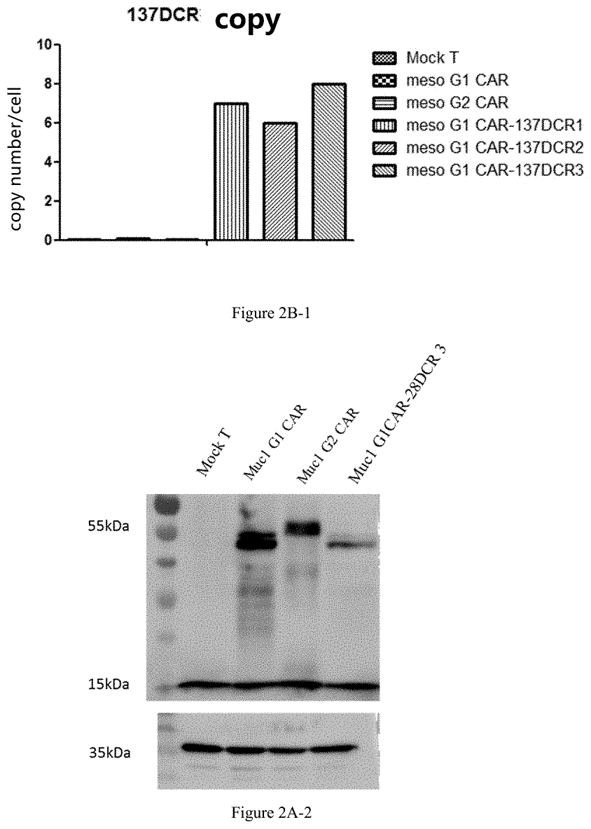

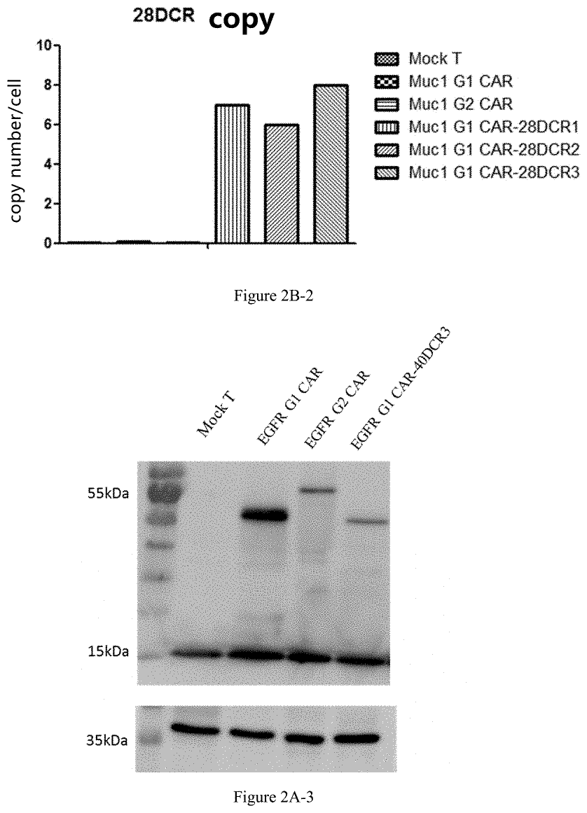

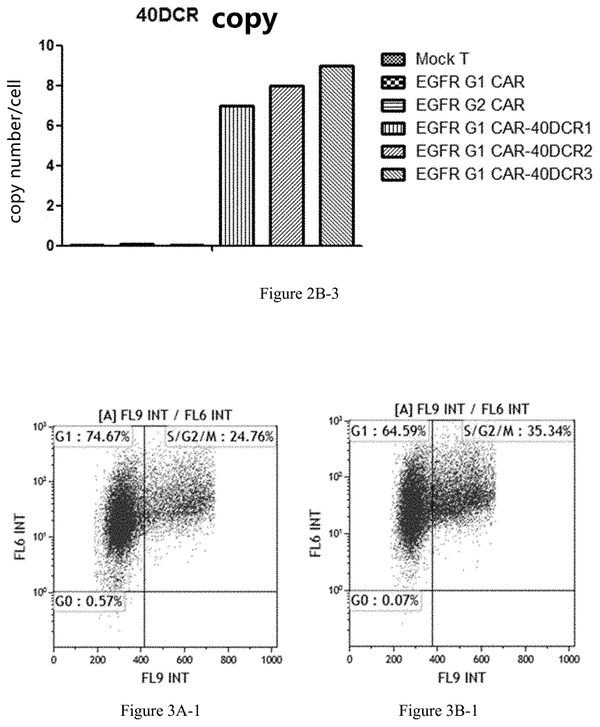

1. 28DCR1 gene (SEQ ID NO: 40), 28DCR2 gene (SEQ ID NO: 41), 28DCR3 gene (SEQ ID NO: 42), Muc1 G1 CAR gene (SEQ ID NO: 49) and Muc1 G2 CAR Gene (SEQ ID NO: 50) were synthesized artificially, and diagrams of their structures are shown in FIG. 1A-2, FIG. 1B-2, FIG. 1C-2, FIG. 1E-2, and FIG. 1F-2, respectively. The synthesized 5 genes were inserted into PNB328 vector and PS328b vector, between EcoRI and SalI restriction sites.

pNB328 vector contains EF1α promoter, PB transposon and other elements, which is constructed according to Example 2 of WO2017054647A1. PS328b is an artificially synthesized sequence, synthesized by Shanghai Generay Biological Engineering Co., Ltd., with sequence shown in SEQ ID NO:27.

The constructed recombinant plasmids were named pNB328-Muc1 G1 CAR plasmid, pNB328-Muc1 G2 CAR plasmid, PS328b 28DCR1 plasmid, PS328b 28DCR2 plasmid and PS328b 28DCR3 plasmid. The constructed recombinant plasmids can carry foreign genes and be integrated into the host cell genome.

### Example 1-(3): Construction of 5 Recombinant Plasmids: pNB328-EGFR G1 CAR, pNB328-EGFR G2 CAR, PS328b 40DCR1, PS328b 40DCR2 and PS328b 40DCR3

1. 40DCR1 gene (SEQ ID NO: 65), 40DCR2 gene (SEQ ID NO: 66), 40DCR3 gene (SEQ ID NO: 67), EGFR G1 CAR gene (SEQ ID NO: 74) and EGFR G2 CAR Gene (SEQ ID NO: 75) were synthesized artificially, and diagrams of their structures are shown in FIG. 1A-3, FIG. 1B-3, FIG. 1C-3, FIG. 1F-3, and FIG. 1D-3, respectively. The synthesized 5 genes were inserted into pNB328 vector and PS328b vector, between EcoRI and SalI restriction sites.

pNB328 vector contains EF1α promoter, PB transposon and other elements, which is constructed according to Example 2 of WO2017054647A1. PS328b is an artificially synthesized sequence, synthesized by Shanghai Generay Biological Engineering Co., Ltd., with sequence shown in SEQ ID NO:27.

The constructed recombinant plasmids were named pNB328-EGFR G1 CAR plasmid, pNB328-EGFR G2 CAR plasmid, PS328b 40DCR1 plasmid, PS328b 40DCR2 plasmid and PS328b 40DCR3 plasmid. The constructed recombinant plasmids can carry foreign genes and be integrated into the host cell genome.

### Example 2-(1): Construction and Identification of 9 Chimeric Antigen Receptor Modified T Cells: Recombinant Cells Meso G1 CAR, Meso G2 CAR, Meso G1 CAR-137DCR1, Meso G1 CAR-137DCR2, Meso G1 CAR-137DCR3, 137DCR1, 137DCR2, 137DCR3 and Mock T

(1) Construction of 9 Recombinant Cells

Peripheral blood mononuclear cells (PBMCs) were adherently cultured for 2-4 hours, and the non-adherent suspension cells were the naive T cells. The suspension cells were collected in a 15 ml centrifuge tube, centrifuged at 1200 rmp for 3 min, and the supernatant was discarded; normal saline was added, centrifuged at 1200 rmp for 3 min, and the normal saline was discarded, and the steps of “normal saline was added, centrifuged at 1200 rmp for 3 min, and the normal saline was discarded” were repeat for three times.

5×10⁶ of the above cells were added to each of eight 1.5 ml centrifuge tubes, numbered a, b, c, d, e, f, g, h, centrifuged at 1200 rmp for 3 min, the supernatant was discarded and 100 μl electroporation reagent of the Electroporation Kit (Lonza) was added to each tube in proportion, wherein:

tube a: add 8 μg of pNB328-meso G1 CAR plasmid,

tube b: add 8 μg of pNB328-meso G2 CAR plasmid,

tube c: add 4 μg of each of PS328b 137DCR1 plasmid and pNB328-meso G1 CAR plasmid,

tube d: add 4 μg of each of PS328b 137DCR2 plasmid and pNB328-meso G1 CAR plasmid,

tube e: add 4 μg of each of PS328b 137DCR3 plasmid and pNB328-meso G1 CAR plasmid,

tube f: add 8 μg of PS328b 137DCR1 plasmid,

tube g: add 8 μg of PS328b 137DCR2 plasmid,

tube h: add 8 μg of PS328b 137DCR3 plasmid,

Each of the above 8 tubes was re-suspended and mixed to obtain mixtures.

Each of the mixture was transferred to an electroporation cup, which was put into the electroporation instrument, the required program was selected for electrical shock; the micro pipette in the kit was used to transfer the electroporated cell suspension to a six-well plate with the medium (AIM-V medium containing 2% FBS), which was mixed well and cultured in a 37° C., 5% CO2 incubator for 6 hours; then stimulating factor IL-2 and meso/anti-CD28 was added, cultured at 37° C., 5% CO2 for 3 to 4 days, the growth of T cells was observed. The recombinant T cells expressing pNB328-meso G1 CAR, pNB328-meso G2 CAR, pNB328-meso G1 CAR-137DCR1, pNB328-meso G1 CAR-137DCR2, pNB328-meso G1 CAR-137DCR3, PS328b 137DCR1, PS328b 137DCR2 and PS328 137DCR3 gene are obtained and named recombinant cell meso G1 CAR, recombinant cell meso G2 CAR, recombinant cell meso G1 CAR-137DCR1, recombinant cell meso G1 CAR-137DCR2 and recombinant cell meso G1 CAR-137DCR3, recombinant cell 137DCR1, recombinant cell 137DCR2 and recombinant cell 137DCR3, respectively.

In addition, 5×10⁶ cells were added in another centrifuge tube (tube i), centrifuged at 1200 rmp for 3 min, the supernatant was discarded, the electroporation kit (purchased from Lonza) was taken, 100 μl electroporation reagent of the Electroporation Kit (Lonza) in proportion was added, and 8 μg control plasmid (PNB328) was added, the control T cell, Mock T, was constructed and obtained according to the method described above.

(2) Identification of Positive Recombinant Cells

\textcircled{1} Detection of Meso G1 CAR or Meso G2 CAR Gene Expression by Western Blot Method

The above-mentioned recombinant cells meso G1 CAR, meso G2 CAR, meso G1 CAR-137DCR1, meso G1CAR-137DCR2, meso G1 CAR-137DCR3 and Mock-T cells were collected, and washed twice with normal saline,

160 μl of cell lysate was added and placed on ice for 10 min; after the cells were fully lysed, centrifuged at 12000 rpm at 4° C. for 10 min, and the supernatant was collected. 40 μl of 5×loading Buffer was added, incubated at 100° C. for 10 min, then placed on ice for 5 min.

The expression of CD3 of the previously constructed recombinant cells was detected by Western blot etc., using CD3 antibody (Abcam), GAPDH antibody (Beyotime), HRP goat anti-mouse secondary antibody (Jackson). The results are shown in FIG. 2A-1.

The results show that CD3 is highly expressed in all of these constructed recombinant T cells.

\textcircled{2} Detection of 137DCR1-3 Gene Expression by RT-PCR

The genomic DNA of recombinant cells meso G1 CAR, meso G2 CAR, meso G1 CAR-137DCR1, meso G1 CAR-137DCR2, meso G1 CAR-137DCR3 and Mock-T was extracted (kit method) using the experimental procedure based on the attached instruction in the kit.

The DNA concentration of each recombinant cells was determined, and the expression level of 137DCR gene was detected by fluorescence real-time quantitative PCR. The reaction was: 95° C., 15 s; 95° C., 5 s; 60° C., 15 s; for 40 cycles.

The PCR reaction system (20 μl) was as follows:

Taqman: 10 μl

CD137-F: 0.4 μl

CD137-R: 0.4 μl

Cd137-probe: 0.2 μl

Actin mix: 1 μl

H2O: 7 μl

The primer sequences were as follows:

(SEQ ID NO: 28)
CD137-F: CGAGTCACCATATCAGTA
(SEQ ID NO: 29)
CD137-R: CGAAGTACCAGTCATAATTC
(SEQ ID NO: 30)
Taqman: 5′FAM-CCTCGCACAGTAATATACAGCCGT-Trama

The results are shown in FIG. 2B-1. The results show that the expression levels of 137DCR1, 137DCR2 or 137DCR3 genes in the recombinant cells meso G1 CAR-137DCR1, meso G1 CAR-137DCR2, meso G1 CAR-137DCR3 are very high.

### Example 2-(2): Construction and Identification of 9 Chimeric Antigen Receptor Modified T Cells: Recombinant Cells Muc1 G1 CAR, Muc1 G2 CAR, Muc1 G1 CAR-28DCR1, Muc1 G1 CAR-28DCR2, Muc1 G1 CAR-28DCR3, 28DCR1, 28DCR2, 28DCR3 and Mock T

(1) Construction of 9 Recombinant Cells

Peripheral blood mononuclear cells (PBMCs) were adherently cultured for 2-4 hours, and the non-adherent suspension cells were the naive T cells. The suspension cells were collected in a 15 ml centrifuge tube, centrifuged at 1200 rmp for 3 min, and the supernatant was discarded; normal saline was added, centrifuged at 1200 rmp for 3 min, and the normal saline was discarded, and the steps of “normal saline was added, centrifuged at 1200 rmp for 3 min, and the normal saline was discarded” were repeated for three times.

5×10⁶ of the above cells was added to each of eight 1.5 ml centrifuge tubes, numbered a, b, c, d, e, f, g, h, centrifuged at 1200 rmp for 3 min, the supernatant was discarded and 100 μl electroporation reagent of the Electroporation Kit (Lonza) was added to each tube in proportion, wherein:

tube a: add 8 μg of pNB328-Muc1 G1 CAR plasmid,

tube b: add 8 μg of pNB328-Muc1 G2 CAR plasmid,

tube c: add 4 μg of each of PS328b 28DCR1 plasmid and pNB328-Muc1 G1 CAR plasmid,

tube d: add 4 μg of each of PS328b 28DCR2 plasmid and pNB328-Muc1 G1 CAR plasmid,

tube e: add 4 μg of each of PS328b 28DCR3 plasmid and pNB328-Muc1 G1 CAR plasmid,

tube f: add 8 μg of PS328b 28DCR1 plasmid,

tube g: add 8 μg of PS328b 28DCR2 plasmid,

tube h: add 8 μg of PS328b 28DCR3 plasmid,

Each of the above 8 tubes was re-suspended and mixed to obtain mixtures.

The mixture was transferred to an electroporation cup which was put into the electroporation instrument, the required program was selected for electrical shock; the micro pipette in the kit was used to transfer the electroporated cell suspension to a six-well plate with the medium (AIM-V medium containing 2% FBS), mixed well and cultured in a 37° C., 5% CO2 incubator for 6 hours; then stimulating factor IL-2 and Muc1/anti-CD28 was added, cultured at 37° C., 5% CO2 for 3 to 4 days, the growth of T cells was observed. The recombinant T cells expressing pNB328-Muc1 G1 CAR, pNB328-Muc1 G2 CAR, pNB328-Muc1 G1 CAR-28DCR1, pNB328-Muc1 G1 CAR-28DCR2, pNB328-Muc1 G1 CAR-28DCR3, PS328b 28DCR1, PS328b 28DCR2 and PS328b 28DCR3 gene were obtained and named recombinant cell Muc1 G1 CAR, recombinant cell Muc1 G2 CAR, recombinant cell Muc1 G1 CAR-28DCR1, recombinant cell Muc1 G1 CAR-28DCR2 and recombinant cell Muc1 G1 CAR-28DCR3, recombinant cell 28DCR1, recombinant cell 28DCR2 and recombinant cell 28DCR3, respectively.

In addition, 5×10⁶ cells were added in another centrifuge tube (tube i), centrifuged at 1200 rmp for 3 min, the supernatant was discarded, the electroporation kit (purchased from Lonza) was taken, 100 μl electroporation reagent of the Electroporation Kit (Lonza) in proportion was added and 8 μg control plasmid (PNB328) was added, the control T cell, Mock T was obtained according to the method described above.

(2) Identification of Positive Recombinant Cells

\textcircled{1} Detection of Muc1 G1 CAR or Muc1 G2 CAR Gene Expression by Western Blot Method

The above-mentioned recombinant cells Muc1 G1 CAR, Muc1 G2 CAR, Muc1 G1 CAR-28DCR1, Muc1 G1CAR-28DCR2, Muc1 G1 CAR-28DCR3 and Mock-T cells were collected, washed twice with normal saline,

160 μl of cell lysate was added and placed on ice for 10 min; after the cells were fully lysed, centrifuged at 12000 rpm at 4° C. for 10 min, and the supernatant was collected. 40 μl of 5×loading Buffer was added, incubated at 100° C. for 10 min, then placed on ice for 5 min.

The expression of CD3ζ of the constructed recombinant cells was detected by Western blot etc., using CD3ζ antibody (Abcam), GAPDH antibody (Beyotime), HRP goat anti-mouse secondary antibody (Jackson). The results are shown in FIG. 2A-2.

The results show that CD3ζ is highly expressed in all of these constructed recombinant T cells.

\textcircled{2} Detection of 28DCR1-3 Gene Expression by RT-PCR

The genomic DNA of recombinant cells Muc1 G1 CAR, Muc1 G2 CAR, Muc1 G1 CAR-28DCR1, Muc1 G1 CAR-28DCR2, Muc1 G1 CAR-28DCR3 and Mock-T was extracted (kit method) using the experimental procedure based on the attached instruction in the kit.

The DNA concentration of each recombinant cells was determined, and the expression level of 28DCR gene was detected by fluorescence real-time quantitative PCR. The reaction was: 95° C., 15 s; 95° C., 5 s; 60° C., 15 s; for 40 cycles.

The PCR reaction system (200 was as follows:

Taqman: 10 μl

CD28-F: 0.4 μl

CD28-R: 0.4 μl

CD28-probe: 0.2 μl

Actin mix: 1 μl

H2O: 7 μl

The primer sequences were as follows:

(SEQ ID NO: 51)
CD28-F: GCTTCTGGATACACCTTC
(SEQ ID NO: 52)
CD28-R: CCTTGAACTTCTCATTATAGTTAG
(SEQ ID NO: 53)
Taqman: 5′FAM-AATACATCCAATCCACTCAAGCC-Trama

The results are shown in FIG. 2B-2. The results show that the expression levels of 28DCR1, 28DCR2 or 28DCR3 genes in the recombinant cells Muc1 G1 CAR-28DCR1, Muc1 G1 CAR-28DCR2, Muc1 G1 CAR-28DCR3 are very high.

### Example 2-(3): Construction and Identification of 9 Chimeric Antigen Receptor Modified T Cells: Recombinant Cells EGFR G1 CAR, EGFR G2 CAR, EGFR G1 CAR-40DCR1, EGFR G1 CAR-40DCR2, EGFR G1 CAR-40DCR3, 40DCR1, 40DCR2, 40DCR3 and Mock T

(1) Construction of 9 Recombinant Cells

Peripheral blood mononuclear cells (PBMCs) were adherently cultured for 2-4 hours, and the non-adherent suspension cells were the naive T cells. The suspension cells were collected in a 15 ml centrifuge tube, centrifuged at 1200 rmp for 3 min, and the supernatant was discarded; normal saline was added, centrifuged at 1200 rmp for 3 min, and the normal saline was discarded, and the steps of “normal saline was added, centrifuged at 1200 rmp for 3 min, and the normal saline was discarded” was repeated for three times.

5×10⁶ of the above cells were added to each of eight 1.5 ml centrifuge tubes, numbered a, b, c, d, e, f, g, h, centrifuged at 1200 rmp for 3 min, the supernatant was discarded and 100 μl electroporation reagent of the Electroporation Kit (Lonza) was added to each tube in proportion, wherein:

tube a: add 8 μg of pNB328-EGFR G1 CAR plasmid,

tube b: add 8 μg of pNB328-EGFR G2 CAR plasmid,

tube c: add 4 μg of each of PS328b 40DCR1 plasmid and pNB328-EGFR G1 CAR plasmid,

tube d: add 4 μg of each of PS328b 40DCR2 plasmid and pNB328-EGFR G1 CAR plasmid,

tube e: add 4 μg of each of PS328b 40DCR3 plasmid and pNB328-EGFR G1 CAR plasmid,

tube f: add 8 μg of PS328b 40DCR1 plasmid,

tube g: add 8 μg of PS328b 40DCR2 plasmid,

tube h: add 8 μg of PS328b 40DCR3 plasmid,

Each of the above 8 tubes was re-suspended and mixed to obtain mixtures.

The mixture was transferred to an electroporation cup which was put into the electroporation instrument, the required program was selected for electrical shock; the micro pipette in the kit was used to transfer the electroporated cell suspension to a six-well plate with the medium (AIM-V medium containing 2% FBS), mixed well and cultured in a 37° C., 5% CO2 incubator for 6 hours; then stimulating factor IL-2 and EGFR/anti-CD28 was added, cultured at 37° C., 5% CO2 for 3 to 4 days, the growth of T cells was observed. The recombinant T cells expressing pNB328-EGFR G1 CAR, pNB328-EGFR G2 CAR, pNB328-EGFR G1 CAR-40DCR1, pNB328-EGFR G1 CAR-40DCR2, pNB328-EGFR G1 CAR-40DCR3, PS328b 40DCR1, PS328b 40DCR2 and PS328 40DCR3 gene were obtained and named as recombinant cell EGFR G1 CAR, recombinant cell EGFR G2 CAR, recombinant cell EGFR G1 CAR-40DCR1, recombinant cell EGFR G1 CAR-40DCR2 and recombinant cell EGFR G1 CAR-40DCR3, recombinant cell 40DCR1, recombinant cell 40DCR2 and recombinant cell 40DCR3, respectively.

In addition, 5×10⁶ cells were added in another centrifuge tube (tube j), centrifuged at 1200 rmp for 3 min, the supernatant was discarded, the electroporation kit (purchased from Lonza) was taken, 100 μl electroporation reagent of the Electroporation Kit (Lonza) in proportion was added and 8 μg control plasmid (PNB328) was added, the control T cell, Mock T was obtained according to the method described above.

(2) Identification of Positive Recombinant Cells

\textcircled{1} Detection of EGFR G1 CAR or EGFR G2 CAR Gene Expression by Western Blot Method

The above-mentioned recombinant cells EGFR G1 CAR, EGFR G2 CAR, EGFR G1 CAR-40DCR1, EGFR G1CAR-40DCR2, EGFR G1 CAR-40DCR3 and Mock-T cells were collected, washed twice with normal saline,

160 μl of cell lysate was added and placed on ice for 10 min; after the cells were fully lysed, centrifuged at 12000 rpm at 4° C. for 10 min, and the supernatant was collected. 40 μl of 5×loading Buffer was added, incubated at 100° C. for 10 min, then placed on ice for 5 min.

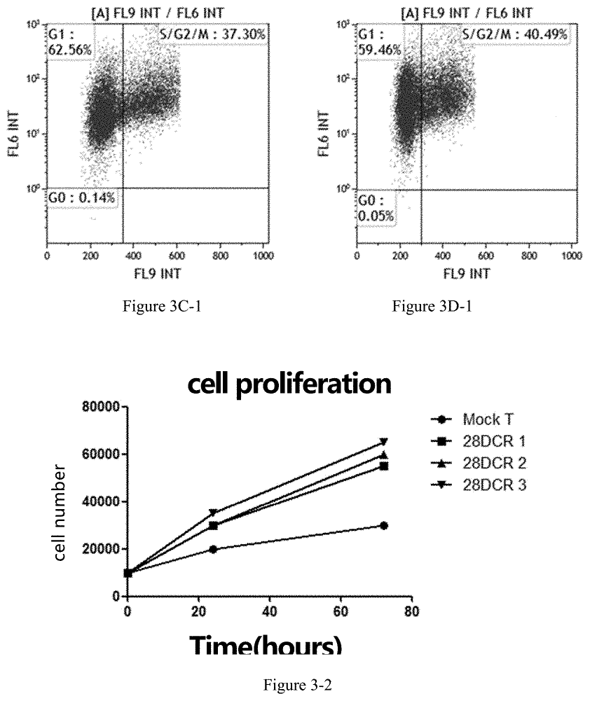

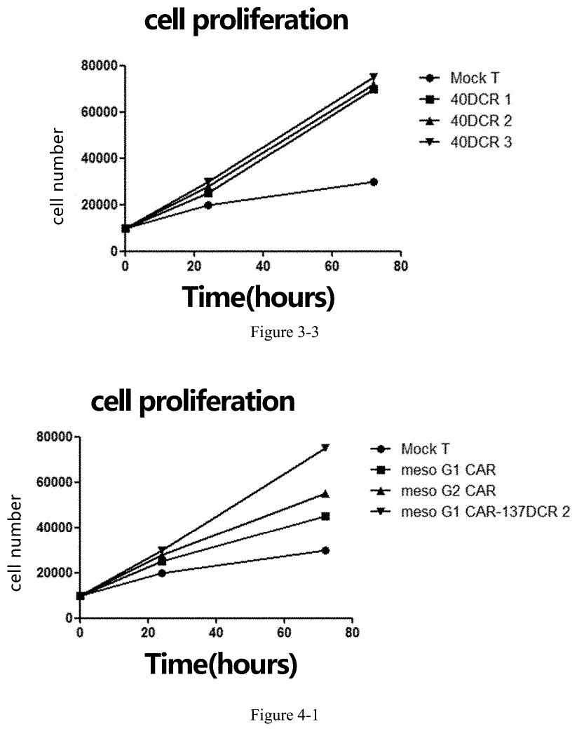

The expression of CD3 of the 5 constructed recombinant cells was detected by Western blot etc., using CD3ζ antibody (Abcam), GAPDH antibody (Beyotime), HRP goat anti-mouse secondary antibody (Jackson). The results are shown in FIG. 2A-3.

The results show that CD3 is highly expressed in all of these 5 constructed recombinant T cells.

\textcircled{2} Detection of 40DCR Gene Expression by RT-PCR

The genomic DNA of recombinant cells EGFR G1 CAR, EGFR G2 CAR, EGFR G1 CAR-40DCR1, EGFR G1 CAR-40DCR2, EGFR G1 CAR-40DCR3 and Mock-T was extracted (kit method) using the experimental procedure based on the attached instruction in the kit.

The DNA concentration of each recombinant cell was determined, and the expression level of 40DCR gene was detected by fluorescence real-time quantitative PCR. The reaction was: 95° C., 15 s; 95° C., 5 s; 60° C., 15 s; for 40 cycles.

The PCR reaction system (20 μl) was as follows:

Taqman: 10 μl

CD40-F: 0.4 μl

CD40-R: 0.4 μl

CD40-probe: 0.2 μl

Actin mix: 1 μl

H2O: 7 μl

The primer sequences were as follows:

(SEQ ID NO: 76)
CD40-F: ACCTCCTGATCTATACTG
(SEQ ID NO: 77)
CD40-R: GATGGTGAGAGTGAAATC
(SEQ ID NO: 78)
Taqman: 5′FAM-CACTGCCGCTGAACCTTGATG-Trama

The results are shown in FIG. 2B-3. The results show that the expression levels of 40DCR genes in the recombinant cells EGFR G1 CAR-137DCR1, EGFR G1 CAR-137DCR2, and EGFR G1 CAR-137DCR3 are very high.

### Example 3-(1): Detection of Cell Proliferation Activity by Flow Cytometry

1. Experimental Samples, Reagents and Instruments

The recombinant cells 137DCR1, 137DCR2, 137DCR3 and MockT prepared according to Example 2-(1).

2% FBS-containing PBS (1 ml Hyclone FBS+49 ml PBS) was formulated in a 50 ml centrifuge tube; 10×BD Perm/Wash™ buffer was diluted by 10-folds with ddH₂O, and placed on ice; Hoechst 33342 stock solution was diluted into working solution at 1:100 with ddH₂O (1 W/test).

Low temperature centrifuge (4° C. pre-cooling).

2. Experimental Method

All reagents were kept on ice during the experiment.

Steps were as follows:

(1) 1×10⁶-2×10⁶ of each of the above cells was collected in a 1.5 ml centrifuge tube, an appropriate amount of 2% FBS-containing PBS was added, centrifuged at 5000 rpm, 4° C. for 5 min, and the supernatant was discarded;

(2) the cells were re-suspended with 100 μl Fixation/Permeabilization Solution; the resuspension should be sufficient and gentle; the cells were fixed and permeabilized at 4° C. for 20 min;

(3) 1 ml of Perm/Wash™ buffer of the working concentration was added, mixed well, centrifuged at 7000 rpm, 4° C. for 5 min, the supernatant was discarded, and washed again;

(4) the cells were re-suspended with 100 μl of Perm/Wash™ buffer of the working concentration. 2-3 μl of Ki-67-APC antibody was added, and incubated at 4° C. in dark for 30 min;

(5) 1 ml of Perm/Wash™ buffer of working concentration was added, mixed well, centrifuged at 7000 rpm, 4° C. for 5 min, the supernatant was discarded, and washed again;

(6) the cells were re-suspended with 100 μl of Perm/Wash™ buffer of the working concentration. 1 μl Hoechst 33342 working solution was added and incubated on ice for 15 min;

(7) 400 μl of Perm/Wash™ buffer of the working concentration was further added, mixed well, and detected on the machine.

3. Experimental Results

The results are shown in FIGS. 3A-1 to 3D-1.

The results show that MockT has the slowest proliferation rate, 137DCR1 has a slower proliferation rate, 137DCR2 has a faster proliferation rate, and 137DCR3 has the fastest proliferation rate.

### Example 4-(1): Detection of Cell Proliferation Activity by Cell Proliferation Activity Kit

1. Experimental Samples and Reagents

The recombinant cells meso G1 CAR, meso G2 CAR, meso G1 CAR-137DCR2 and MockT prepared according to Example 2-(1).

CellTiter-Glo® Luminescent Cell Viability Assay, Promega, Cat. #G7570.

2. Experimental Method

(1) The above cells that have been cultured for 8 days were added in a 96-well white plate in 100 μL of AIM-V medium (containing cells) for each well.

(2) A blank control without cells was used to obtain the background fluorescence value.

(3) the complexes to be tested were added to the plate and incubated for 30 min in an incubator.

(4) 100 μL of CellTiter-Glo reagent was added, mixed well on an oscillator for 2 minutes, and incubated at room temperature for 10 minutes, then readings were obtained.

(5) Detections were made according to the above steps on the 8th, 9th, and 10th day of the culture.

3. Experimental Results

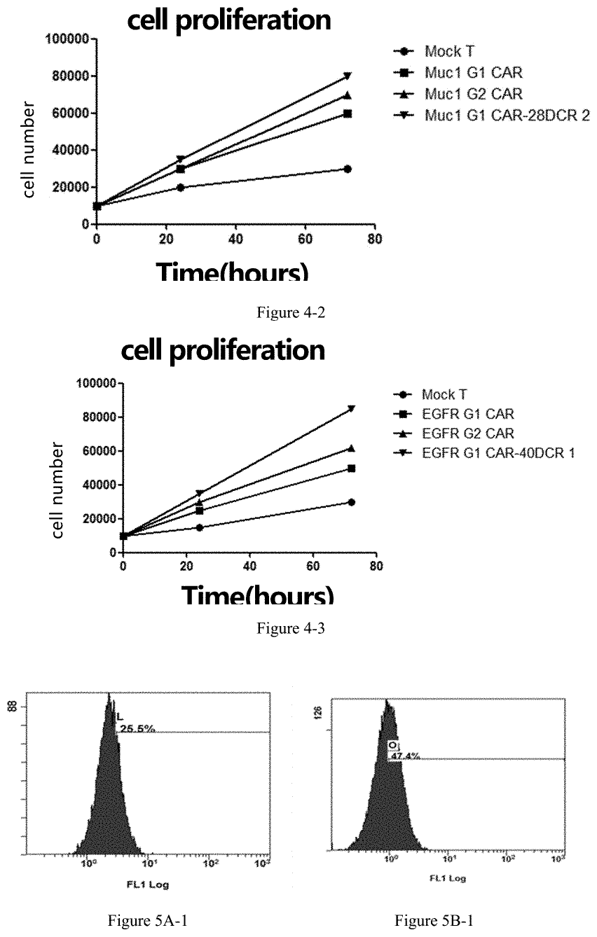

The results are shown in FIG. 4-1.

The results show that MockT has the slowest proliferation rate, meso G1 CAR has a slower proliferation rate, meso G2 CAR has a faster proliferation rate, and meso G1 CAR-137DCR2 has the fastest proliferation rate.

### Example 4-(2): Detection of Cell Proliferation Activity by Cell Proliferation Activity Kit

1. Experimental Samples and Reagents

The recombinant cells Muc1 G1 CAR, Muc1 G2 CAR, Muc1 G1 CAR-28DCR2, 28DCR1, 28DCR2, 28DCR3 and Mock T prepared according to Example 2-(2).

CellTiter-Glo® Luminescent Cell Viability Assay, Promega, Cat. #G7570.

2. Experimental Method

(1) The above cells that have been cultured for 8 days were added in a 96-well white plate in 100 μL of AIM-V medium (containing cells) for each well.

(2) A blank control without cells was used to obtain the background fluorescence value.

(3) the complexes to be tested were added to the plate and incubated for 30 min in an incubator.

(4) 100 μL of CellTiter-Glo reagent was added, mixed well on an oscillator for 2 minutes, and incubated at room temperature for 10 minutes, then readings were obtained.

(5) Detections were made according to the above steps on the 8th, 9th, and 10th day of the culture.

3. Experimental Results

The results are shown in FIGS. 3-2 and 4-2.

FIG. 3-2 shows that MockT has the slowest proliferation rate, and 28DCR has a faster proliferation rate.

FIG. 4-2 shows that MockT has the slowest proliferation rate, Muc1 G1 CAR has a slower proliferation rate, Muc1 G2 CAR haw a faster proliferation rate, and Muc1 G1 CAR-28DCR2 has the fastest proliferation rate.

### Example 4-(3): Detection of Cell Proliferation Activity by Cell Proliferation Activity Kit

1. Experimental Samples and Reagents

The recombinant cells EGFR G1 CAR, EGFR G2 CAR, EGFR G1 CAR-40DCR2, 40DCR1, 40DCR2, 40DCR3 and Mock T prepared according to Example 2-(3).

CellTiter-Glo® Luminescent Cell Viability Assay, Promega, Cat. #G7570.

2. Experimental Method

(1) The above cells that have been cultured for 8 days were added in a 96-well white plate in 100 μL of AIM-V medium (containing cells) for each well.

(2) A blank control without cells was used to obtain the background fluorescence value.

(3) the complexes to be tested was added to the plate and incubated for 30 min in an incubator.

(4) 100 μL of CellTiter-Glo reagent was added, mixed well on an oscillator for 2 minutes, and incubated at room temperature for 10 minutes, then readings were obtained.

(5) Detections were made according to the above steps on the 8th, 9th, and 10th day of the culture.

3. Experimental Results

The results are shown in FIGS. 3-3 and 4-3.

FIG. 3-3 shows that MockT has the slowest proliferation rate, and 40DCR has a faster proliferation rate.

FIG. 4-3 shows that MockT has the slowest proliferation rate, EGFR G1 CAR has a slower proliferation rate, EGFR G2 CAR haw a faster proliferation rate, and EGFR G1 CAR-40DCR2 has the fastest proliferation rate.

### Example 5-(1): Detection of Phenotype of Dual Costimulatory Molecule Activated Receptor 137DCR Combined with Meso G1 CAR-T Cell by Flow Cytometry Under Stimulation with Mesothelin Antigen

1. Experimental Samples

The recombinant cells 137DCR1, 137DCR2, 137DCR 3, meso G1 CAR, meso G2 CAR, meso G1 CAR-137DCR3 and Mock T prepared according to Example 2-(1).

2. Experimental Method

The above cells were each collected and added to a 1.5 ml EP tube at 1×10⁶ cells/tube after counting, washed twice with PBS, centrifuged at 1200 rpm for 5 min, 2 μl of isotype control antibody IgG1-PE, fluorescence flow antibody anti-CD137, isotype control (IgG1FITC+IgG1PC5+IgG1PE), (anti-CD45RO-PC5, anti-CD62L-FITC, anti-CCR7-PE) were added, the precipitate was flicked to make it mix evenly, incubated at room temperature in dark for 30 min, washed with PBS once, 400 μl PBS was added, the cells were transferred to a flow tube, and detected on the machine.

3. Experimental Results

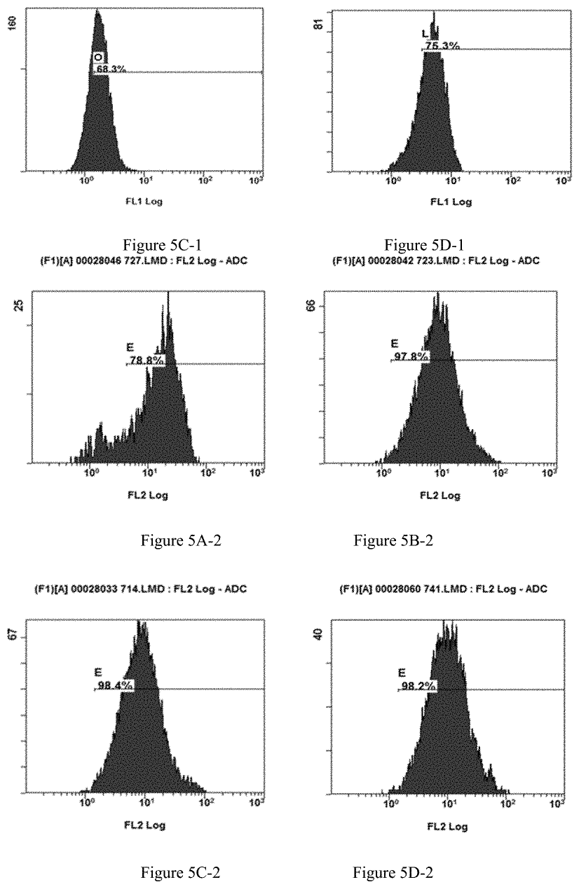

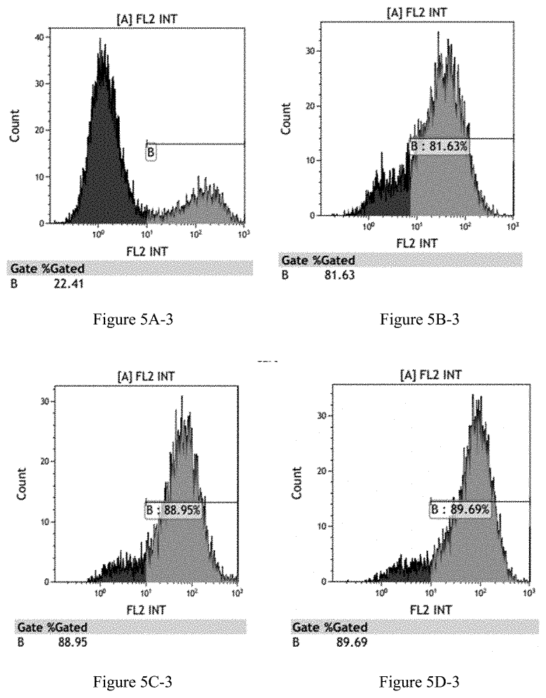

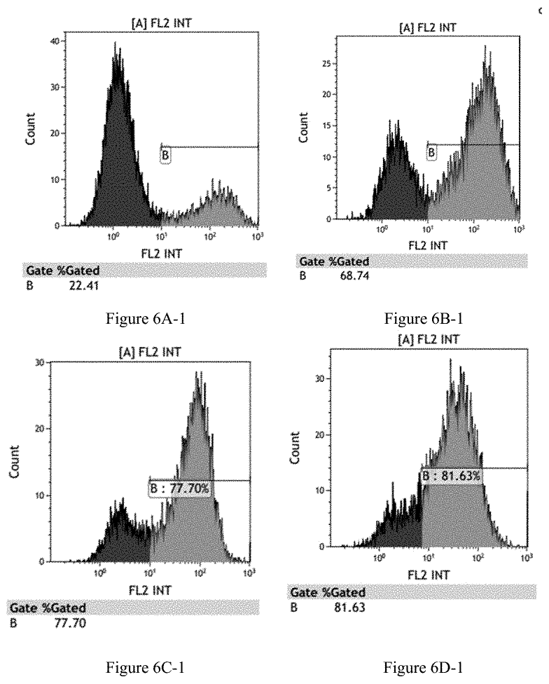

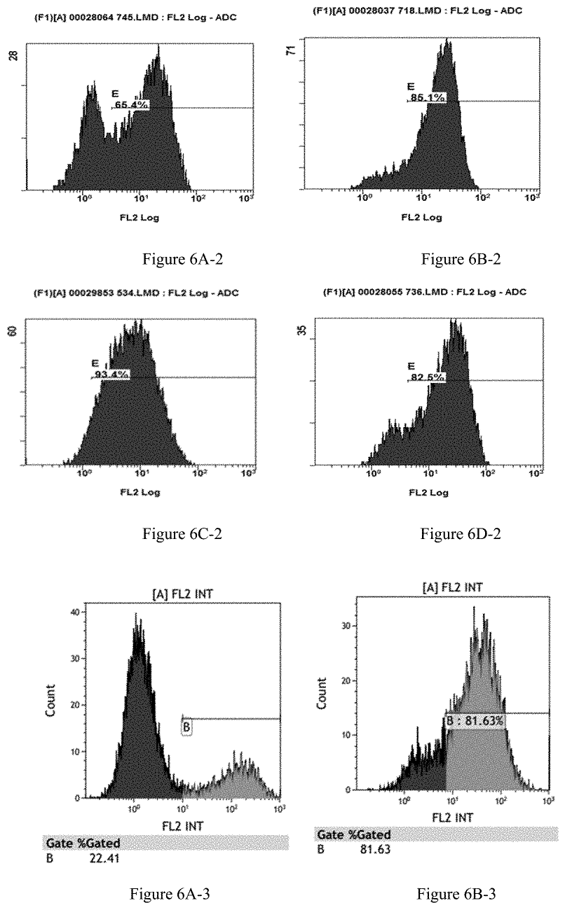

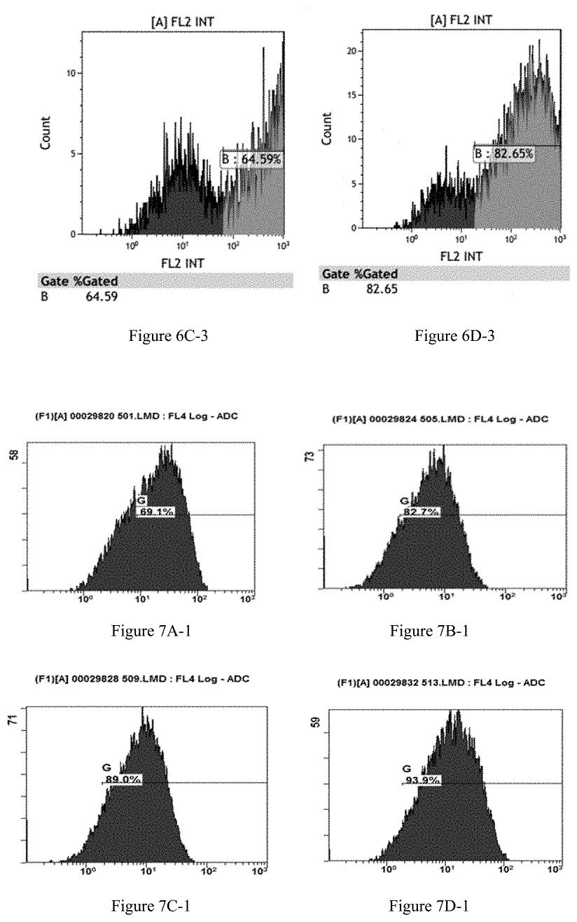

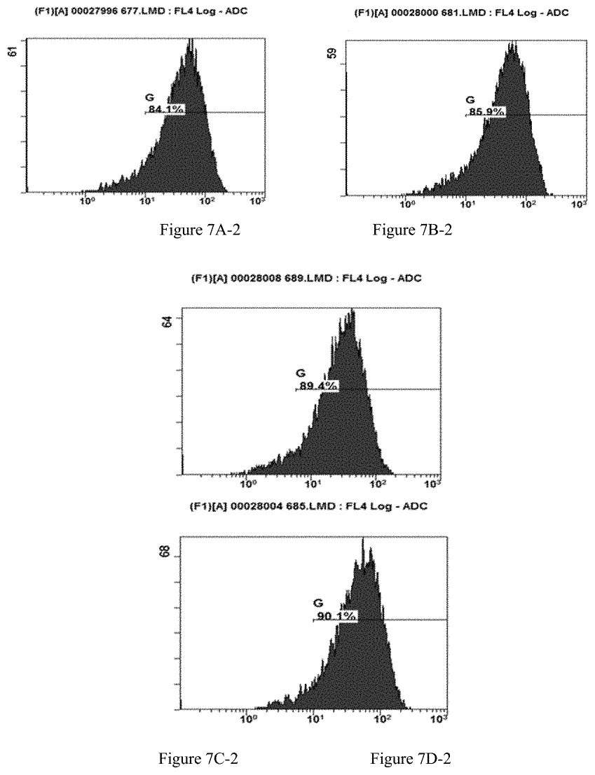

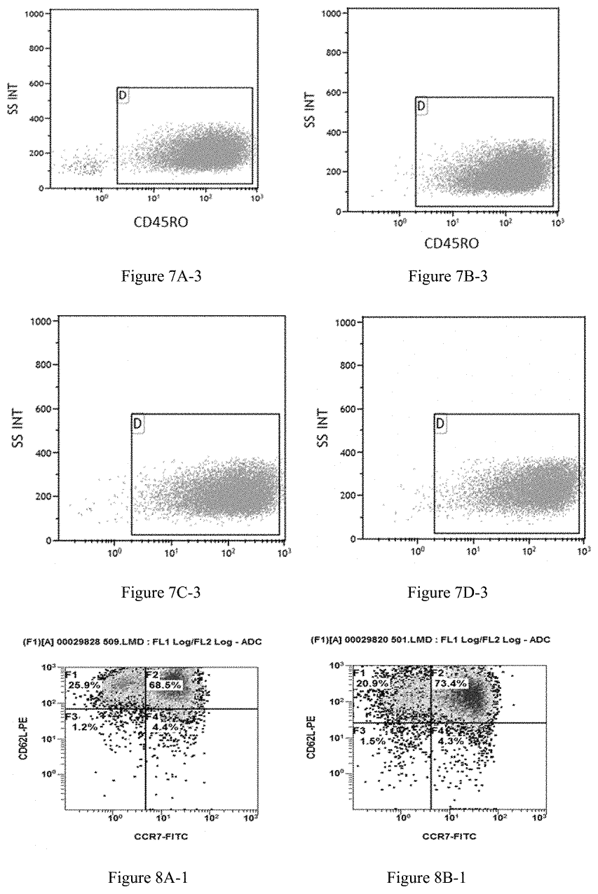

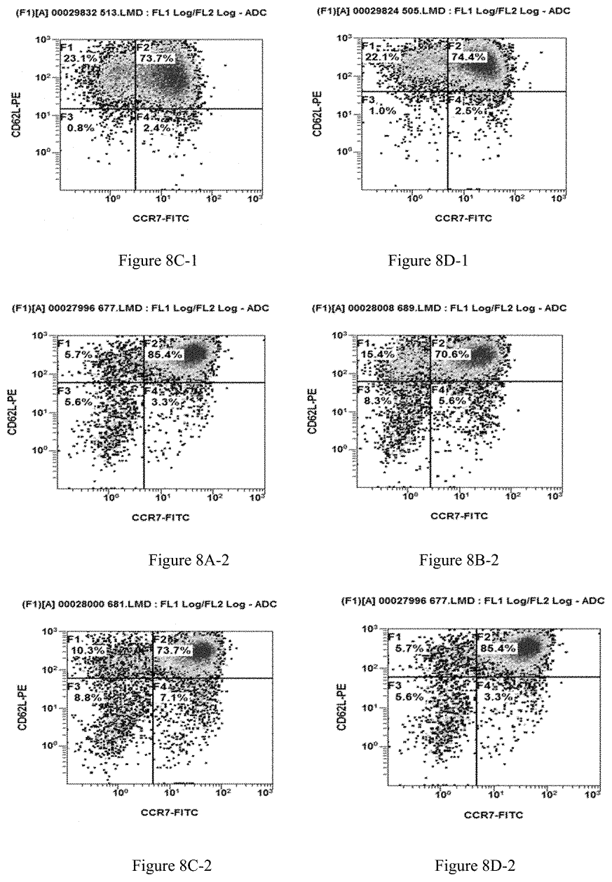

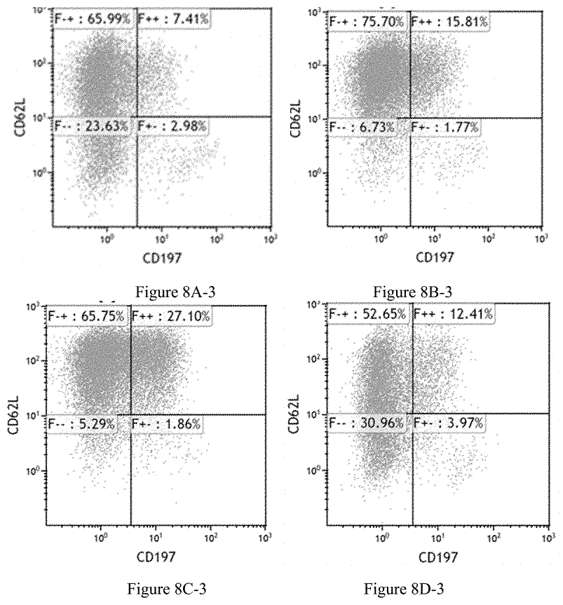

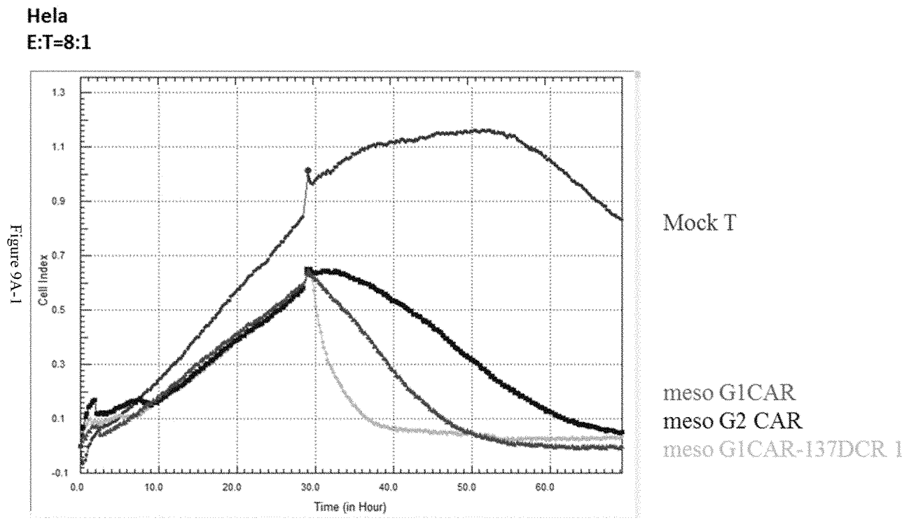

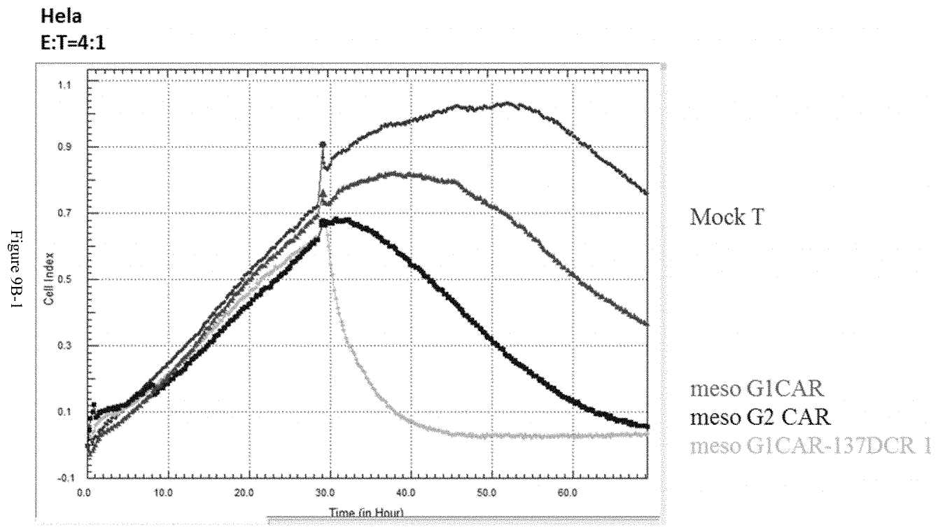

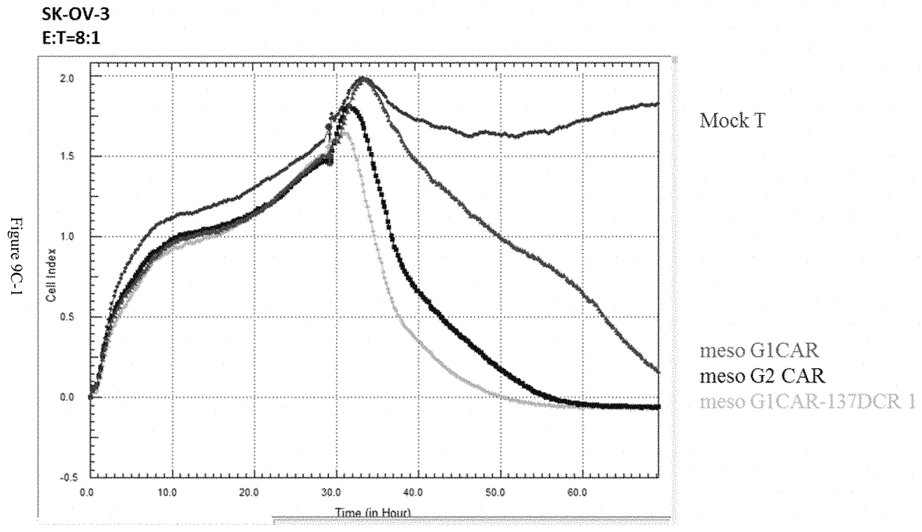

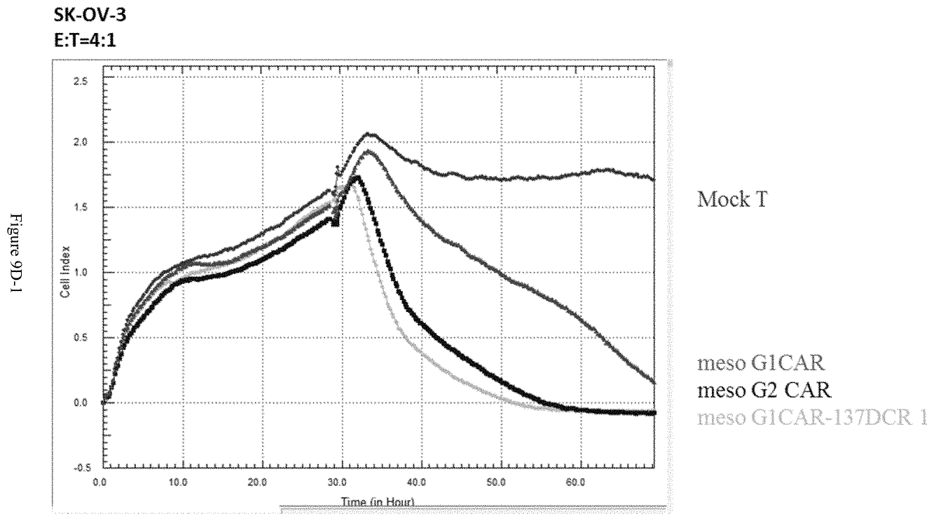

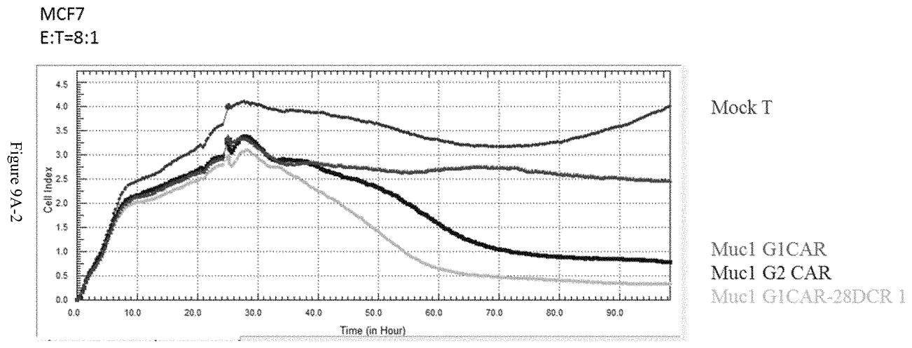

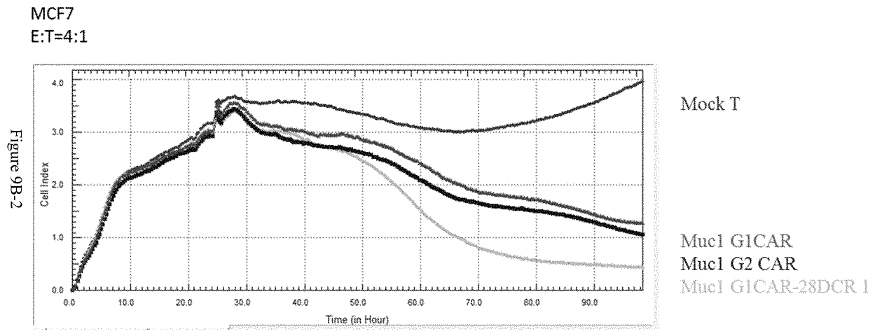

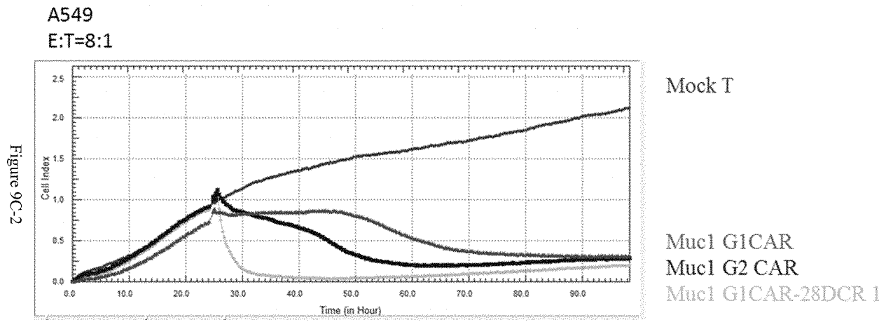

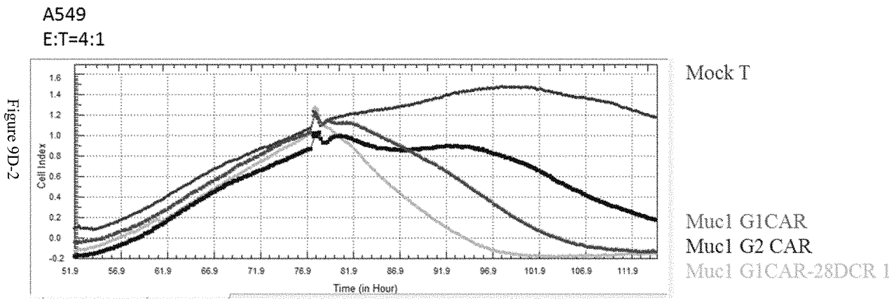

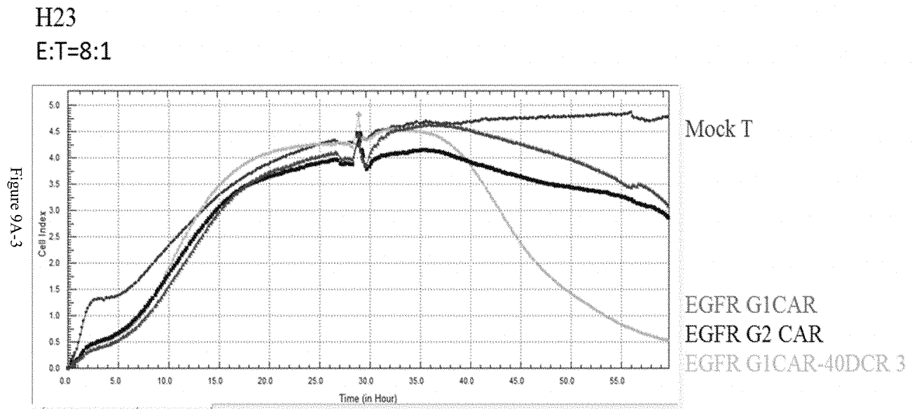

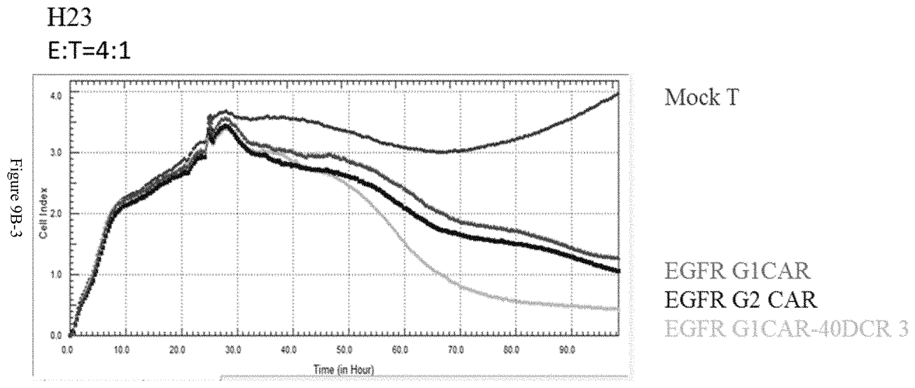

The results are shown in FIGS. 5A-1 to 5D-1, FIGS. 6A-1 to 6D-1, FIGS. 7A-1 to 7D-1 and FIGS. 8A-1 to 8D-1. Wherein:

FIGS. 5A-1 to 5D-1 show 3 single-transformed cells: 137DCR1, 137DCR2, and 137DCR3, with the CD137 phenotype greatly improved compared to Mock T.

FIGS. 6A-1 to 6D-1 show the CD137 phenotype of Mock T, meso G1 CAR, meso G2 CAR, and meso G1 CAR-137DCR2. Compared with the other three groups, meso G1 CAR-137DCR2 is greatly improved.

FIGS. 7A-1 to 7D-1 show the CD45RO phenotype of Mock T, meso G1 CAR, meso G2 CAR, meso G1 CAR-137DCR2, indicating the degree of cell activation. Cells have been activated in large amounts.

FIGS. 8A-1 to 8D-1 show the memory T phenotypes of Mock T, meso G1 CAR, meso G2 CAR, and meso G1 CAR-137DCR2. Compared with the other three groups, meso G1 CAR-137DCR2 promotes the formation of memory T.

### Example 5-(2): Detection of Phenotype of Dual Costimulatory Molecule Activated Receptor 28DCR Combined with Muc1 G1 CAR-T Cell by Flow Cytometry Under Stimulation with Muc1 Antigen

1. Experimental Samples

The recombinant cells Muc1 G1 CAR, Muc1 G2 CAR, Muc1 G1 CAR-28DCR3, 28DCR1, 28DCR2, 28DCR3 and Mock T prepared according to Example 2-(2).

2. Experimental Method

The above cells were each collected and added to a 1.5 ml EP tube at 1×10⁶ cells/tube after counting, washed twice with PBS, centrifuged at 1200 rpm for 5 min, 2 μl of isotype control antibody IgG1-PE, fluorescence flow antibody anti-CD137, isotype control (IgG1FITC+IgG1PC5+IgG1PE), (anti-CD45RO-PCS, anti-CD62L-FITC, anti-CCR7-PE) were added, the precipitate was flicked to make it mix evenly, incubated at room temperature in dark for 30 min, washed with PBS once, 400 μl PBS was added, the cells were transferred to a flow tube, and detected on the machine.

3. Experimental Results

The results are shown in FIGS. 5A-2 to 5D-2, FIGS. 6A-2 to 6D-2, FIGS. 7A-2 to 7D-2 and FIGS. 8A-2 to 8D-2. Wherein: FIGS. 5A-2 to 5D-2 show 3 single-transformed cells: 28DCR1, 28DCR2, and 28DCR3, with the CD137 phenotype greatly improved compared to Mock T.

FIGS. 6A-2 to 6D-2 show the CD137 phenotype of Mock T, Muc1 G1 CAR, Muc1 G2 CAR, and Muc1 G1 CAR-28DCR2. Compared with the other three groups, Muc1 G1 CAR-28DCR2 is greatly improved.

FIGS. 7A-2 to 7D-2 show the CD45RO phenotype of Mock T, Muc1 G1 CAR, Muc1 G2 CAR, Muc1 G1 CAR-28DCR2, indicating the degree of cell activation. Cells have been activated in large amounts.

FIGS. 8A-2 to 8D-2 show the memory T phenotype of Mock T, Muc1 G1 CAR, Muc1 G2 CAR, and Muc1 G1 CAR-28DCR2. Compared with the other three groups, Muc1 G1 CAR-28DCR2 promotes the formation of memory T.

### Example 5-(3): Detection of Phenotype of Dual Costimulatory Molecule Activated Receptor 40DCR Combined with EGFR G1 CAR-T Cell by Flow Cytometry Under Stimulation with EGFR Antigen

1. Experimental Samples

The recombinant cells EGFR G1 CAR, EGFR G2 CAR, EGFR G1 CAR-40DCR3, 40DCR1, 40DCR2, 40DCR3 and Mock T prepared according to Example 2-(3).

2. Experimental Method

The above cells were each collected and added to a 1.5 ml EP tube at 1×10⁶ cells/tube after counting, washed twice with PBS, centrifuged at 1200 rpm for 5 min, 2 μl of isotype control antibody IgG1-PE, fluorescence flow antibody anti-CD137, isotype control (IgG1FITC+IgG1PC5+IgG1PE), (anti-CD45RO-PCS, anti-CD62L-FITC, anti-CCR7-PE) were added, the precipitate was flicked to make it mix evenly, incubated at room temperature in dark for 30 min, washed with PBS once, 400 μl PBS was added, the cells were transferred to a flow tube, and detected on the machine.

3. Experimental Results

The results are shown in FIGS. 5A-3 to 5D-3, FIGS. 6A-3 to 6D-3, FIGS. 7A-3 to 7D-3 and FIGS. 8A-3 to 8D-3. Wherein: FIGS. 5A-3 to 5D-3 show 3 single-transformed cells 40DCR1, 40DCR2, and 40DCR3, with the CD137 phenotype greatly improved compared to Mock T;

FIGS. 6A-3 to 6D-3 show the CD137 phenotype of Mock T, EGFR G1 CAR, EGFR G2 CAR, and EGFR G1 CAR-40DCR2. Compared with the other three groups, EGFR G1 CAR-40DCR2 is greatly improved;

FIGS. 7A-3 to 7D-3 show the CD45RO phenotype of Mock T, EGFR G1 CAR, EGFR G2 CAR, EGFR G1 CAR-40DCR2, indicating the degree of cell activation. Cells have been activated in large amounts;

FIGS. 8A-3 to 8D-3 show the memory T phenotype of Mock T, EGFR G1 CAR, EGFR G2 CAR, and EGFR G1 CAR-40DCR2. Compared with the other three groups, EGFR G1 CAR-40DCR2 promotes the formation of memory T.

### Example 6-(1): Detection of the Killing Effect In Vitro of Dual Costimulatory Molecule Activated Receptor 137DCR Combined with Meso G1 CAR-T Cells on Tumor Cells by Real-Time Label-Free Cell Analysis System

1. Experimental Samples

Effector cells: the recombinant cells meso G1 CAR, meso G2 CAR, meso G1 CAR-137DCR1, meso G1 CAR-137DCR3 and Mock T prepared according to Example 2-(1).

Target cells: cervical cancer cell Hela, ovarian cancer cell SK-OV-3 (both from ATCC, American Type Culture Collection).

2. Experimental Method

Target cells and effector cells that matched in MHC class I typing were selected, and Real-time label-free cell analysis system (RTCA) was used to detect the killing effect of the above cells in vitro. The steps were as follows:

(1) Zero adjustment: 50 μl DMEM or 1640 culture medium was added to each well, put into the instrument, step 1 was selected for zero adjustment;

(2) Target cells plating: cervical cancer cell Hela and ovarian cancer cell SK-OV-3 were each plated at 10⁴ cells/50 μl per well on a plate containing detection electrodes, placed for a few minutes to stabilize the cells, then put into the instrument, step 2 was started to culture the cells;

(3) Adding effector cells: After 24 h culture of target cells, step 2 was paused and effector cells were added at 50 μl per well, with the effector target ratio of 8:1 or 4:1 (both 10⁴ tumor cells) and non-transferred Mock T cells were used as a control, step 3 was started to continue co-cultivation for 24 h, then the cell proliferation curve was observed.

2. Experimental Results

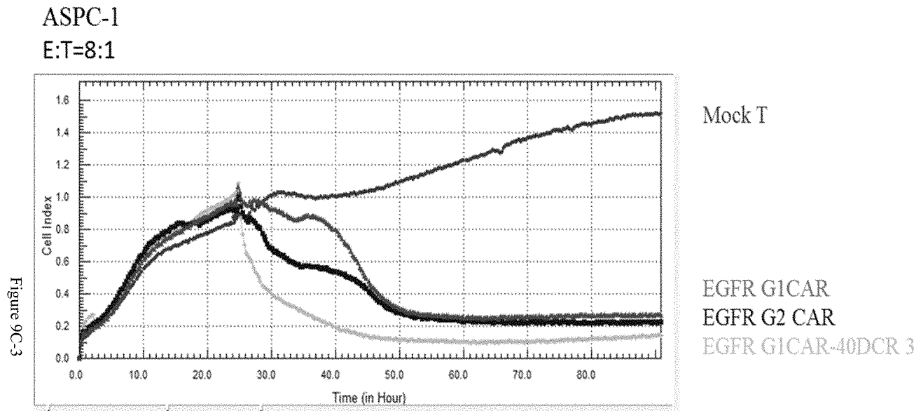

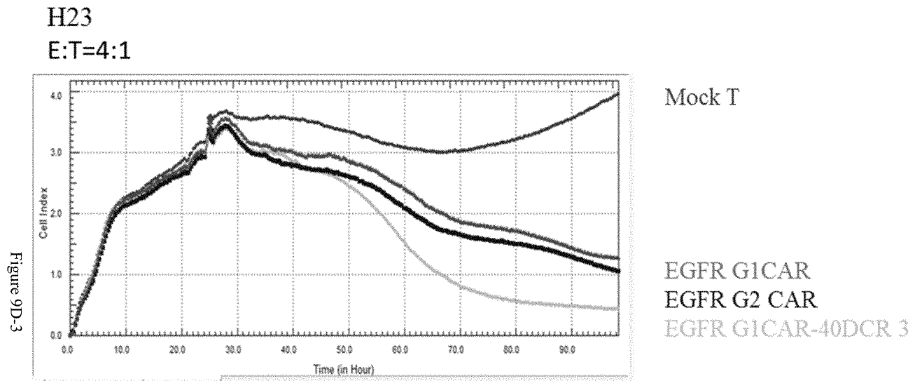

The results are shown in FIGS. 9A-1 to 9D-1.

The results show that Mock T has the weakest killing effect on tumor cells, meso G1 CAR has a weaker killing effect on tumor cells, meso G2 CAR has a stronger killing effect on tumor cells, meso G1 CAR-137DCR1 and meso G1 CAR-137DCR3 have the strongest killing effect on tumor cells.

### Example 6-(2): Detection of the Killing Effect In Vitro of Dual Costimulatory Molecule Activated Receptor 28DCR Combined with Muc1 G1 CAR-T Cells on Tumor Cells by Real-Time Label-Free Cell Analysis System

1. Experimental Samples

Effector cells: the recombinant cells Muc1 G1 CAR, Muc1 G2 CAR, Muc1 G1 CAR-28DCR1, Muc1 G1 CAR-28DCR3, 28DCR1, 28DCR2, 28DCR3 and Mock T prepared according to Example 2-(2).

Target cells: cervical cancer cell Hela, ovarian cancer cell SK-OV-3 (both from ATCC, American Type Culture Collection).

2. Experimental Method

Target cells and effector cells that matched in MHC class I typing were selected, and Real-time label-free cell analysis system (RTCA) was used to detect the killing effect of the above cells in vitro. The steps were as follows:

(1) Zero adjustment: 50 μl DMEM or 1640 culture medium was added to each well, put into the instrument, step 1 was selected for zero adjustment;

(2) Target cells plating: cervical cancer cell Hela and ovarian cancer cell SK-OV-3 were each plated at 10⁴ cells/50 μl per well on a plate containing detection electrodes, placed for a few minutes to stabilize the cells, then put into the instrument, step 2 was started to culture the cells;

(3) Adding effector cells: After 24 h culture of target cells, step 2 was paused and effector cells were added at 50 μl per well, with the effector target ratio of 8:1 or 4:1 (both 10⁴ tumor cells) and non-transferred Mock T cells were used as a control, step 3 was started to continue co-cultivation for 24 h, then the cell proliferation curve was observed.

2. Experimental Results

The results are shown in FIGS. 9A-2 to 9D-2.

The results show that Mock T has the weakest killing effect on tumor cells, Muc1 G1 CAR has a weaker killing effect on tumor cells, Muc1 G2 CAR has a stronger killing effect on tumor cells, Muc1 G1 CAR-28DCR1 and Muc1 G1 CAR-28DCR3 have the strongest killing effect on tumor cells.

### Example 6-(3): Detection of the Killing Effect In Vitro of Dual Costimulatory Molecule Activated Receptor 40DCR Combined with EGFR G1 CAR-T Cells on Tumor Cells by Real-Time Label-Free Cell Analysis System

1. Experimental Samples

Effector cells: the recombinant cells EGFR G1 CAR, EGFR G2 CAR, EGFR G1 CAR-40DCR1, EGFR G1 CAR-40DCR3, 40DCR1, 40DCR2, 40DCR3 and Mock T prepared according to Example 2-(3).

Target cells: cervical cancer cell Hela, ovarian cancer cell SK-OV-3 (both from ATCC, American Type Culture Collection).

2. Experimental Method

Target cells and effector cells that matched in MHC class I typing were selected, and Real-time label-free cell analysis system (RTCA) was used to detect the killing effect of the above cells in vitro. The steps were as follows:

(1) Zero adjustment: 50 μl DMEM or 1640 culture medium was added to each well, put into the instrument, step 1 was selected for zero adjustment;

(2) Target cell plating: cervical cancer cell Hela and ovarian cancer cell SK-OV-3 were each plated at 10⁴ cells/50 μl per well on a plate containing detection electrodes, placed for a few minutes to stabilize the cells, then put into the instrument, step 2 was started to culture the cells;

(3) Adding effector cells: After 24 h culture of target cells, step 2 was paused and effector cells were added at 50 μl per well, with the effector target ratio of 8:1 or 4:1 (both 10⁴ tumor cells) and non-transferred Mock T cells were used as a control, step 3 was started to continue co-cultivation for 24 h, then the cell proliferation curve was observed.

2. Experimental Results

The results are shown in FIGS. 9A-3 to 9D-3.

The results show that Mock T has the weakest killing effect on tumor cells, EGFR G1 CAR has a weaker killing effect on tumor cells, EGFR G2 CAR has a stronger killing effect on tumor cells, EGFR G1 CAR-40DCR1 and EGFR G1 CAR-40DCR3 have the strongest killing effect on tumor cells.

### Example 7-(1): Detection by Flow Cytometry of Cytokines Secretion of Meso-CAR-T Upon Stimulation with Mesothelin Antigen

1. Experimental Samples

The recombinant cells meso G1 CAR, meso G2 CAR, meso G1 CAR-137DCR1 and Mock T prepared according to Example 2-(1).

2. Experimental Method

1. A 96-well plate was coated with 5 μg/ml mesothelin antigen overnight at 4° C., washed 3 times with PBS, and added with 1×10⁵ cells of each sample separately, and the cell supernatant was collected after 24 hours of culture. BD™ CBA Human Th1/Th2 Cytokine Kit II was used to detect the cytokines secretion of meso CAR-T cells upon stimulation with mesothelin antigen.

Steps were as follows:

(1) Human IL-2, IL-4, IL-6, IL-10, TNF, IFN-γ capture magnetic beads were mixed by vortex, 50 μl of mixed beads was added to each tube;

(2) 50 μl of human Th1/Th2 cytokine standard (doubling diluted to 5000 pg/ml, 2500 pg/ml, 1250 pg/ml, 625 pg/ml, 312.5 pg/ml, 156 pg/ml, 80 pg/ml, 40 pg/ml, 20 pg/ml, or 0 pg/ml) and 50 μl of the sample to be tested (diluted by 2-fold with the diluent) were added;

(3) 50 μl of human Th1/Th2-II-PE detection antibody was added to each tube;

(4) Incubated at room temperature in dark for 3 h;

(5) 1 ml of Wash Buffer was added to each tube, centrifuged at 200 for 5 min, and the supernatant was discarded;

(6) 300 μl of Wash Buffer was added to each tube to resuspend the cells, and transferred to a flow cytometry tube for detecting the fluorescence value by a flow cytometer.

3. Experimental Results

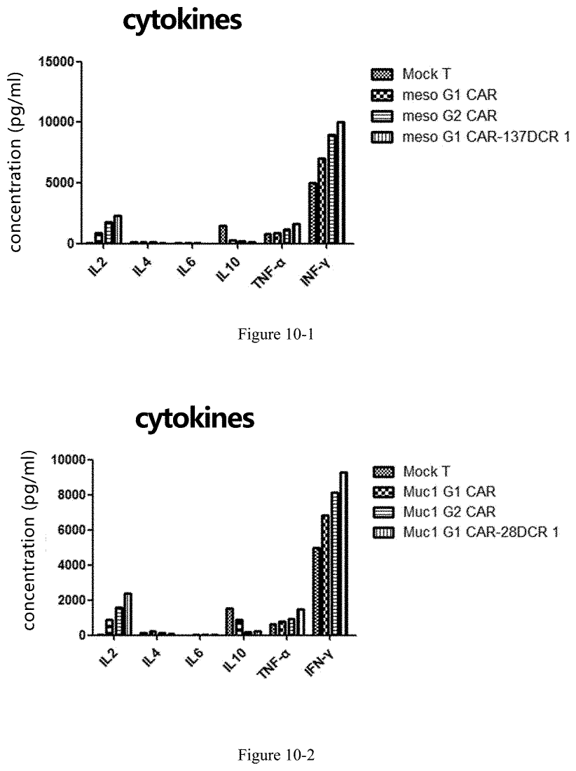

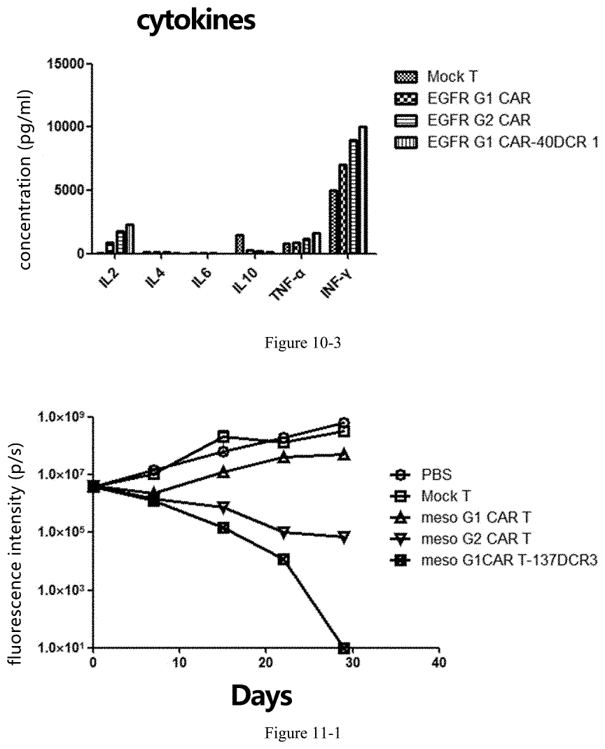

The results are shown in FIG. 10-1.

The results show that compared with Mock T cells, the secretion amounts of various cytokines (IL-2, IL-4, IL-6, IL-10, TNF-α and IFN-γ) by other cells are greatly improved. Mock T has the lowest secretion amounts of various cytokines, meso G1 CAR has lower secretion amounts of various cytokines, meso G2 CAR has higher secretion amounts of various cytokines, meso G1 CAR-137DCR1 has the highest secretion amounts of various cytokines.

### Example 7-(2): Detection by Flow Cytometry of Secretion Amounts of Cytokines by Muc1-CAR-T Upon Stimulation with Muc1 Antigen

1. Experimental Samples

The recombinant cells Muc1 G1 CAR, Muc1 G2 CAR, Muc1 G1 CAR-28DCR1 and Mock T prepared according to Example 2-(2).

2. Experimental Method

1. A 96-well plate was coated with 5 μg/ml Muc1 antigen overnight at 4° C., washed 3 times with PBS, and added with 1×10⁵ cells of each sample separately, and the cell supernatant was collected after 24 hours of culture. BD™ CBA Human Th1/Th2 Cytokine Kit II was used to detect the cytokines secretion of Muc1 CAR-T cells upon stimulation with Muc1 antigen.

Steps are as follows:

(1) Human IL-2, IL-4, IL-6, IL-10, TNF, IFN-γ capture magnetic beads were mixed by vortex, 50 μl of mixed beads was added to each tube;

(2) 50 μl of human Th1/Th2 cytokine standard (doubling diluted to 5000 pg/ml, 2500 pg/ml, 1250 pg/ml, 625 pg/ml, 312.5 pg/ml, 156 pg/ml, 80 pg/ml, 40 pg/ml, 20 pg/ml, or 0 pg/ml) and 50 μl of the sample to be tested (diluted by 2-fold with the diluent) were added;

(3) 50 μl of human Th1/Th2-II-PE detection antibody was added to each tube;

(4) Incubated at room temperature in dark for 3 h;

(5) 1 ml of Wash Buffer was added to each tube, centrifuged at 200 for 5 min, and the supernatant was discarded;

(6) 300 μl of Wash Buffer was added to each tube to resuspend the cells, and transferred to a flow cytometry tube for detecting the fluorescence value by a flow cytometer.

3. Experimental Results

The results are shown in FIG. 10-2.

The results show that compared with Mock T cells, the secretion amounts of various cytokines (IL-2, IL-4, IL-6, IL-10, TNF-α and IFN-γ) by other cells are greatly improved. Mock T has the lowest secretion amounts of various cytokines, Muc1 G1 CAR has lower secretion amounts of various cytokines, Muc1 G2 CAR has higher secretion amounts of various cytokines, Muc1 G1 CAR-28DCR1 has the highest secretion amounts of various cytokines.

### Example 7-(3): Detection by Flow Cytometry of Secretion Amounts of Cytokines by EGFR-CAR-T Upon Stimulation with EGFR Antigen

1. Experimental Samples

The recombinant cells EGFR G1 CAR, EGFR G2 CAR, EGFR G1 CAR-40DCR1 and Mock T prepared according to Example 2-(3).

2. Experimental Method

1. A 96-well plate was coated with 5 μg/ml EGFR antigen overnight at 4° C., washed 3 times with PBS, and added with 1×10⁵ cells of each sample separately, and the cell supernatant was collected after 24 hours of culture. BD′CBA Human Th1/Th2 Cytokine Kit II was used to detect the cytokines secretion of EGFR CAR-T cells upon stimulation with EGFR antigen.

Steps are as follows:

(1) Human IL-2, IL-4, IL-6, IL-10, TNF, IFN-γ capture magnetic beads were mixed by vortex, 50 μl of mixed beads was added to each tube;

(2) 50 μl of human Th1/Th2 cytokine standard (doubling diluted to 5000 pg/ml, 2500 pg/ml, 1250 pg/ml, 625 pg/ml, 312.5 pg/ml, 156 pg/ml, 80 pg/ml, 40 pg/ml, 20 pg/ml, or 0 pg/ml) and 50 μl of the sample to be tested (diluted by 2-fold with the diluent) were added;

(3) 50 μl of human Th1/Th2-II-PE detection antibody was added to each tube;

(4) Incubated at room temperature in dark for 3 h;

(5) 1 ml of Wash Buffer was added to each tube, centrifuged at 200 for 5 min, and the supernatant was discarded;

(6) 300 μl of Wash Buffer was added to each tube to resuspend the cells, and transferred to a flow cytometry tube for detecting the fluorescence value by a flow cytometer.

3. Experimental Results

The results are shown in FIG. 10-3.

The results show that compared with Mock T cells, the secretion amounts of various cytokines (IL-2, IL-4, IL-6, IL-10, TNF-α and IFN-γ) by other cells are greatly improved. Mock T has the lowest secretion amounts of various cytokines, EGFR G1 CAR has lower secretion amounts of various cytokines, EGFR G2 CAR has higher secretion amounts of various cytokines, EGFR G1 CAR-40DCR1 has the highest secretion amounts of various cytokines.

### Example 8-(1): Functional Assay In Vivo of Dual Costimulatory Molecule Activated Receptor 137 DCR Combined with Meso G1 CAR-T Cells

1. Experimental Samples and Animals

20 NSG immunodeficiency mice of 4-6 weeks old, with an average weight of 22-27 g, were provided by Beijing Vitalstar Biotech Co., Ltd., and raised by a SPF animal laboratory.

The recombinant cells meso G1 CAR, meso G2 CAR, meso G1 CAR-137DCR3 and Mock T prepared according to Example 2-(1).

Ovarian cancer cell SK-OV-3-luc (Shanghai Huiying Biotechnology Co., Ltd.).

2. Experimental Method

(1) Adhered human ovarian cancer cells SK-OV-3-luc in logarithmic growth phase cultured in vitro were digested with 0.25% trypsin, centrifuged, collected and re-suspended in PBS solution. The cells were centrifuged at 1000 rmp for 2 minutes at room temperature, the supernatant was discarded and the cells were suspended in PBS solution, centrifuged to collect the cells, and the cell concentration of the cell suspension was adjusted to 5×10⁷ cells/ml.

(2) The SK-OV-3-luc cells were inoculated subcutaneously in the right dorsum of the mouse at 0.1 ml/mouse. 10 days after the inoculation, the sizes of the tumors were observed by a living body imager.

(4) the NSG immunodeficient mice were randomly divided into 4 groups (five mice each group). The administration was conducted through the tail vein injection at 0.1 ml per mouse (5×10⁶ positive cells) with PBS as the solvent. The administration was conducted only once.

(4) the living states of mice were observed every day and the change of the tumor in each mouse was observed by a living body imager every 10 days.

3. Experimental Results

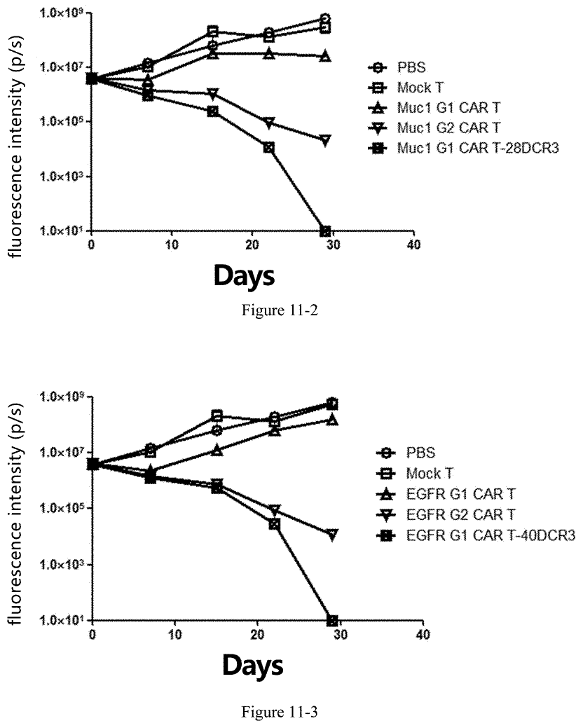

The results are shown in FIG. 11-1.

The results show the following: for the control group Mock T, the tumor cells have a strong fluorescence intensity; for the first generation mesothelin-targeting CAR, meso G1 CAR group, the tumor cells have a weakened fluorescence intensity, showing a certain therapeutic effect; for meso G2 CAR, the tumor cells have a weaker fluorescence intensity, showing a better therapeutic effect; for meso G1 CAR-137DCR3, the tumor cells have the weakest fluorescence intensity, showing the best therapeutic effect.

### Example 8-(2): Functional Experiment In Vivo of Dual Costimulatory Molecule Activated Receptor 28DCR Combined with Muc1 G1 CAR-T Cells

1. Experimental Samples and Animals

20 NSG immunodeficiency mice of 4-6 weeks old, with an average weight of 22-27 g, were provided by Beijing Vitalstar Biotech Co., Ltd., and raised by a SPF animal laboratory.

The recombinant cells Muc1 G1 CAR, Muc1 G2 CAR, Muc1 G1 CAR-28DCR3 and Mock T prepared according to Example 2-(2).

Ovarian cancer cell SK-OV-3-luc (Shanghai Huiying Biotechnology Co., Ltd.).

2. Experimental Method

(1) Adhered human ovarian cancer cells SK-OV-3-luc in logarithmic growth phase cultured in vitro were digested with 0.25% trypsin, centrifuged, collected and re-suspended in PBS solution. The cells were centrifuged at 1000 rmp for 2 minutes at room temperature, the supernatant was discarded and the cells were suspended in PBS solution, centrifuged to collect the cells, and the cell concentration of the cell suspension was adjusted to 5×10⁷ cells/ml.

(2) The SK-OV-3-luc cells were inoculated subcutaneously in the right dorsum of the mouse at 0.1 ml/mouse. 10 days after the inoculation, the sizes of the tumors were observed by a living body imager.

(3) the NSG immunodeficient mice were randomly divided into 4 groups (five mice each group). The administration was conducted through the tail vein injection at 0.1 ml per mouse (5×10⁶ positive cells) with PBS as the solvent. The administration was conducted only once.

(4) the living states of mice were observed every day and the change of the tumor in each mouse was observed by the living body imager every 10 days.

3. Experimental Results

The results are shown in FIG. 11-2.

The results show the following: for the control group Mock T, the tumor cells have strong fluorescence intensity; for the first generation Muc1-targeting CAR, Muc1 G1 CAR, the tumor cells have a weakened fluorescence intensity, showing a certain therapeutic effect; for Muc1 G2 CAR, the tumor cells have a weaker fluorescence intensity, showing a better therapeutic effect; for Muc1 G1 CAR-28DCR3, the tumor cells have the weakest fluorescence intensity, showing the best therapeutic effect.

### Example 8-(3): Functional Experiment In Vivo of Dual Costimulatory Molecule Activated Receptor 40DCR Combined with EGFR G1 CAR-T Cells 1. Experimental Samples and Animals

20 NSG immunodeficiency mice of 4-6 weeks old, with an average weight of 22-27 g, were provided by Beijing Vitalstar Biotech Co., Ltd., and raised by a SPF animal laboratory.

The recombinant cells EGFR G1 CAR, EGFR G2 CAR, EGFR G1 CAR-40DCR3 and Mock T prepared according to Example 2-(3).

Ovarian cancer cell SK-OV-3-luc (Shanghai Huiying Biotechnology Co., Ltd.).

2. Experimental Method

(1) Adhered human ovarian cancer cells SK-OV-3-luc in logarithmic growth phase cultured in vitro were digested with 0.25% trypsin, centrifuged, collected and re-suspended in PBS solution. The cells were centrifuged at 1000 rmp for 2 minutes at room temperature, the supernatant was discarded and the cells were suspended in PBS solution, centrifuged to collect the cells, and the cell concentration of the cell suspension was adjusted to 5×10⁷ cells/ml.

(2) The SK-OV-3-luc cells were inoculated subcutaneously in the right dorsum of the mouse at 0.1 ml/mouse. 10 days after the inoculation, the sizes of the tumors were observed by a living body imager.

(3) the NSG immunodeficient mice were randomly divided into 4 groups (five mice each group). The administration was conducted through the tail vein injection at 0.1 ml per mouse (5×10⁶ positive cells) with PBS as the solvent. The administration was conducted only once.

(4) the living states of mice were observed every day and the change of the tumor in each mouse was observed by the living body imager every 10 days.

3. Experimental Results

The results are shown in FIG. 11-3.

The results show the following: for the control group Mock T, the tumor cells have a strong fluorescence intensity; for the first generation EGFR-targeting CAR, EGFR G1 CAR, the tumor cells have a weakened fluorescence intensity, showing a certain therapeutic effect; for EGFR G2 CAR, the tumor cells have a weaker fluorescence intensity, showing a better therapeutic effect; for EGFR G1 CAR-28DCR3, the tumor cells have the weakest fluorescence intensity, showing the best therapeutic effect.

Although specific embodiments of the present disclosure have been described in detail, those skilled in the art will understand, based on the disclosed teachings, various modifications and substitutions can be made to those details, and these changes are all within the protection scope of the present disclosure. The scope of the invention is provided by the appended claims and any equivalents thereof.

1-20. (canceled)

21. An isolated polypeptide, comprising, from N-terminus to C-terminus, the following elements:
an optional signal peptide, a polypeptide that activates a costimulatory signal molecule, an extracellular hinge region, a transmembrane region, and an intracellular costimulatory signal molecule.

22. The polypeptide according to claim 21, wherein the polypeptide that activates a costimulatory signal molecule is an agonistic single-chain antibody of the costimulatory signal molecule or a ligand of the costimulatory signal molecule.

23. The polypeptide according to claim 21, wherein the polypeptide is characterized by any 1, 2, 3 or 4 of the following items (1) to (4):
(1) the signal peptide is a membrane protein signal peptide selected from the group consisting of a CD8 signal peptide, a CD28 signal peptide, and a CD4 signal peptide;
(2) the extracellular hinge region is one or more members selected from the group consisting of an IgG4Fc CH2CH3 hinge region, a CD28 hinge region and a CD8 hinge region;
(3) the transmembrane region is one or more members selected from the group consisting of a CD28 transmembrane region, a CD8 transmembrane region, a CD3 transmembrane region, a CD134 transmembrane region, a CD137 transmembrane region, an ICOS transmembrane region and a DAP10 transmembrane region; and
(4) the intracellular costimulatory signal molecule is any one or more of members selected from the group consisting of a CD28 intracellular domain, a CD134/OX40 intracellular domain, a CD137/4-1BB intracellular domain, an LCK intracellular domain, an ICOS intracellular domain and a DAP10 intracellular domain.

24. The isolated polypeptide according to claim 21, wherein:
the signal peptide is a CD8 signal peptide as shown in SEQ ID NO: 1;
the extracellular hinge region is a CD8 hinge region as shown in SEQ ID NO: 3, or a IgG4Fc CH2CH3 hinge region as shown in SEQ ID NO: 56;
the transmembrane region is a CD28 transmembrane region as shown in SEQ ID NO: 4;
the intracellular costimulatory signal molecule is a CD28 intracellular domain as shown in SEQ ID NO: 5, or a CD137 intracellular domain as shown in SEQ ID NO: 6.

25. The isolated polypeptide according to claim 22, wherein:
the agonistic single-chain antibody of the costimulatory signal molecule is any one or more members selected from the group consisting of a CD137 agonistic single-chain antibody, a CD28 agonistic single-chain antibody and a CD40 agonistic single-chain antibody;
the ligand of the costimulatory signal molecule is any one or more members selected from the group consisting of a ligand of CD137, a ligand of CD28 and a ligand of CD40.

26. The isolated polypeptide according to claim 25, wherein:
the amino acid sequence of the CD137 agonistic single-chain antibody is shown in SEQ ID NO: 2;
the amino acid sequence of the CD28 agonistic single-chain antibody is shown in SEQ ID NO: 31;
the amino acid sequence of the CD40 agonistic single-chain antibody is as shown in SEQ ID NO: 55;
the ligand of CD137 is 4-1BBL;
the ligand of CD28 is CD80/CD86;
the ligand of CD40 is CD40L.

27. The polypeptide according to claim 21, comprising, from N-terminus to C-terminus, the following elements:
an optional CD8 signal peptide, a CD137 agonistic single-chain antibody, a CD8 extracellular hinge region, a CD28 transmembrane region, and a CD28 intracellular domain and/or a CD137 intracellular domain;
an optional CD8 signal peptide, a CD28 agonistic single-chain antibody, a CD8 extracellular hinge region, a CD28 transmembrane region, and a CD28 intracellular domain and/or a CD137 intracellular domain; or
an optional CD8 signal peptide, a CD40 agonistic single-chain antibody, a IgG4Fc CH2CH3 hinge region, a CD28 transmembrane region, and a CD28 intracellular domain and/or a CD137 intracellular domain.

28. The polypeptide according to claim 21, wherein the amino acid sequence of the isolated polypeptide is as shown in any one of SEQ ID NOs: 7 to 14;
any one of SEQ ID NOs: 32 to 39; or
any one of SEQ ID NOs: 57 to 64.

29. An isolated polynucleotide, wherein the isolated polynucleotide encodes the isolated polypeptide according to claim 21.

30. The isolated polynucleotide according to claim 29, wherein the sequence of the isolated polynucleotide is as shown in any one of SEQ ID NO: 15 or 22;
any one of SEQ ID NOs: 40 to 47; or
any one of SEQ ID NOs: 65 to 72.

31. A nucleic acid construct comprising the isolated polynucleotide according to claim 29.

32. The nucleic acid construct according to claim 31, wherein the sequence of the isolated polynucleotide is as shown in any one of SEQ ID NO: 15 or 22; any one of SEQ ID NOs:
40 to 47; or any one of SEQ ID NOs: 65 to 72.

33. A recombinant vector, wherein the recombinant vector comprises the isolated polynucleotide according to claim 29 or a nucleic acid construct comprising the polynucleotide.

34. A combination of recombinant vectors, comprising a first recombinant vector and a second recombinant vector, wherein:
the first recombinant vector comprises the isolated polynucleotide according to claim 29 or a nucleic acid construct comprising the polynucleotide,
the second recombinant vector contains a coding sequence of a first-generation chimeric antigen receptor.

35. The combination of recombinant vectors according to claim 34, wherein the first-generation chimeric antigen receptor is a first-generation chimeric antigen receptor that targets mesothelin, Mud 1 or EGFR.

36. The combination of recombinant vectors according to claim 35, wherein the amino acid sequence of the first-generation chimeric antigen receptor is as shown in SEQ ID NO: 23, SEQ ID NO: 48 or SEQ ID NO: 73, or the nucleic acid sequence of the first-generation chimeric antigen receptor is as shown in SEQ ID NO: 24, SEQ ID NO: 49 or SEQ ID NO: 74.

37. The combination of recombinant vectors according to claim 35, wherein the second recombinant vector is a recombinant PNB328 vector.

38. A recombinant host cell, wherein the cell contains the polynucleotide according to claim 25, or a nucleic acid construct comprising the polynucleotide, a recombinant vector comprising the polynucleotide or the nucleic acid construct, or a combination of recombinant vectors comprising a first recombinant vector and a second recombinant vector, wherein the first recombinant vector comprises the polynucleotide or the nucleic acid construct and the second recombinant vector contains a coding sequence of a first-generation chimeric antigen receptor.

39. A T cell, wherein the T cell expresses the polypeptide according to claim 21 and a first-generation chimeric antigen receptor.

40. The T cell according to claim 39, wherein the recombinant T cell is a recombinant peripheral blood mononuclear cell.

41. The T cell according to claim 39, wherein the first-generation chimeric antigen receptor is a first-generation chimeric antigen receptor that targets mesothelin, Muc1 or EGFR.

42. The T cell according to claim 39, wherein the amino acid sequence of the first-generation chimeric antigen receptor is as shown in SEQ ID NO: 23, SEQ ID NO: 48 or SEQ ID NO: 73.

43. A pharmaceutical composition comprising the polypeptide according to claim 21, a polynucleotide encoding the polypeptide, or a nucleic acid construct containing the polynucleotide, or a recombinant vector comprising the polynucleotide or the nucleic acid construct, a combination of recombinant vectors comprising a first recombinant vector and a second recombinant vector, wherein the first recombinant vector comprises the polynucleotide or the nucleic acid construct and the second recombinant vector contains a coding sequence of a first-generation chimeric antigen receptor, a recombinant host cell comprising the polynucleotide or the nucleic acid construct or the recombinant vector or the combination of recombinant vectors, or T cells expressing the polypeptide and the first-generation chimeric antigen receptor; optionally, further comprising a pharmaceutically acceptable excipient.

44. A method of treating and/or preventing cancer, or inhibiting a cancer cell in vivo or in vitro, or promoting the secretion of a cytokine by T cells in vivo or in vitro, comprising the step of administering to a subject in need thereof, or the cancer cell, or the T cell, an effective amount of the polypeptide according to claim 21, or a polynucleotide encoding the polypeptide, or a nucleic acid construct containing the polynucleotide, or a recombinant vector comprising the polynucleotide or the nucleic acid construct, a combination of recombinant vectors comprising a first recombinant vector and a second recombinant vector, wherein the first recombinant vector comprising the polynucleotide or the nucleic acid construct and the second recombinant vector contains a coding sequence of a first-generation chimeric antigen receptor, a recombinant host cell comprising the polynucleotide or the nucleic acid construct or the recombinant vector or the combination of recombinant vectors, or T cells expressing the polypeptide and the first-generation chimeric antigen receptor.

45. The method according to claim 44, wherein the cancer is a cancer that abnormally expresses mesothelin, Muc1 or EGFR on the surface of its cancer cells; the cytokine is one or more members selected from the group consisting of IL-2, IL-4, IL-6, IL-10, TNF-α and IFN-γ.

46. The method according to claim 45, wherein the cancer is selected from the group consisting of adenocarcinoma, lung cancer, colon cancer, colorectal cancer, breast cancer, ovarian cancer, cervical cancer, gastric cancer, bile duct cancer, gallbladder cancer, esophageal cancer, pancreatic cancer or prostate cancer.


SEQUENCE LISTING (78 sequences)

SEQ ID NO: 1 - protein, 23 aa, Artificial Sequence
  Amino acid sequence of CD8 signal peptide
MGNSCYNIVATLLLVLNFERTRS

SEQ ID NO: 2 - protein, 246 aa, Artificial Sequence
  Amino acid sequence of CD137 extracellular agonistic single-chain antibody
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKE

SEQ ID NO: 3 - protein, 49 aa, Artificial Sequence
  Amino acid sequence of CD8alpha hinge region
SKYGPPCPPCPIEVMYPPPYLDNEKSNGTIIHVKGKHLCPSPLFPGPSK

SEQ ID NO: 4 - protein, 28 aa, Artificial Sequence
  Amino acid sequence of CD28 transmembrane region
PFWVLVVVGGVLACYSLLVTVAFIIFWV

SEQ ID NO: 5 - protein, 41 aa, Artificial Sequence
  Amino acid sequence of CD28 intracellular costimulatory signal domain
RSKRSRLLHSDYMNMTPRRPGPTRKHYQPYAPPRDFAAYRS

SEQ ID NO: 6 - protein, 42 aa, Artificial Sequence
  Amino acid sequence of CD137 intracellular costimulatory signal domain
KRGRKKLLYIFKQPFMRPVQTTQEEDGCSCRFPEEEEGGCEL

SEQ ID NO: 7 - protein, 387 aa, Artificial Sequence
  Amino acid sequence of 137DCR1 (including signal peptide)
MGNSCYNIVATLLLVLNFERTRSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 388 aa, Artificial Sequence
  Amino acid sequence of 137DCR2 (including signal peptide)
MGNSCYNIVATLLLVLNFERTRSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 9 - protein, 429 aa, Artificial Sequence
  Amino acid sequence of 137DCR3 (including signal peptide)
MGNSCYNIVATLLLVLNFERTRSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 10 - protein, 429 aa, Artificial Sequence
  Amino acid sequence of 137DCR4 (including signal peptide)
MGNSCYNIVATLLLVLNFERTRSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 11 - protein, 364 aa, Artificial Sequence
  Amino acid sequence of 137DCR1 (without signal peptide)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - protein, 365 aa, Artificial Sequence
  Amino acid sequence of 137DCR2 (without signal peptide)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 13 - protein, 406 aa, Artificial Sequence
  Amino acid sequence of 137DCR3 (without signal peptide)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 14 - protein, 406 aa, Artificial Sequence
  Amino acid sequence of 137DCR4 (without signal peptide)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 15 - DNA, 1167 bp, Artificial Sequence
  Nucleic acid sequence of 137DCR1 (including signal peptide)
atgggaaacagctgttacaacatagtagccactctgttgctggtcctcaactttgagaggacaagatcacaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 16 - DNA, 1170 bp, Artificial Sequence
  Nucleic acid sequence of 137DCR2 (including signal peptide)
atgggaaacagctgttacaacatagtagccactctgttgctggtcctcaactttgagaggacaagatcacaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 17 - DNA, 1293 bp, Artificial Sequence
  Nucleic acid sequence of 137DCR3 (including signal peptide)
atgggaaacagctgttacaacatagtagccactctgttgctggtcctcaactttgagaggacaagatcacaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 18 - DNA, 1293 bp, Artificial Sequence
  Nucleic acid sequence of 137DCR4 (including signal peptide)
atgggaaacagctgttacaacatagtagccactctgttgctggtcctcaactttgagaggacaagatcacaggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 19 - DNA, 1098 bp, Artificial Sequence
  Nucleic acid sequence of 137DCR1 (without signal peptide)
caggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 20 - DNA, 1101 bp, Artificial Sequence
  Nucleic acid sequence of 137DCR2 (without signal peptide)
caggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 21 - DNA, 1224 bp, Artificial Sequence
  Nucleic acid sequence of 137DCR3 (without signal peptide)
caggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 22 - DNA, 1224 bp, Artificial Sequence
  Nucleic acid sequence of 137DCR4 (without signal peptide)
caggtgcaactacagcagtggggcgcaggactgttgaagccttcggagaccctgtccctcacctgcgctgtctatggtgggtccttcagtggttactactggagctggatacgccagtccccagagaaggggctggagtggattggggaaatcaatcatggtggatacgtcacctacaatccgtccctcgagagtcgagtcaccatatcagtagacacgtccaagaaccagttctccctgaagctgagctctgtgaccgccgcggacacggctgtatattactgtgcgagggactatggtccggggaattatgactggtacttcgatctctggggccgtggcaccctggtcactgtctcctcaggtggaggcggttcaggcggaggtggcagcggcggtggcgggtcggaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggcgctcactttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 23 - protein, 455 aa, Artificial Sequence
  Amino acid sequence of meso G1 CAR
MALPVTALLLPLALLLHAARPSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIKFVPVFLPAKPTTTPAPRPPTPAPTIASQPLSLRPEACRPAAGGAVHTRGLDFACDIYIWAPLAGTCGVLLLSLVITLYCNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 1371 bp, Artificial Sequence
  Nucleic acid sequence of meso G1 CAR
atggccttaccagtgaccgccttgctcctgccgctggccttgctgctccacgccgccaggccgagccaggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggaggtggcagcggcggtggcgggtcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 25 - protein, 112 aa, Artificial Sequence
  Tyrosine activation motif of CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 2043 bp, Artificial Sequence
  Nucleotide sequence of meso G2 CAR
atggccttaccagtgaccgccttgctcctgccgctggccttgctgctccacgccgccaggccgagcgaggtgcagctggtggagtccgggggaggcctggtccagcctgggggatccctgagactctcctgcgcagcctctggattcgacctcggtttctacttttacgcctgttgggtccgccaggctccagggaagggcctggagtgggtctcatgcatttatactgctggtagtggtagcacgtactacgcgagctgggcgaaaggccgattcaccatctccagagacaattcgaagaacacgctgtatctgcaaatgaacagtctgagagccgaggacacggccgtgtattactgtgcgagatctactgctaatactagaagtacttattatcttaacttgtggggccaaggcaccctggtcaccgtctcctcaggcggaggcggatcaggtggtggcggatctggaggtggcggaagcgacatccagatgacccagtctccatcctccctgtctgcatctgtgggagacagagtcaccatcacttgccaggccagtcagaggattagtagttacttatcctggtatcagcagaaaccagggaaagttcccaagctcctgatctatggtgcatccactctggcatctggggtcccctcgcggttcagtggcagtggatctgggacagatttcactctcaccatcagcagcctgcagcctgaagatgttgccacttactactgtcagagttatgcttattttgatagtaataattggcatgctttcggcggagggaccaaggtggagatcaaagagtccaaatatggtcccccatgcccaccatgcccagcacctcccgtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - DNA, 2718 bp, Artificial Sequence
  Nucleic acid sequence of PS328b vector
ggcgcgccttaaccctagaaagataatcatattgtgacgtacgttaaagataatcatgcgtaaaattgacgcatgtctagaggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcggatcctgcactagtgctgtcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcatgcgtcaattttacgcagactatctttctagggttaaatcgatcccgctagccccgatatccccttaattaagagggggagaccaaagggcgagacgttaaggcctcacgtgacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctctctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccatgcatg

SEQ ID NO: 28 - DNA, 18 bp, Artificial Sequence
  Nucleotide sequence of primer CD137-F
cgagtcaccatatcagta

SEQ ID NO: 29 - DNA, 20 bp, Artificial Sequence
  Nucleotide sequence of primer CD137-R
cgaagtaccagtcataattc

SEQ ID NO: 30 - DNA, 24 bp, Artificial Sequence
  Nucleotide sequence in Taqman
cctcgcacagtaatatacagccgt

SEQ ID NO: 31 - protein, 243 aa, Artificial Sequence
  Amino acid sequence of CD28 extracellular agonistic single-chain antibody
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKE

SEQ ID NO: 32 - protein, 384 aa, Artificial Sequence
  Amino acid sequence of 28DCR1 (including signal peptide)
MGNSCYNIVATLLLVLNFERTRSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 385 aa, Artificial Sequence
  Amino acid sequence of 28DCR2 (including signal peptide)
MGNSCYNIVATLLLVLNFERTRSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 34 - protein, 426 aa, Artificial Sequence
  Amino acid sequence of 28DCR3 (including signal peptide)
MGNSCYNIVATLLLVLNFERTRSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 35 - protein, 426 aa, Artificial Sequence
  Amino acid sequence of 28DCR4 (including signal peptide)
MGNSCYNIVATLLLVLNFERTRSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 36 - protein, 361 aa, Artificial Sequence
  Amino acid sequence of 28DCR1 (without signal peptide)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 37 - protein, 362 aa, Artificial Sequence
  Amino acid sequence of 28DCR2 (without signal peptide)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 38 - protein, 403 aa, Artificial Sequence
  Amino acid sequence of 28DCR3 (without signal peptide)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 39 - protein, 403 aa, Artificial Sequence
  Amino acid sequence of 28DCR4 (without signal peptide)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKESKYGPPCPPCPIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 40 - DNA, 1158 bp, Artificial Sequence
  Nucleic acid sequence of 28DCR1 (including signal peptide)
atgggaaacagctgttacaacatagtagccactctgttgctggtcctcaactttgagaggacaagatcacaggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 41 - DNA, 1161 bp, Artificial Sequence
  Nucleic acid sequence of 28DCR2 (including signal peptide)
atgggaaacagctgttacaacatagtagccactctgttgctggtcctcaactttgagaggacaagatcacaggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 42 - DNA, 1284 bp, Artificial Sequence
  Nucleic acid sequence of 28DCR3 (including signal peptide)
atgggaaacagctgttacaacatagtagccactctgttgctggtcctcaactttgagaggacaagatcacaggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 43 - DNA, 1284 bp, Artificial Sequence
  Nucleic acid sequence of 28DCR4 (including signal peptide)
atgggaaacagctgttacaacatagtagccactctgttgctggtcctcaactttgagaggacaagatcacaggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 44 - DNA, 1089 bp, Artificial Sequence
  Nucleic acid sequence of 28DCR1 (without signal peptide)
caggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 45 - DNA, 1092 bp, Artificial Sequence
  Nucleic acid sequence of 28DCR2 (without signal peptide)
caggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 46 - DNA, 1215 bp, Artificial Sequence
  Nucleic acid sequence of 28DCR3 (without signal peptide)
caggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactgtgataa

SEQ ID NO: 47 - DNA, 1215 bp, Artificial Sequence
  Nucleic acid sequence of 28DCR4 (without signal peptide)
caggtgcagctggtgcagtctggggctgaggtgaagaagcctggggcctcagtgaaggtctcctgcaaggcttctggatacaccttcaccagctactatatacactgggtgcgacaggcccctggacaagggcttgagtggattggatgtatttatcctggaaatgtcaatactaactataatgagaagttcaaggacagggccaccctgaccgtagacacgtccatcagcacagcctacatggagctgagcaggctgagatctgacgacacggccgtgtatttctgtacaagatcacactacggcctcgactggaacttcgatgtctggggccaagggaccacggtcaccgtctcctcaggtggaggcggttcaggcggaggtggcagcggcggtggcgggtcggacatccagatgacccagtctccatcctccctgtctgcatctgtaggagacagagtcaccatcacttgccatgccagtcaaaacatttatgtttggttaaactggtatcagcagaaaccagggaaagcccctaagctcctgatctataaggcttccaacctgcacacaggggtcccatcaaggttcagtggcagtggatctgggacagatttcactctcaccatcagcagtctgcaacctgaagattttgcaacttactactgtcaacagggtcaaacttatccgtacacgttcggcggagggaccaaggtggagatcaaagagtccaaatatggtcccccatgcccaccatgcccaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcctgataa

SEQ ID NO: 48 - protein, 729 aa, Artificial Sequence
  Amino acid sequence of Muc1 G1 CAR
MALPVTALLLPLALLLHAARPSEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGSGSGGSGSGGSGSDIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFVPVFLPAKPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 1371 bp, Artificial Sequence
  Nucleic acid sequence of Muc1 G1 CAR
ccatgcccaccatgcccagcacctcccgtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 50 - DNA, 2187 bp, Artificial Sequence
  Nucleotide sequence of Muc1 G2 CAR
atggccttaccagtgaccgccttgctcctgccgctggccttgctgctccacgccgccaggccgagcgaggtccagctgcagcagtcaggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggttctggttctggcggctccggttccggtggatccggctctgatatcgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatccgaggagtccaaatatggtcccccatgcccaccatgcccagcacctcccgtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 51 - DNA, 18 bp, Artificial Sequence
  Nucleotide sequence of primer CD28-F
gcttctggatacaccttc

SEQ ID NO: 52 - DNA, 24 bp, Artificial Sequence
  Nucleotide sequence of primer CD28-R
ccttgaacttctcattatagttag

SEQ ID NO: 53 - DNA, 23 bp, Artificial Sequence
  Nucleotide sequence in Taqman
aatacatccaatccactcaagcc

SEQ ID NO: 54 - protein, 22 aa, Artificial Sequence
  Amino acid sequence of CD8 signal peptide
MALPVTALLLPLALLLHAARPS

SEQ ID NO: 55 - protein, 248 aa, Artificial Sequence
  Amino acid sequence of CD40 extracellular agonistic single-chain antibody
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 56 - protein, 229 aa, Artificial Sequence
  Amino acid sequence of IgG4Fc CH2CH3 hinge region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 568 aa, Artificial Sequence
  Amino acid sequence of 40DCR1 (including signal peptide)
MALPVTALLLPLALLLHAARPSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVRSKRSRLLHSDYMNMTPRRPGPTRKHYQPYAPPRDFAAYRS

SEQ ID NO: 58 - protein, 569 aa, Artificial Sequence
  Amino acid sequence of 40DCR2 (including signal peptide)
MALPVTALLLPLALLLHAARPSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVKRGRKKLLYIFKQPFMRPVQTTQEEDGCSCRFPEEEEGGCEL

SEQ ID NO: 59 - protein, 610 aa, Artificial Sequence
  Amino acid sequence of 40DCR3 (including signal peptide)
MALPVTALLLPLALLLHAARPSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 60 - protein, 610 aa, Artificial Sequence
  Amino acid sequence of 40DCR4 (including signal peptide)
MALPVTALLLPLALLLHAARPSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 61 - protein, 546 aa, Artificial Sequence
  Amino acid sequence of 40DCR1 (without signal peptide)
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVRSKRSRLLHSDYMNMTPRRPGPTRKHYQPYAPPRDFAAYRS

SEQ ID NO: 62 - protein, 547 aa, Artificial Sequence
  Amino acid sequence of 40DCR2 (without signal peptide)
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVKRGRKKLLYIFKQPFMRPVQTTQEEDGCSCRFPEEEEGGCEL

SEQ ID NO: 63 - protein, 588 aa, Artificial Sequence
  Amino acid sequence of 40DCR3 (without signal peptide)
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 64 - protein, 588 aa, Artificial Sequence
  Amino acid sequence of 40DCR4 (without signal peptide)
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFWVLVVVGGVLACYSLLVTVAFIIFWVKRGRKKLLYIFKQPFMRPVQTTQEEDGCSCRFPEEEEGGCELRSKRSRLLHSDYMNMTPRRPGPTRKHYQPYAPPRDFAAYRS

SEQ ID NO: 65 - DNA, 1704 bp, Artificial Sequence
  Nucleic acid sequence of 40DCR1 (including signal peptide)
atggccttaccagtgaccgccttgctcctgccgctggccttgctgctccacgccgccaggccgagccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 66 - DNA, 1704 bp, Artificial Sequence; identical to SEQ ID NO: 65
  Nucleic acid sequence of 40DCR2 (including signal peptide)

SEQ ID NO: 67 - DNA, 1830 bp, Artificial Sequence
  Nucleic acid sequence of 40DCR3 (including signal peptide)
atggccttaccagtgaccgccttgctcctgccgctggccttgctgctccacgccgccaggccgagccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 68 - DNA, 1830 bp, Artificial Sequence
  Nucleic acid sequence of 40DCR4 (including signal peptide)
atggccttaccagtgaccgccttgctcctgccgctggccttgctgctccacgccgccaggccgagccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 69 - DNA, 1638 bp, Artificial Sequence
  Nucleic acid sequence of 40DCR1 (without signal peptide)
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 70 - DNA, 1638 bp, Artificial Sequence; identical to SEQ ID NO: 69
  Nucleic acid sequence of 28DCR2 (without signal peptide)

SEQ ID NO: 71 - DNA, 1764 bp, Artificial Sequence
  Nucleic acid sequence of 40DCR3 (without signal peptide)
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 72 - DNA, 1764 bp, Artificial Sequence
  Nucleic acid sequence of 40DCR4 (without signal peptide)
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcaggtggaggcggttcaggcggaggtggcagcggcggtggcgggtcggacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaacccttttgggtgctggtggtggttggtggagtcctggcttgctatagcttgctagtaacagtggcctttattattttctgggtgaaacggggcagaaagaagctcctgtatatattcaaacaaccatttatgagaccagtacaaactactcaagaggaagatggctgtagctgccgatttccagaagaagaagaaggaggatgtgaactgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 73 - protein, 499 aa, Artificial Sequence
  Amino acid sequence of EGFR G1 CAR
ATMALPVTALLLPLALLLHAARPSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSFVPVFLPAKPTTTPAPRPPTPAPTIASQPLSLRPEACRPAAGGAVHTRGLDFACDIYIWAPLAGTCGVLLLSLVITLYCNHRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1503 bp, Artificial Sequence
  Nucleic acid sequence of EGFR G1 CAR
gccaccatggccttaccagtgaccgccttgctcctgccgctggccttgctgctccacgccgccaggccgagcgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagctgaaaggtggaggcggttcaggcggaggtggcagcggcggtggcgggtcgcaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctcttcgttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 75 - DNA, 1503 bp, Artificial Sequence; identical to SEQ ID NO: 74
  Nucleotide sequence of EGFR G2 CAR

SEQ ID NO: 76 - DNA, 18 bp, Artificial Sequence
  Nucleotide sequence of primer CD40-F
acctcctgatctatactg

SEQ ID NO: 77 - DNA, 18 bp, Artificial Sequence
  Nucleotide sequence of primer CD40-R
gatggtgagagtgaaatc

SEQ ID NO: 78 - DNA, 21 bp, Artificial Sequence
  Nucleotide sequence in Taqman
cactgccgctgaaccttgatg